(19) United States
(12) Patent Application Publication
(10) Pub. No.: US 2026/0125491 A1
(43) Pub. Date: May 7, 2026

(54) COMPOSITIONS OF LANCL1 ANTIBODIES AND METHODS OF USE THEREOF FOR TREATING LIVER CANCER

(71) Applicant: The University of Hong Kong, Hong Kong (CN)

(72) Inventors: Irene Oi Lin Lui Ng, Hong Kong (CN); Yu Man Tsui, Hong Kong (CN); Chui Yee Diana O, Hong Kong (CN)

(21) Appl. No.: 19/333,895

(22) Filed: Sep. 19, 2025

Related U.S. Application Data
(60) Provisional application No. 63/696,454, filed on Sep. 19, 2024.

(51) Int. Cl. C07K 16/40; A61K 39/00; A61P 35/00

(52) U.S. Cl. CPC C07K 16/40; A61P 35/00; A61K 2039/505; C07K 2317/24; C07K 2317/64; C07K 2317/92

(57) ABSTRACT

Compositions containing anti-LANCL1 antibodies, functional variants thereof, and methods of use thereof, are disclosed. An exemplary anti-LANCL1 antibody has a heavy chain variable region containing the CDRs, SEQ ID NO:5, SEQ ID NO:6, SEQ ID NO:8, respectively, and a light chain variable region containing the CDRs, SEQ ID NO:21, SEQ ID NO:23, SEQ ID NO:24. Another exemplary anti-LANCL1 antibody has a heavy chain variable region containing the CDRs, SEQ ID NO:5, SEQ ID NO:7, SEQ ID NO:9 respectively, and a light chain variable region containing the CDRs, SEQ ID NO:22, SEQ ID NO:73, SEQ ID NO:25, respectively. The antibody can be a monoclonal antibody, a human antibody, a chimeric antibody, or a humanized antibody. The disclosed compositions can be used to treat a subject in need thereof, for example a subject diagnosed with liver cancer or a subject at risk of developing liver cancer e.g., HCC.

### CROSS-REFERENCE TO RELATED APPLICATIONS

This application claims benefit of U.S. Provisional Application No. 63/696,454, filed Sep. 19, 2024, is hereby incorporated herein by reference in its entirety.

### REFERENCE TO THE SEQUENCE LISTING

The Sequence Listing submitted as an XML file named “UHK_01547_US_ST26”, created Dec. 8, 2025, and having a size of 81,030 bytes is hereby incorporated by reference pursuant to 37 C.F.R. § 1.834 (c)(1).

### FIELD OF THE INVENTION

The disclosed invention is generally in the field of liver cancer and specifically in the area of therapeutic antibodies for treating liver cancer.

### BACKGROUND OF THE INVENTION

Hepatocellular Carcinoma (HCC) is the third leading cause of cancer deaths globally, and more specifically, the second and third leading cause of cancer deaths in China and Hong Kong, respectively. Although surgical resection and liver transplantation are available, most HCCs are diagnosed at advanced stages and thus inoperable. The median survival rate of patients with inoperable HCC is in the order of weeks. Even if operated on, the tumor recurrence rate is high. The 5-year survival rate of HCC patients, even after surgical removal of the cancer, is only ˜20%. The high mortality rate of HCC is attributed, in part, to its aggressive behavior and lack of promising curative therapy. HCC is notoriously resistant to conventional chemotherapy, hence treatments for advanced HCCs are limited.

Tumor initiation is crucial in both hepatocarcinogenesis, and tumor recurrence derived from residual tumor cell population after treatment. Targeting the cell surface proteins responsible for tumor initiation is one potential strategy for cancer treatment. Existing cell surface proteins that promote liver tumor initiation include CD13, CD24, CD47, CD90, CD133, and EpCAM. However, these proteins are limited for translational applications. For example, some of these proteins have low expression in terms of either a very low percentage of positive cells in a tumor or very few patients' samples showing detectable expression (such as CD90), implicating a lesser role of the protein in HCC initiation in a certain cohort of patients. Also, some surface proteins are detectable not only in HCC tumors but also at high levels in non-tumorous liver tissues. Targeting these proteins may cause a nonspecific cytotoxic effect on the surrounding non-tumorous liver tissues. Thus, there is an urgent need for more effective therapies for precise targeting of cell surface proteins for the treatment of HCC.

It is an object of the present invention to provide monoclonal antibodies that specifically bind to LanC Like Glutathione S-Transferase 1 (LANCL1) protein or a portion of human LANCL1 protein.

It is also an object of the present invention to provide compositions for treating HCC in a subject in need thereof.

It is also an object of the present invention to provide methods for treating HCC in a subject.

It is still an object of the present invention to provide methods for inhibiting HCC tumor growth in a subject in need thereof.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### SUMMARY OF THE INVENTION

Liver tumor-initiating cells (LTICs) are self-renewing sub-populations of cells in Hepatocellular Carcinoma (HCC) which contribute to initiation of tumors and can be considered the culprit for resistance to HCC treatment (Yi, et al., Cancer Treat Rev., 39:290-296 (2013); Visvader and Lindeman, Nat Rev Cancer, 8:755-768 (2008); Tirino et al., FASEB J., 27:13-24 (2013)). LTICs are marked by unique cell-surface markers which are functional and can activate downstream cell signaling to exert their effects. The problem is that existing LTIC markers are limited in their capability for translational applications, thereby hindering the development of therapeutic applications against LTICs for cancer therapy. The disclosed compositions and methods solve this problem by providing human monoclonal antibodies which reduce the activity of LTIC surface markers and decrease the downstream signaling pathways triggered by these surface markers, thereby decreasing initiation of hepatocellular carcinoma.

It was realized that development of human monoclonal antibodies which target and inhibit the activity of LanC Like Glutathione S-Transferase 1 (LANCL1), a LTIC marker common in hepatocellular carcinoma patients, was useful for reducing the proliferation of HCC cells, reducing the development of HCC, and/or increasing the survival rate of liver cancer patients. To date, all available antibodies against LANCL1 can only be used for Western Blot, immunohistochemistry or immunofluorescence applications. None of the existing LANCL1 antibodies available have cancer therapeutic capabilities.

Disclosed are antibodies or antigen-binding fragments thereof, that immunospecifically bind to a surface unit or a transmembrane unit of human LanC Like Glutathione S-Transferase 1 (LANCL1) protein and are capable of blocking or reducing the activity of LANCL1 in vitro, or in a recipient subject or patient. The antibodies and other molecules typically include six complementarity determining regions (CDRs). In preferred forms the antibodies and other molecules immunospecifically bind to the N-terminal extracellular region of the amino acid sequence SEQ ID NO:1. In some forms, the N-terminal extracellular region of LANCL1 has the amino acid sequence SEQ ID NO:2.

The anti-LANCL1 antibodies contain a heavy chain variable region having three complementarity determining regions (CDRs) and a light chain variable region having three CDRs. In one particular form, the three heavy chain variable region CDRs are DYYMN (SEQ ID NO:5), DINPNNGGASYNQKFKG (SEQ ID NO:6), SGDGYYFAS (SEQ ID NO:8), respectively, and the three light chain variable region CDRs contain RASKSVSTSGYSYMH (SEQ ID NO:21), LVSNLES (SEQ ID NO:23), QHIRELT (SEQ ID NO:24) respectively. In another particular form, the three heavy chain variable region CDRs include DYYMN (SEQ ID NO:5), VINPYNGHTNYNQKFKG (SEQ ID NO:7), FPYYGSSYRVDY (SEQ ID NO:9) respectively, and the three light chain variable region CDRs include RASQSISNNLH (SEQ ID NO:22), YASQSIS (SEQ ID NO:73), QQINSWPLT (SEQ ID NO:25), respectively.

The disclosed subject matter can also involve chimeric and/or humanized antibodies and fragments, or human antibodies and fragments. Most preferably, such molecules will possess sufficient affinity and avidity to be able to bind to extracellular LANCL1 when present in a subject.

Also provided are nucleic acids encoding the antibody or antigen-binding fragments.

Pharmaceutical compositions containing the anti-LANCL1 antibodies are also disclosed.

Methods for treating a subject in need thereof, for example, a subject diagnosed with liver cancer e.g., hepatocellular carcinoma (HCC), or a subject at risk of developing liver cancer e.g., HCC are also disclosed.

The methods generally include administering to the subject a pharmaceutical composition containing an effective amount of the anti-LANCL1 antibodies to the subject. In some forms, the pharmaceutical composition is effective to reduce the proliferation of tumor-initiating cells, sphere formation of hepatocellular carcinoma cells, and/or blocking or reducing the activity of LANCL1.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIGS. 1A to 1F show the generation and screening of LANCL1 monoclonal antibodies. FIG. 1A is a table of ELISA results for hybridoma clone supernatant. FIG. 1B are western blots using the respective hybridoma clone supernatants at 1:1000 dilution to probe for the denatured GST-tagged human LANCL1 1-42aa peptide overexpressed in E. coli BL21 lysate. The ladder scales are in kDa. FIG. 1C are western blots using the respective hybridoma clone supernatants at 1:1000 dilution to probe for the non-denatured GST-tagged human LANCL1 1-42aa peptide expressed in E. coli BL21 lysate. The ladder scales are in kDa. FIG. 1D is a table summary for the detectability by respective hybridoma clone supernatants for both the denatured and non-denatured GST-tagged human LANCL1 1-42aa peptides overexpressed in the E. coli BL21 lysate. FIG. 1E are western blots using the respective hybridoma clone supernatants at 1:250 dilution to probe for the non-denatured His-tagged full-length human LANCL1 protein overexpressed in PLC/PRF/5 lysate. The ladder scales are in kDa. FIG. 1F are western blots using the respective hybridoma clone supernatants at 1:100 dilution to probe for both the denatured and non-denatured His-tagged full-length human LANCL1 protein overexpressed in PLC/PRF/5 lysate. The ladder scales are in kDa.

FIG. 2 shows that LANCL1 monoclonal antibodies 4D9 and 19G4 suppressed sphere formation of PLC/PRF/5 HCC cell line. 700 PLC/PRF/5 cells were seeded in 96-well with 100 μl content. Three independent trials performed (unpaired t test).

FIGS. 3A to 3C shows testing the effects of LANCL1 monoclonal antibodies 4D9 and 19G4 on the growth of PLC/PRF/5 HCC xenograft in vivo. 5e5 PLC/PRF/5 HCC cells in 0.5× growth factor-reduced Matrigel were subcutaneously injected per site on a single flank of each NOD-SCID mouse. When palpable tumors were observed on day 9 after the PLC/PRF/5 cell injection, the first doses of the monoclonal antibodies and the corresponding IgG ctrl were injected intraperitoneally at 12 mg/kg. Two doses were administered per week for two weeks as indicated. FIG. 3A shows that the mouse weights were not affected by the administration of the 4D9 and 19G4 monoclonal antibodies as compared to the IgG control. FIG. 3B. PLC/PRF/5 xenograft growth was significantly suppressed by 19G4 but not 4D9 monoclonal antibodies as compared to the IgG control at 12 mg/kg dose as observed on Day 19 after the PLC/PRF/5 cell injection (unpaired t test). FIG. 3C shows that tumors collected at end-point (Day 21 after PLC/PRF/5 cell injection) showed reduced tumor sizes for 4D9 and 19G4 monoclonal antibody treatment as compared to the IgG control.

FIG. 4A-4C. Testing the effects of LANCL1 monoclonal antibody 19G4 on the mouse orthotopic HCC model. 1×10⁶ luciferase-labelled PLC/PRF/5 HCC cells in 1× Matrigel were orthotopically injected into the left lobe of the liver of each NOD-SCID mouse. When tumors were observed to be formed by luciferase signal imaging 2 weeks after the orthotopic injection, the mice were randomized into respective groups to receive the 19G4 monoclonal antibody, the corresponding mouse IgG control or no treatment. The antibodies were injected intraperitoneally at 60 mg/kg. Two doses were administered per week for two weeks. (FIG. 4A, 4B) Liver tumor size as indicated by the luciferase signal imaging of the mice and the extracted livers respectively at end point. (FIG. 4C) Lung metastasis of the PLC/PRF/5 HCC cells was significantly suppressed by 19G4 monoclonal antibody as compared to the IgG ctrl as determined by the luciferase signaling imaging of the extracted lung tissues (unpaired t test).

FIG. 5A-5B. The cytotoxic activity of 19G4 monoclonal antibody on liver cancer cells was evaluated by (FIG. 5A) cytotoxicity assays, which help determine the IC50, and (FIG. 5B) Annexin V assays by flow cytometry. (FIG. 5A) In the cytotoxicity assays, 2000 PLC/PRF/5 cells seeded per well on a 96-well plate were treated with the eight different indicated concentrations of the 19G4 monoclonal antibody or the IgG control respectively. After 24 hours, cell viability was assessed using DAPI staining and subsequent cell counting. The IC50 value of 19G4 monoclonal antibody was determined to be 35.29 μg/ml. (FIG. 5B) The cytotoxic activity of the 19G4 monoclonal antibody was further validated by Annexin V/PI staining. In these assays, cells were seeded at 10,000 cells per well on a 24-well plate with four wells for each condition, and immediately after cell seeding, the wells of cells were treated with 128 μg/ml of 19G4 monoclonal antibody or the IgG control respectively. A total of 1×10⁵ PLC/PRF/5 cells were harvested for each condition for flow cytometry, with 10,000 events analyzed per sample. A 48-hour treatment of the 19G4 monoclonal antibody showed trend of increase in apoptosis but not yet reaching statistical significance as compared to the IgG control. Flow cytometry diagram for one representative trial is shown in the left panel, while shown in the right panel is a summary of three independent trials (paired t test).

FIG. 6A-6B. Effects of the 19G4 monoclonal antibody on the migratory and invasive abilities of the HCC cells were assessed by transwell migration and invasion 20) assays respectively. (FIG. 6A) In the transwell migration assays, 1×10⁵ PLC/PRF/5 cells were seeded in the upper chamber, with 128 μg/mL of the 19G4 monoclonal antibody applied to both the upper and lower chambers. The lower chamber contained 10% FBS as a chemoattractant whereas the upper chamber had serum-free medium. After 6 hours of incubation at 37° C., the number of cells that had migrated across the membrane to the bottom surface of the membrane in the transwell were stained and counted in random views. 19G4 antibody significantly reduced HCC cell migration as compared to the IgG control. (FIG. 6B) In the transwell invasion assays, the upper chamber was pre-coated with 3 mg/ml Matrigel before seeding of 5×10⁴ PLC/PRF/5 cells. All other conditions were the same as the migration assays, except for an extended incubation period of 72 hours. The 19G4 antibody significantly decreased HCC cell invasion as compared to the IgG control. Three independent trials were performed for all assays and statistical significance was analyzed by paired t test.

FIG. 7A-7B. By using the 19G4 monoclonal antibody, flow cytometry analyses were performed to determine (FIG. 7A) the mean fluorescence intensity (MFI) of the total cell population and (FIG. 7B) the percentages of the cells positive for the expression of LANCL1 in the unpermeabilized PLC/PRF/5 cells collected at different numbers of days of spheroid and routine adherent culture. The expression of LANCL1 increased in spheroid culture but not in adherent culture, implicating the enrichment of LANCL1-positive cells in spheroid culture.

FIG. 8A-8C. Schematic diagrams showing the humanization of the 19G4 monoclonal antibodies. (FIG. 8A) The methodology for the design of the humanized monoclonal antibodies. (FIG. 8B) The structures of the original non-humanized 19G4 antibody and the humanized chimeric 19G4 monoclonal antibody. (FIG. 8C) The structures of the different variants of the humanized 19G4 monoclonal antibodies. CDR, complementarity determining region. Figures were created in BioRender.

FIG. 9A-9C. Affinity measurement for the humanized 19G4 monoclonal antibody variants by Biacore. (FIG. 9A) Schematic diagram of Biacore measurement. (FIG. 9B) The analyte, ligand and the capturing molecule used in Biacore measurement. (FIG. 9C) Raw Biacore data for the affinity measurement of the various humanized antibody variants. (FIG. 9D-9E) The results for the Biacore affinity measurement for the humanized 19G4 monoclonal antibody variants. Equilibrium dissociation constant (KD) that indicated the overall binding affinity was calculated by the formula, KD=kd/ka; whereas kd, the dissociation rate constant, indicates how fast unbinding occurs and ka, the association rate constant indicates how fast binding occurs. The closer the KD values of the antibody variants to that of the chimeric humanized antibody (VH+VL), the more similar their affinity to the LANCL1 1-42aa region is.

FIG. 10A, Results for the post-translational modification (PTM) risk prediction of the heavy chains of the humanized 19G4 antibody variants. Blue-highlighted amino acid residues are of moderate risk of PTM while the green-highlighted ones have low risk of PTM. FIG. 10B. Results for the post-translational modification (PTM) risk prediction of the light chains of the humanized 19G4 antibody variants. Blue-highlighted amino acid residues are of moderate risk of PTM while the green-highlighted ones have low risk of PTM.

FIG. 11A-11B. Sphere formation assays using PLC/PRF/5 HCC cell lines for testing the various humanized 19G4 monoclonal antibody variants. The number of spheres formed on (FIG. 11A) Day 8 and (FIG. 11B) Day 11 were counted. The humanized antibody variants VH2+VL2 and VH3+VL2 significantly suppressed sphere formation, similar to the chimeric humanized antibody VH+VL. Three independent trials were performed with paired t test done for statistical significance analysis.

### DETAILED DESCRIPTION OF THE INVENTION

The disclosed method and compositions can be understood more readily by reference to the following detailed description of particular embodiments and the Example included therein and to the Figures and their previous and following description.

### I. Definitions

The term “antibody” is used in the broadest sense unless clearly indicated otherwise. Therefore, an “antibody” can be naturally occurring or man-made such as monoclonal antibodies produced by conventional hybridoma technology. Antibodies include monoclonal and polyclonal antibodies as well as fragments and polymers containing the antigen binding domain and/or one or more complementarity determining regions of these antibodies. As used herein, the term “antibody” refers to any form of antibody or antigen binding fragment or recombinant protein, and specifically covers monoclonal antibodies (including full length monoclonal antibodies), polyclonal antibodies, multispecific antibodies (e.g., bispecific antibodies), and antibody fragments so long as they specifically bind the target antigen. Any specific antibody can be used in the methods and compositions provided herein. The term “antibody” encompasses an immunoglobulin molecule that possesses a “variable region” antigen recognition site. Thus, the term “antibody” encompasses a molecule having at least one variable region from a light chain immunoglobulin molecule and at least one variable region from a heavy chain molecule that in combination form a specific binding site for the target antigen. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to the disclosed antibodies). In particular, such antibodies include immunoglobulin molecules of any type (e.g., IgG, IgE, IgM, IgD, IgA and IgY), class (e.g., IgG1, IgG2, IgG3, IgG4, IgA1 and IgA2) or subclass.

As used herein, the term “monoclonal antibody” or “MAb” refers to an antibody obtained from a substantially homogeneous population of antibodies, i.e., the individual antibodies within the population are identical except for possible naturally occurring mutations that may be present in a small subset of the antibody molecules.

The term “variable region” is intended to distinguish such domain of the immunoglobulin from domains that are broadly shared by antibodies (such as an antibody Fc domain). The variable region refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region can include three or four FRs (e.g., FR1, FR2, FR3 and optionally FR4) together with three CDRs. VH refers to the variable region of the heavy chain. VL refers to the variable region of the light chain. The variable region includes a “hypervariable region” whose residues are responsible for antigen binding.

The hypervariable region includes amino acid residues from a “Complementarity Determining Region” or “CDR” (e.g., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain according to Kabat; Kabat et al., Sequences of Proteins of Immunological Interest, 5th Ed. Public Health Service, National Institutes of Health, Bethesda, MD. (1991)) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain according to Chothia; Chothia and Lesk, 1987, J. Mol. Biol. 196:901-917). Conventions that include corrections or alternate numbering systems for variable domains include not only Kabat and Chothia, but also IMGT (Lefranc, et al. (2003), Dev Comp Immunol 27:55-77), Chothia (Chothia C, Lesk A M (1987), J Mal Biol 196:901-917; Chothia, et al. (1989), Nature 342:877-883) and AHo (Honegger A, Plückthun A (2001) J Mol Biol 309:657-670). For convenience, examples of binding proteins of the present disclosure may also be labelled according to Kabat, Chothia, or IMGT. These examples are expressly indicated as such.

“Framework Region” or “FR” residues are those variable domain residues other than the hypervariable region residues as herein defined.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab, Fab′, F(ab′)2 and Fv fragments; diabodies; linear antibodies; single-chain antibody molecules and multispecific antibodies formed from antibody fragments, and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.). For example, the term antigen binding fragment may be used to refer to recombinant single chain Fv fragments (scFv) as well as divalent (di-scFv) and trivalent (tri-scFV) forms thereof. Such fragments can be produced via various methods known in art.

As used herein, the term “single chain Fv” or “scFv” as used herein means a single chain variable fragment that includes a light chain variable region (VL) and a heavy chain variable region (VH) in a single polypeptide chain joined by a linker which enables the scFv to form the desired structure for antigen binding (i.e., for the VH and VL of the single polypeptide chain to associate with one another to form a Fv). The VL and VH regions may be derived from the parent antibody or may be chemically or recombinantly synthesized.

The term “constant region” as used herein, refers to a portion of heavy chain or light chain of an antibody other than the variable region. In a heavy chain, the constant region generally includes a plurality of constant domains and a hinge region, e.g., an IgG constant region includes the following linked components, a constant heavy C_{H}1, a linker, a C_{H}2 and a C_{H}3. In a heavy chain, a constant region includes a Fc. In a light chain, a constant region generally include one constant domain (a CL1).

The term “fragment crystalizable” or “Fc” or “Fc region” or “Fc portion” (which can be used interchangeably herein) refers to a region of an antibody including at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Exemplary heavy chain constant regions are gamma 1 (IgG1), gamma 2 (IgG2) and gamma 3 (IgG3), or hybrids thereof.

A “constant domain” is a domain in an antibody the sequence of which is highly similar in antibodies/antibodies of the same type, e.g., IgG or IgM or IgE. A constant region of an antibody generally includes a plurality of constant domains, e.g., the constant region of γ, α or δ heavy chain include two constant domains.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

A “chimeric antibody” is a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 6,311,415, 5,807,715, 4,816,567, and 4,816,397. Chimeric antibodies including one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, Proc. Natl. Acad. Sci. USA 91:969), and chain shuffling (U.S. Pat. No. 5,565,332).

As used herein, the term “humanized antibody” refers to an immunoglobulin including a human framework region and one or more CDR's from a non-human (usually a mouse or rat) immunoglobulin. The non-human immunoglobulin providing the CDR's is called the “donor” and the human immunoglobulin providing the framework is called the “acceptor.”

As used herein, the term “fragment” refers to a peptide or polypeptide including an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred. Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

As used herein, the term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

As used herein, the term “binds” in reference to the interaction of a binding protein and an antigen means that the interaction is dependent upon the presence of a particular structure (e.g., an antigenic determinant or epitope) on the antigen. For example, a binding protein recognizes and binds to a specific antigen structure rather than to antigens generally. For example, if a binding protein binds to epitope “A”, the presence of a molecule containing epitope “A” (or free, unlabeled “A”), in a reaction containing labeled “A” and the binding protein, will reduce the amount of labeled “A” bound to the binding protein.

As used herein, the term “specifically binds” refers to the binding of an antibody to its cognate antigen (for example, guanosine) while not significantly binding to other antigens. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

As used herein, the phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

As used herein, the term “carrier” or “excipient” refers to an organic or inorganic ingredient, natural or synthetic inactive ingredient in a formulation, with which one or more active ingredients are combined. The carrier or excipient would naturally be selected to minimize any degradation of the active ingredient and to minimize any adverse side effects in the subject, as would be well known to one of skill in the art.

As used herein, the term “individual,” “subject,” and “patient” are used interchangeably to refer to any individual who is the target of administration or treatment. The subject can be a vertebrate, for example, a mammal. Thus, the subject can be a human or veterinary patient. The term does not denote a particular age or sex.

As used herein, the term “treat” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term ‘treating HCC’” or “HCC treatment” means reducing, inhibiting, or alleviating one or more symptoms related to HCC in a subject suffering from HCC.

As used herein, the term “inhibit” or “reduce” means to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the term “effective amount” means that the amount of the composition used is of sufficient quantity to ameliorate one or more causes or symptoms of a disease or disorder. Such amelioration only requires a reduction or alteration, not necessarily elimination. The precise dosage will vary according to a variety of factors such as subject-dependent variables (e.g., age, immune system health, etc.), the disease or disorder being treated, as well as the route of administration and the pharmacokinetics of the agent being administered. As used herein, the term “therapeutically effective amount” refers to an amount of the therapeutic agent that, when incorporated into and/or onto particles described herein, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders of the brain, such as reducing tumor size (e.g., tumor volume).

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### II. Compositions

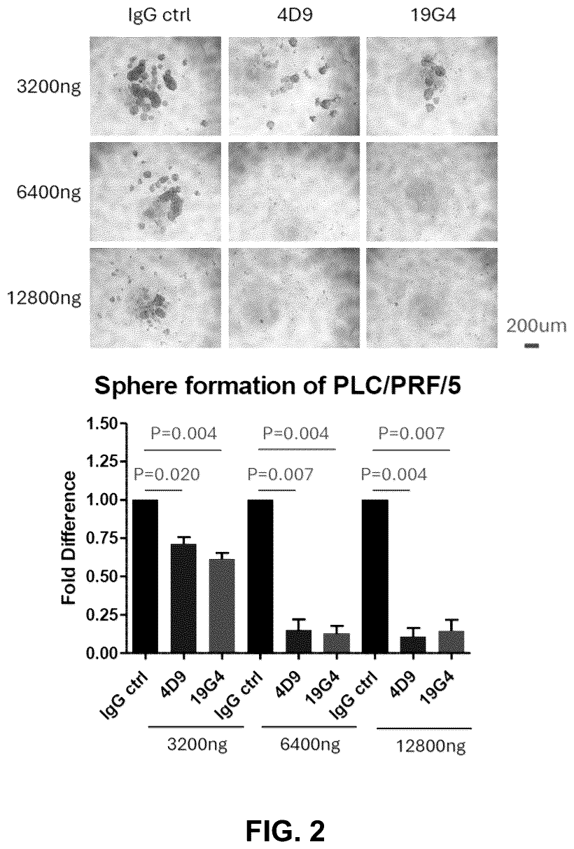

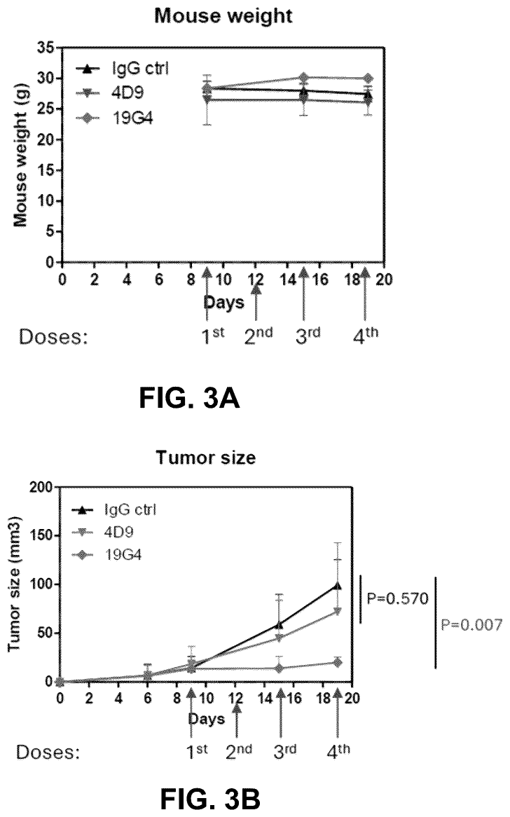

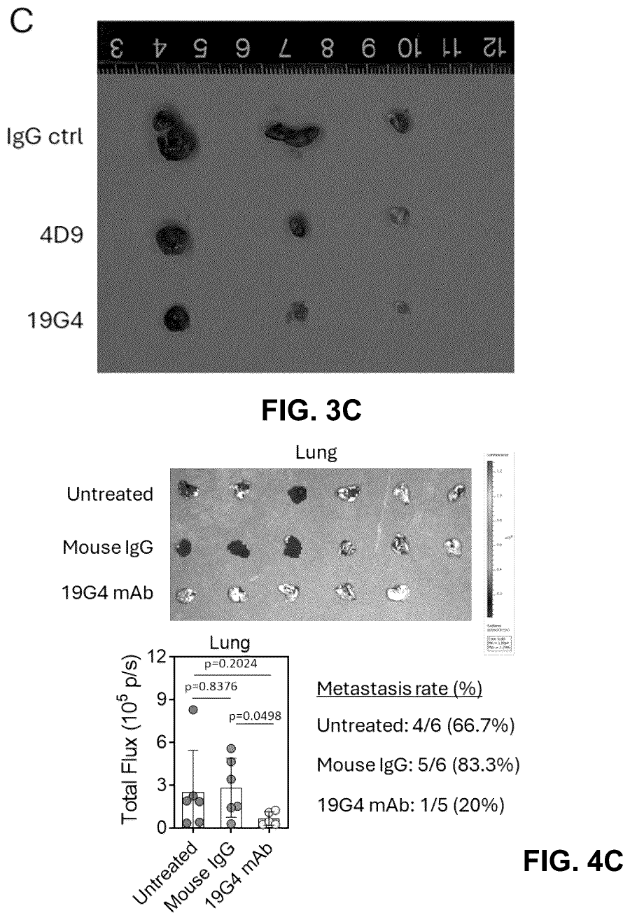

Compositions of anti-LANCL1 monoclonal antibodies and antigen-binding fragments thereof, are provided. The disclosed compositions are based, in part, on the discovery that anti-LANCL1 monoclonal antibodies can suppress the pro-tumorigenic features of hepatocellular carcinoma cells such as sphere formation (FIG. 2), and in vivo tumorigenicity (FIGS. 3B and 3C). Based on this discovery, anti-LANCL1 monoclonal antibodies (mAbs) were created and sequenced, and the heavy chain and light chain variable domains were identified.

An exemplary consensus amino acid sequence for human LANCL1 protein is represented by SEQ ID NO:1.

Human LANCL1 protein (399 aa; UniProt ID: O43813 LANC1_HUMAN)
(SEQ ID NO: 1)
MAQRAFPNPYADYNKSLAEGYFDAAGRLTPEFSQRLINKIRELLQQMERGLKSADPRDG
TGYTGWAGIAVLYLHLYDVFGDPAYLQLAHGYVKQSLNCLTKRSITFLCGDAGPLAVAA
VLYHKMNNEKQAEDCITRLIHLNKIDPHAPNEMLYGRIGYIYALLFVNKNFGVEKIPQS
HIQQICETILTSGENLARKRNFTAKSPLMYEWYQEYYVGAAHGLAGIYYYLMQPSLQVS
QGKLHSLVKPSVDYVCQLKFPSGNYPPCIGDNRDLLVHWCHGAPGVIYMLIQAYKVFRE
EKYLCDAYQCADVIWQYGLLKKGYGLCHGSAGNAYAFLTLYNLTQDMKYLYRACKFAEW
CLEYGEHGCRIPDTPFSLFEGMAGTIYFLADLLVPTKARFPAFEL

The antigen used to prepare the disclosed antibodies and antigen-binding fragments contains the 1-42 aa region specific to the LANCL1 protein: MAQRAFPNPYADYNKSLAEGYFDAAGRLTPEFSQRLTNKIRE (SEQ ID NO:2). In some forms, the antibodies bind to a fragment of human LANCL1 e.g., human LANCL1 having the sequence of SEQ ID NO:2. In some forms, a Cys (C) residue is added to SEQ ID NO:2 to allow KLH conjugation for the purpose of immunizing rodents e.g., mice. Generally, this Cys amino acid residue is not naturally present in the corresponding LANCL1 full-length protein.

In some embodiments, the antibodies bind to a variant having at least 60% up to 99% identity to SEQ ID NO:1 or SEQ ID NO:2. For example in some forms, the antibodies bind to a variant sequence of human LANCL1 having about 70%, about 75%, about 80%, about 85%, about 90%, about 95% or about 99% sequence identity to SEQ ID NO:1 or SEQ ID NO:2. Therefore, in some forms, the variant consensus amino acid sequence for the human LANCL1 protein has an amino acid sequence that has one or more amino acids different to SEQ ID NO:1 or SEQ ID NO:2, such as one or more substitutions, deletions or additions at any one of the amino acid positions of SEQ ID NO:1 or SEQ ID NO:2.

As discussed herein, antibodies that bind human LANCL1 protein, having one or more of the associated CDRs or variant thereof, and/or one or both the associated VH and VL sequences or variant thereof, in any and all antibody forms including, but not limited to, intact antibodies and antigen binding fragments, in monospecific, bispecific, and higher order multispecific formats, optionally as humanized or chimeric forms thereof are expressly provided. Thus, in some embodiments, the antibodies are, or include fragments with antigen-binding capability (e.g., Fab′, F(ab′)₂, Fab, Fv and rIgG, recombinant single chain Fv fragments (scFv) as well as divalent (di-scFv) and trivalent (tri-scFV) forms thereof.

### A. Sequences for Anti-LANCL1 Monoclonal Antibodies

### 1. Heavy Chain Variable Region

In some forms, an amino acid sequence for the kappa heavy chain variable region (VH) of the anti-LANCL1 monoclonal antibody (4D9) has the sequence:

(SEQ ID NO: 3)
MGWSWIFLFLLSGTAGVLSEVQLQQSGPELVKPGASVKISCKASGYTFTDYYMNWVKQS
HGKSLEWIGDINPNNGGASYNQKFKGKATLTVDKSSSTAYMELRSLTSEDSAVYYCVRS
GDGYYFASWGQGTLLTVSAAKTTPPSVYPLAPGCGDTTGSSVTLGCLVKGYFPESVTVT
WNSGSLSSSVHTFPALLQSGLYTMSSSVTVPSSTWPSQTVTCSVAHPASSTTVDKKLEP
SGPISTINPCPPCKECHKCPAPNLEGGPSVFIFPPNIKDVLMISLTPKVTCVVVDVSED
DPDVRISWFVNNVEVHTAQTQTHREDYNSTIRVVSALPIQHQDWMSGKEFKCKVNNKDL
PSPIERTISKIKGLVRAPQVYILPPPAEQLSRKDVSLTCLVVGFNPGDISVEWTSNGHT
EENYKDTAPVLDSDGSYFIYSKLDIKTSKWEKTDSFSCNVRHEGLKNYYLKKTISRSPG
K-.

In some forms, an amino acid sequence for the kappa heavy chain variable region (VH) of the anti-LANCL1 monoclonal (19G4) antibody has the sequence:

(SEQ ID NO: 4)
MGWSWIFLFLLSGTAGVHSEVQLQQSGPVLVKPGASVKMSCKASGYTFTDYYMNWVKQS
HGKSLEWIGVINPYNGHTNYNQKFKGKATLTVDKSSNTAYMELNSLTSEDSAVYYCARF
PYYGSSYRVDYWGQGTTLTVSSAKTTPPSVYPLAPGCGDTTGSSVTLGCLVKGYFPESV
TVTWNSGSLSSSVHTFPALLQSGLYTMSSSVTVPSSTWPSQTVTCSVAHPASSTTVDKK
LEPSGPISTINPCPPCKECHKCPAPNLEGGPSVFIFPPNIKDVLMISLTPKVTCVVVDV
SEDDPDVRISWFVNNVEVHTAQTQTHREDYNSTIRVVSALPIQHQDWMSGKEFKCKVNN
KDLPSPIERTISKIKGLVRAPQVYILPPPAEQLSRKDVSLTCLVVGFNPGDISVEWTSN
GHTEENYKDTAPVLDSDGSYFIYSKLDIKTSKWEKTDSFSCNVRHEGLKNYYLKKTISR
SPGK-.

The complementarity determining regions (CDRs) are shown in bold italic in SEQ ID NO:3 and SEQ ID NO:4, including a first, second, and third CDR regions, CDR-1, CDR-2, and CDR-3 respectively. An exemplary amino acid sequence for CDR-1 of the heavy chain variable region is DYYMN (SEQ ID NO:5). Exemplary amino acid sequences for CDR-2 of the heavy chain variable region include DINPNNGGASYNQKFKG (SEQ ID NO:6), and VINPYNGHTNYNQKFKG (SEQ ID NO:7). Exemplary amino acid sequences for CDR-3 of the heavy chain variable region include SGDGYYFAS (SEQ ID NO:8) and FPYYGSSYRVDY (SEQ ID NO:9). In some forms, the first, second, and third CDR regions of the heavy chain variable region include variant amino acid sequences having about 70%, about 80%, about 90%, about 95%, about 96%, about 97%, about 98%, about 99% sequence identity to SEQ ID NO:5, SEQ ID NO:6, SEQ ID NO:7, SEQ ID NO:8, and SEQ ID NO:9 respectively.

The framework region (FR) regions are shown in underlining in SEQ ID NO:3 and SEQ ID NO:4, including a first, second, third, and fourth FR regions, FR1, FR2, FR3, and FR4 respectively. Exemplary amino acid sequences for FR1 of the heavy chain variable region include EVQLQQSGPELVKPGASVKISCKASGYTFT (SEQ ID NO:10) and EVQLQQSGPVLVKPGASVKMSCKASGYTFT (SEQ ID NO:11). An exemplary sequence for FR2 of the heavy chain variable region is WVKQSHGKSLEWIG (SEQ ID NO:12). Exemplary sequences for FR3 of the heavy chain variable region include KATLTVDKSSSTAYMELRSLTSEDSAVYYCVR (SEQ ID NO:13) and KATLTVDKSSNTAYMELNSLTSEDSAVYYCAR (SEQ ID NO:14). Exemplary sequences for FR4 of the heavy chain variable region include WGQGTLLTVSA (SEQ ID NO:15) and WGQGTTLTVSS (SEQ ID NO:16). In some forms, the first, second, third, and fourth FR regions of the heavy chain variable region include variant amino acid sequences having about 70%, about 80%, about 90%, about 95%, about 96%, about 97%, about 98%, about 99% sequence identity to SEQ ID NO:10, SEQ ID NO:11, SEQ ID NO:12, SEQ ID NO:13, SEQ ID NO:14, SEQ ID NO:15, and/or SEQ ID NO:16 respectively.

The signal peptides for the heavy chain variable region are shown in italic in SEQ ID NO:3 and SEQ ID NO:4. Exemplary sequences for the signal peptide of the heavy chain variable region include MGWSWIFLFLLSGTAGVLS (SEQ ID NO:17) and MGWSWIFLFLLSGTAGVHS (SEQ ID NO:18). In some forms, the signal peptide of the heavy chain variable region includes variant amino acid sequences having about 70%, about 80%, about 90%, about 95%, about 96%, about 97%, about 98%, about 99% sequence identity to SEQ ID NO:17 or SEQ ID NO:18.

In some forms, the constant region of the heavy chain variable region can include the amino acid sequence:

(SEQ ID NO: 36)
AKTTPPSVYPLAPGCGDTTGSSVTLGCLVKGYFPESVTVTWNSGSLSSSVHTFPALLQS
GLYTMSSSVTVPSSTWPSQTVTCSVAHPASSTTVDKKLEPSGPISTINPCPPCKECHKC
PAPNLEGGPSVFIFPPNIKDVLMISLTPKVTCVVVDVSEDDPDVRISWFVNNVEVHTAQ
TQTHREDYNSTIRVVSALPIQHQDWMSGKEFKCKVNNKDLPSPIERTISKIKGLVRAPQ
VYILPPPAEQLSRKDVSLTCLVVGFNPGDISVEWTSNGHTEENYKDTAPVLDSDGSYFI
YSKLDIKTSKWEKTDSFSCNVRHEGLKNYYLKKTISRSPGK.

In some forms, the constant region of the heavy chain variable region includes variant amino acid sequences having about 70%, about 80%, about 90%, about 95%, about 96%, about 97%, about 98%, about 99% sequence identity to SEQ ID NO:36.

### 2. Light Chain Variable Region

In some forms, an amino acid sequence for the kappa light chain variable region (VL) of the anti-LANCL1 monoclonal (4D9) antibody has the sequence:

(SEQ ID NO: 19)
MGTAALGSRFHWDIVLTQSPASLAVSLGQRATISYRASKSVSTSGYSYMHWNQQKPGQP
PRLLIYLVSNLESGVPARFSGSGSGTDFTLNIHPVEEEDAATYYCQHIRELTFGGGTKL
EIKRADAAPTVSIFPPSSEQLTSGGASVVCFLNNFYPKDINVKWKIDGSERQNGVLNSW
TDQDSKDSTYSMSSTLILIKDEYERHNSYTCEATHKTSTSPIVKSENRNEC-..

IN some forms, an amino acid sequence for the kappa light chain variable region (VL) of the anti-LANCL1 monoclonal (19G4) antibody has the sequence:

(SEQ ID NO: 20)
MVFTPQILGLMLFWISASRGDIVLTQSPATLSVTPGDSVSLSCRASQSISNNLHWYQQK
SHESPRLLIKYASQSISGIPSRFSGSGSGTDFTLSINSVETEDFGMYFCQQINSWPLTF
GAGTKLELKRADAAPTVSIFPPSSEQLTSGGASVVCFLNNFYPKDINVKWKIDGSERQN
GVLNSWTDQDSKDSTYSMSSTLTLTKDEYERHNSYTCEATHKTSTSPIVKSENRNEC-.

The complementarity determining regions (CDRs) are shown in bold italic in SEQ ID NO:19 and SEQ ID NO:20, including a first, second, and third CDR regions, CDR-1, CDR-2, and CDR-3 respectively. Exemplary sequences for CDR-1 of the light chain variable region include RASKSVSTSGYSYMH (SEQ ID NO:21) and RASQSISNNLH (SEQ ID NO:22). Exemplary amino acid sequences for CDR-2 of the light chain variable region include LVSNLES (SEQ ID NO:23) and YASQSIS (SEQ ID NO:73). Exemplary amino acid sequences for CDR-3 of the light chain variable region include QHIRELT (SEQ ID NO:24) and QQINSWPLT (SEQ ID NO:25). In some forms, the first, second, and third CDR regions of the light chain variable region include variant amino acid sequences having about 70%, about 80%, about 90%, about 95%, about 96%, about 97%, about 98%, about 99% sequence identity to SEQ ID NO:21, SEQ ID NO:22, SEQ ID NO:23, SEQ ID NO:24, SEQ ID NO:25, and SEQ ID NO:73 respectively.

The framework region (FR) regions of the light chain variable region are shown in underlining in SEQ ID NO:19 and SEQ ID NO:20, including a first, second, third, and fourth FR regions, FR1, FR2, FR3, and FR4 respectively. Exemplary amino acid sequences for FR1 of the light chain variable region include DIVLTQSPASLAVSLGQRATISY (SEQ ID NO:26) and DIVLTQSPATLSVTPGDSVSLSC (SEQ ID NO:27). Exemplary sequences for FR2 of the light chain variable region includes WNQQKPGQPPRLLIY (SEQ ID NO:28) and WYQQKSHESPRLLIK (SEQ ID NO:29). Exemplary sequences for FR3 of the light chain variable region include GVPARFSGSGSGTDFTLNIHPVEEEDAATYYC (SEQ ID NO:30) and GIPSRFSGSGSGTDFTLSINSVETEDFGMYFC (SEQ ID NO:31). Exemplary sequences for FR4 of the light chain variable region include FGGGTKLEIK (SEQ ID NO:32) and FGAGTKLELK (SEQ ID NO:33). In some forms, the first, second, third, and fourth FR regions of the light chain variable region include variant amino acid sequences having about 70%, about 80%, about 90%, about 95%, about 96%, about 97%, about 98%, about 99% sequence identity to SEQ ID NO:26, SEQ ID NO:27, SEQ ID NO:28, SEQ ID NO:29, SEQ ID NO:30, SEQ ID NO:31, SEQ ID NO:32, and/or SEQ ID NO:33 respectively.

The signal peptides for the light chain variable region are shown in italic in SEQ ID NO:19 and SEQ ID NO:20. Exemplary sequences for the signal peptide of the heavy chain variable region include MGTAALGSRFHW (SEQ ID NO:34) and MVFTPQILGLMLEWISASRG (SEQ ID NO:35). In some forms, the signal peptide of the light chain variable region includes variant amino acid sequences having about 70%, about 80%, about 90%, about 95%, about 96%, about 97%, about 98%, about 99% sequence identity to SEQ ID NO:34 or SEQ ID NO:35.

In some forms, the constant region of the light chain variable region can include the amino acid sequence:

(SEQ ID NO: 37)
RADAAPTVSIFPPSSEQLTSGGASVVCFLNNFYPKDINVKWKIDGSERQ
NGVLNSWTDQDSKDSTYSMSSTLTLTKDEYERHNSYTCEATHKTSTSPI
VKSFNRNEC.

In some forms, the constant region of the light chain variable region includes variant amino acid sequences having about 70%, about 80%, about 90%, about 95%, about 96%, about 97%, about 98%, about 99% sequence identity to SEQ ID NO:37.

### 3. Exemplary LANCL1 Monoclonal Antibodies

In some forms, the disclosed monoclonal antibodies have the following sequences, with the CDR's indicated in bold italic font, and the FR's indicated by underlining:

### (i) 4D9:

VH:
(SEQ ID NO: 3)
MGWSWIFLFLLSGTAGVLSEVQLQQSGPELVKPGASVKISCKASGYTFTDYYMNWVKQS
HGKSLEWIGDINPNNGGASYNQKFKGKATLTVDKSSSTAYMELRSLTSEDSAVYYCVRS
GDGYYFASWGQGTLLTVSAAKTTPPSVYPLAPGCGDTTGSSVTLGCLVKGYFPESVTVT
WNSGSLSSSVHTFPALLQSGLYTMSSSVTVPSSTWPSQTVTCSVAHPASSTTVDKKLEP
SGPISTINPCPPCKECHKCPAPNLEGGPSVFIFPPNIKDVLMISLTPKVTCVVVDVSED
DPDVRISWFVNNVEVHTAQTQTHREDYNSTIRVVSALPIQHQDWMSGKEFKCKVNNKDL
PSPIERTISKIKGLVRAPQVYILPPPAEQLSRKDVSLTCLVVGFNPGDISVEWTSNGHT
EENYKDTAPVLDSDGSYFIYSKLDIKTSKWEKTDSFSCNVRHEGLKNYYLKKTISRSPG
K-;
The CDR-1 is
(SEQ ID NO: 5)
DYYMN,
the CDR-2 is
(SEQ ID NO: 6)
DINPNNGGASYNQKFKG,
and
the CDR-3 is
(SEQ ID NO: 8)
SGDGYYFAS.
The FR1 is
(SEQ ID NO: 10)
EVQLQQSGPELVKPGASVKISCKASGYTFT;
the FR2 is
(SEQ ID NO: 12)
WVKQSHGKSLEWIG,
the FR3 is
(SEQ ID NO: 13)
KATLTVDKSSSTAYMELRSLTSEDSAVYYCVR,
and
the FR4 is
(SEQ ID NO: 15)
WGQGTLLTVSA.
VL:
(SEQ ID NO: 19)
MGTAALGSRFHWDIVLTQSPASLAVSLGQRATISYRASKSVSTSGYSYMHWNQQKPGQP
PRLLIYLVSNLESGVPARFSGSGSGTDFTLNIHPVEEEDAATYYCQHIRELTFGGGTKL
EIKRADAAPTVSIFPPSSEQLTSGGASVVCFLNNFYPKDINVKWKIDGSERQNGVLNSW
TDQDSKDSTYSMSSTLTLTKDEYERHNSYTCEATHKTSTSPIVKSFNRNEC-.
The CDR-1 is
(SEQ ID NO: 21)
RASKSVSTSGYSYMH,
the CDR-2 is
(SEQ ID NO: 23)
LVSNLES,
and
the CDR-3 is
(SEQ ID NO: 24)
QHIRELT.
The FR1 is
(SEQ ID NO: 26)
DIVLTQSPASLAVSLGORATISY;
the FR2 is
(SEQ ID NO: 28)
WNQQKPGQPPRLLIY,
the FR3 is
(SEQ ID NO: 30)
GVPARFSGSGSGTDFTLNIHPVEEEDAATYYC,
and
the FR4 is
(SEQ ID NO: 32)
FGGGTKLEIK.

### (ii) 19G4:

VH:
(SEQ ID NO: 4)
MGWSWIFLFLLSGTAGVHSEVQLQQSGPVLVKPGASVKMSCKASGYTFTDYYMNWVKQS
HGKSLEWIGVINPYNGHTNYNQKFKGKATLTVDKSSNTAYMELNSLTSEDSAVYYCARF
PYYGSSYRVDYWGQGTTLTVSSAKTTPPSVYPLAPGCGDTTGSSVTLGCLVKGYFPESV
TVTWNSGSLSSSVHTFPALLQSGLYTMSSSVTVPSSTWPSQTVTCSVAHPASSTTVDKK
LEPSGPISTINPCPPCKECHKCPAPNLEGGPSVFIFPPNIKDVLMISLTPKVTCVVVDV
SEDDPDVRISWFVNNVEVHTAQTQTHREDYNSTIRVVSALPIQHQDWMSGKEFKCKVNN
KDLPSPIERTISKIKGLVRAPQVYILPPPAEQLSRKDVSLTCLVVGFNPGDISVEWTSN
GHTEENYKDTAPVLDSDGSYFIYSKLDIKTSKWEKTDSFSCNVRHEGLKNYYLKKT
ISRSPGK-.
The CDR-1 is
(SEQ ID NO: 5)
DYYMN,
the CDR-2 is
(SEQ ID NO: 7)
VINPYNGHTNYNQKFKG,
and
the CDR-3 is
(SEQ ID NO: 9)
FPYYGSSYRVDY.
The FR1 is
(SEQ ID NO: 11)
EVQLQQSGPVLVKPGASVKMSCKASGYTFT,
the FR2 is
(SEQ ID NO: 12)
WVKQSHGKSLEWIG,
the FR3 is
(SEQ ID NO: 14)
KATLTVDKSSNTAYMELNSLTSEDSAVYYCAR,
and
the FR4 is
(SEQ ID NO: 16)
WGQGTTLTVSS.
VL:
(SEQ ID NO: 20)
MVFTPQILGLMLEWISASRGDIVLTQSPATLSVTPGDSVSLSCRASQSISNNLHWYQQK
SHESPRLLIKYASQSISGIPSRFSGSGSGTDFTLSINSVETEDFGMYFCQQINSWPLTF
GAGTKLELKRADAAPTVSIFPPSSEQLTSGGASVVCFLNNFYPKDINVKWKIDGSERQN
GVLNSWTDQDSKDSTYSMSSTLTLTKDEYERHNSYTCEATHKTSTSPIVKSFNRNEC-.
The CDR-1 is
(SEQ ID NO: 22)
RASQSISNNLH,
the CDR-2 is
(SEQ ID NO: 73)
YASQSIS,
and
the CDR-3 is
(SEQ ID NO: 25)
QQINSWPLT.
The FR1 is
(SEQ ID NO: 27)
DIVLTQSPATLSVTPGDSVSLSC,
the FR2 is
(SEQ ID NO: 29)
WYQQKSHESPRLLIK,
the FR3 is
(SEQ ID NO: 31)
GIPSRFSGSGSGTDFTLSINSVETEDFGMYFC,
and
FR4 is
(SEQ ID NO: 33)
FGAGTKLELK.

### B. Chimeric and Humanized Antibodies

In some forms, the anti-LANCL1 antibodies can be a chimeric antibody or a humanized antibody. Constant regions need not be present, but if they are, are typically substantially identical to human immunoglobulin constant regions, i.e., at least about 85-90%, preferably about 95% or more identical. Hence, all parts of a humanized immunoglobulin, except possibly the CDR's, are substantially identical to corresponding parts of natural human immunoglobulin sequences. A humanized antibody is an antibody having a humanized light chain and a humanized heavy chain immunoglobulin. For example, a humanized antibody would not encompass a typical chimeric antibody, because, e.g., the entire variable region of a chimeric antibody is non-human. One says that the donor antibody has been “humanized,” by the process of “humanization,” because the resultant humanized antibody is expected to bind to the same antigen as the donor antibody that provides the CDR's.

For the most part, humanized antibodies are human immunoglobulins (recipient antibody) in which hypervariable region residues of the recipient are replaced by hypervariable region residues from a non-human species (donor antibody) such as mouse, rat, rabbit or a non-human primate having the desired specificity, affinity, and capacity. In some instances, Framework Region (FR) residues of the human immunoglobulin are replaced by corresponding non-human residues. Furthermore, humanized antibodies can include residues which are not found in the recipient antibody or in the donor antibody. These modifications are made to further refine antibody performance. In general, the humanized antibody will include substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will include at least a portion of an immunoglobulin constant region (Fc), typically that of a human immunoglobulin that immunospecifically binds to an FcγRIIB polypeptide, that has been altered by the introduction of amino acid residue substitutions, deletions or additions (i.e., mutations). See also, e.g., European Patent Nos. EP 239,400, EP 592,106, and EP 519,596; International Publication Nos. WO 91/09967 and WO 93/17105; U.S. Pat. Nos. 5,225,539, 5,530,101, 5,565,332, 5,585,089, 5,766,886, and 6,407,213; and Padlan, 1991, Molecular Immunology 28(4/5):489-498; Studnicka et al., 1994, Protein Engineering 7(6):805-814; Roguska et al., 1994, PNAS 91:969-973; Tan et al., 2002, J. Immunol. 169:1119-1125; Caldas et al., 2000, Protein Eng. 13:353-360; Morea et al., 2000, Methods 20:267-79; Baca et al., 1997, J. Biol. Chem. 272:10678-10684; Roguska et al., 1996, Protein Eng. 9:895-904; Couto et al., 1995, Cancer Res. 55 (23 Supp): 5973s-5977s; Couto et al., 1995, Cancer Res. 55:1717-22; Sandhu, 1994, Gene 150:409-10; Pedersen et al., 1994, J. Mol. Biol. 235:959-973; Jones et al., 1986, Nature 321:522-525; Reichmann et al., 1988, Nature 332:323-329; and Presta, 1992, Curr. Op. Struct. Biol. 2:593-596).

DNA sequences coding for preferred human acceptor framework sequences include but are not limited to FR segments from the human germline VH segment VH1-18 and JH6 and the human germline VL segment VK-A26 and JK4. In a specific embodiment, one or more of the CDRs are inserted within framework regions using routine recombinant DNA techniques. The framework regions can be naturally occurring or consensus framework regions, and preferably human framework regions (see, e.g., Chothia et al., 1998, “Structural Determinants In The Sequences Of Immunoglobulin Variable Domain,” J. Mol. Biol. 278:457-479 for a listing of human framework regions).

A humanized or chimeric antibody can include substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence.

In some forms, heavy chain variable region (VH) CDR-1 of the chimeric and/or humanized antibody is DYYMN (SEQ ID NO:5). In some forms, VH CDR-2 of the chimeric or humanized antibody is DINPNNGGASYNQKFKG (SEQ ID NO:6) or VINPYNGHTNYNQKFKG (SEQ ID NO:7). In some forms, the VH CDR-3 of the chimeric or humanized antibody is SGDGYYFAS (SEQ ID NO:8) or FPYYGSSYRVDY (SEQ ID NO:9). In some forms, the first, second, and third VH CDR regions are variant amino acid sequences having about 70%, about 80%, about 90%, about 95%, about 96%, about 97%, about 98%, about 99% sequence identity to SEQ ID NO:5, SEQ ID NO:6, SEQ ID NO:7, SEQ ID NO:8, or SEQ ID NO:9.

In some forms, light chain variable region (VL) CDR-1 of the chimeric or humanized antibody is RASKSVSTSGYSYMH (SEQ ID NO:21) or RASQSISNNLH (SEQ ID NO:22). In some forms, VL CDR-2 of the chimeric or humanized antibody is LVSNLES (SEQ ID NO:23) or YASQSIS (SEQ ID NO:73). In some forms, VL CDR-3 of the chimeric or humanized antibody is QHIRELT (SEQ ID NO:24) and QQINSWPLT (SEQ ID NO:25). In some forms, the first, second, and third VL CDR regions of the chimeric or humanized antibody are variant amino acid sequences having about 70%, about 80%, about 90%, about 95%, about 96%, about 97%, about 98%, about 99% sequence identity to SEQ ID NO:21, SEQ ID NO:22, SEQ ID NO:23, SEQ ID NO:24, SEQ ID NO:73 or SEQ ID NO:25.

In some forms the heavy chain variable region (VH) of the chimeric humanized anti-LANCL1 monoclonal antibody (19G4) has the sequence: EVQLQQSGPVLVKPGASVKMSCKASGYTFTDYYMNWVKQSHGKSLEWIGVINP YNGHTNYNQKFKGKATLTVDKSSNTAYMELNSLTSEDSAVYYCARFPYYGSSY RVDYWGQGTTLTVSS (SEQ ID NO:75). In some forms, the heavy chain variable region (VH) of the chimeric humanized anti-LANCL1 monoclonal antibody (19G4) are variant amino acid sequences having about 70%, about 80%, about 90%, about 95%, about 96%, about 97%, about 98%, about 99% sequence identity to SEQ ID NO:75. In some forms the variant of SEQ ID NO:75 has the sequence QVQLVQSGAEVKKPGASVKVSCKASGYTFTDYYMNWVRQAPGQGLEWIGVIN PYNGHTNYNQKFKGRVTMTVDTSTSTVYMELSSLRSEDTAVYYCARFPYYGSS YRVDYWGQGTTVTVSS (SEQ ID NO:77) (VH1). In some forms the variant of SEQ ID NO:75 has the sequence QVQLVQSGAEVKKPGASVKVSCKASGYTFTDYYMNWVRQAPGQGLEWIGVIN PYNGHTNYNQKFKGRATMTVDTSTSTVYMELSSLRSEDTAVYYCARFPYYGSS YRVDYWGQGTTVTVSS (SEQ ID NO:78) (VH2). In some forms the variant of SEQ ID NO:75 has the sequence QVQLVQSGAEVKKPGASVKVSCKASGYTFTDYYMNWVKQAPGQGLEWIGVIN PYNGHTNYNQKFKGRATMTVDKSTSTVYMELSSLRSEDTAVYYCARFPYYGSS YRVDYWGQGTTVTVSS (SEQ ID NO:79) (VH3).

In some forms the light chain variable region (VL) of the chimeric humanized anti-LANCL1 monoclonal antibody (19G4) has the sequence: DIVLTQSPATLSVTPGDSVSLSCRASQSISNNLHWYQQKSHESPRLLIKYASQSIS GIPSRESGSGSGTDFTLSINSVETEDFGMYFCQQINSWPLTFGAGTKLELK (SEQ ID NO:76). In some forms, the light chain variable region (VL) of the chimeric humanized anti-LANCL1 monoclonal antibody (19G4) are variant amino acid sequences having about 70%, about 80%, about 90%, about 95%, about 96%, about 97%, about 98%, about 99% sequence identity to SEQ ID NO:76. In some forms the variant of SEQ ID NO:76 has the sequence EIVMTQSPATLSVSPGERATLSCRASQSISNNLHWYQQKPGQAPRLLIKYASQSIS GIPARESGSGSGTEFTLTISSLQSEDFAVYYCQQINSWPLTFGGGTKLEIK (SEQ ID NO:80) (VL1). In some forms the variant of SEQ ID NO:76 has the sequence EIVMTQSPATLSVSPGERATLSCRASQSISNNLHWYQQKPGQAPRLLIKYASQSIS GIPSRESGSGSGTEFTLTISSLQSEDFAVYYCQQINSWPLTFGGGTKLEIK (SEQ ID NO:81) (VL.2). In some forms the variant of SEQ ID NO:76 has the sequence EIVMTQSPATLSVSPGERATLSCRASQSISNNLHWYQQKPGQAPRLLIKYASQSIS GIPSRESGSGSGTEFTLTISSLQSEDFAVYFCQQINSWPLTFGGGTKLEIK (SEQ ID NO:82) (VL3).

Preferably, the antibody also includes at least a portion of an immunoglobulin constant region (Fc), typically that of a human immunoglobulin. The constant domains of the antibodies can be selected with respect to the proposed function of the antibody, in particular the effector function which can be required. In some embodiments, the constant domains of the antibodies are (or include) human IgA, IgD, IgE, IgG or IgM domains. In a specific embodiment, human IgG constant domains, especially of the IgG1 and IgG3 isotypes are used, when the humanized antibodies are intended for therapeutic uses and antibody effector functions such as antibody-dependent cell-mediated cytotoxicity (ADCC) and complement-dependent cytotoxicity (CDC) activity are needed. In alternative embodiments, IgG2 and IgG4 isotypes are used when the antibody is intended for therapeutic purposes and antibody effector function is not required. The disclosure encompasses Fc constant domains including one or more amino acid modifications which alter antibody effector functions such as those disclosed in U.S. Patent Application Publication Nos. 2005/0037000 and 2005/0064514.

In some embodiments, the antibody contains both the light chain as well as at least the variable domain of a heavy chain. In other embodiments, the antibody can further include one or more of the CH1, hinge, CH2, CH3, and CH4 regions of the heavy chain. The antibody can be selected from any class of immunoglobulins, including IgM, IgG, IgD, IgA and IgE, and any isotype, including IgG1, IgG2, IgG3 and IgG4. In some embodiments, the constant domain is a complement fixing constant domain where it is desired that the antibody exhibits cytotoxic activity, and the class is typically IgG1. In other embodiments, where such cytotoxic activity is not desirable, the constant domain can be of the IgG2 class. The antibody can include sequences from more than one class or isotype, and selecting particular constant domains to optimize desired effector functions is within the ordinary skill in the art. In some embodiments, the antibody is not a mouse IgG1 or a mouse IgG2a.

The framework and CDR regions of a humanized antibody need not correspond precisely to the parental sequences, e.g., the donor CDR or the consensus framework can be mutagenized by substitution, insertion or deletion of at least one residue so that the CDR or framework residue at that site does not correspond to either the consensus or the donor antibody. Such mutations, however, are preferably not extensive. Usually, at least 75% of the humanized antibody residues will correspond to those of the parental framework region (FR) and CDR sequences, more often 90%, and most preferably greater than 95%. Humanized antibodies can be produced using variety of techniques known in the art, including, but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7 (6): 805-814; and Roguska et al., 1994, Proc. Natl. Acad. Sci. 91:969-973), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., 2002, J. Immunol. 169:1119-25, Caldas et al., 2000, Protein Eng. 13:353-60, Morea et al., 2000, Methods 20:267-79, Baca et al., 1997, J. Biol. Chem. 272:10678-84, Roguska et al., 1996, Protein Eng. 9:895-904, Couto et al., 1995, Cancer Res. 55 (23 Supp): 5973s-5977s, Couto et al., 1995, Cancer Res. 55:1717-22, Sandhu, 1994, Gene 150:409-10, Pedersen et al., 1994, J. Mol. Biol. 235:959-73, Jones et al., 1986, Nature 321:522-525, Riechmann et al., 1988, Nature 332:323, and Presta, 1992, Curr. Op. Struct. Biol. 2:593-596. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; U.S. Publication Nos. 2004/0049014 and 2003/0229208; U.S. Pat. Nos. 6,350,861; 6,180,370; 5,693,762; 5,693,761; 5,585,089; and 5,530,101 and Riechmann et al., 1988, Nature 332:323).

### C. Derivatives and Conjugates

The disclosure particularly contemplates the production and use of derivatives of any of the above-described antibodies and their antigen-binding fragments. The term derivative encompasses an antibody or antigen-binding fragment thereof that specifically binds to an antigen, but which includes, one, two, three, four, five or more amino acid substitutions, additions, deletions or modifications relative to a “parental” (or wild-type) molecule (also referred to as variants). Such amino acid substitutions or additions can introduce naturally occurring (i.e., DNA-encoded) or non-naturally occurring amino acid residues.

The term derivative also encompasses, for example, chimeric or humanized variants of any of the disclosed antibodies, as well as variants having altered CH1, hinge, CH2, CH3 or CH4 regions, so as to form, for example antibodies, etc., having variant Fc regions that exhibit enhanced or impaired effector or binding characteristics.

The term derivative additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity.,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1-6) Dextran Increases Its Affinity For Antigen.” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8): 847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity.,” J. Biol. Chem. 277(30): 26733-26740).

In some embodiments, a humanized antibody is a derivative. Such a humanized antibody includes amino acid residue substitutions, deletions or additions in one or more non-human CDRs. The humanized antibody derivative can have substantially the same binding, better binding, or worse binding when compared to a non-derivative humanized antibody. In specific embodiments, one, two, three, four, or five amino acid residues of the CDRs have been substituted, deleted or added (i.e., mutated).

A derivative antibody or antibody fragment can be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

Derivatized antibodies can be used to alter the half-lives (e.g., serum half-lives) of parental antibodies in a mammal, preferably a human. Preferably such alteration will result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than months. The increased half-lives of the humanized antibodies of the present disclosure or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor. The humanized antibodies can be engineered to increase biological half-lives (see, e.g. U.S. Pat. No. 6,277,375). For example, humanized antibodies can be engineered in the Fc-hinge domain to have increased in vivo or serum half-lives.

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The antibodies can also be modified by the methods and coupling agents described by Davis et al. (See U.S. Pat. No. 4,179,337) in order to provide compositions that can be injected into the mammalian circulatory system with substantially no immunogenic response.

One embodiment encompasses modification of framework residues of humanized anti-LANCL1 antibodies. Framework residues in the framework regions can be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann, L. et al. (1988) “Reshaping Human Antibodies For Therapy,” Nature 332:323-327).

Yet another embodiment encompasses anti-LANCL1 antibodies (and more preferably, humanized antibodies) and antigen-binding fragments thereof that are recombinantly fused or chemically conjugated (including both covalently and non-covalently conjugations) to a heterologous molecule (i.e., an unrelated molecule). The fusion does not necessarily need to be direct but can occur through linker sequences.

In one embodiment such heterologous molecules are polypeptides having at least 10, at least 20, at least 30, at least 40, at least 50, at least 60, at least 70, at least 80, at least 90 or at least 100 amino acids. Such heterologous molecules can alternatively be enzymes, hormones, cell surface receptors, drug moieties, such as: toxins (such as abrin, ricin A, pseudomonas exotoxin (i.e., PE-40), diphtheria toxin, ricin, gelonin, or pokeweed antiviral protein), proteins (such as tumor necrosis factor, interferon (e.g., α-interferon, β-interferon), nerve growth factor, platelet derived growth factor, tissue plasminogen activator, or an apoptotic agent (e.g., tumor necrosis factor-α, tumor necrosis factor-β)), biological response modifiers (such as, for example, a lymphokine (e.g., interleukin-1 (“IL-1”), interleukin-2 (“IL-2”), interleukin-6 (“IL-6”)), granulocyte macrophage colony stimulating factor (“GM-CSF”), granulocyte colony stimulating factor (“G-CSF”), or macrophage colony stimulating factor, (“M-CSF”)), or growth factors (e.g., growth hormone (“GH”))), cytotoxins (e.g., a cytostatic or cytocidal agent, such as paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof), antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, BiCNU® (carmustine; BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), or anti-mitotic agents (e.g., vincristine and vinblastine).

Techniques for conjugating such therapeutic moieties to antibodies are well known; see, e.g., Arnon et al., “Monoclonal Antibodies For Immunotargeting Of Drugs In Cancer Therapy”, in MONOCLONAL ANTIBODIES AND CANCER THERAPY, Reisfeld et al. (eds.), 1985, pp. 243-56, Alan R. Liss, Inc.); Hellstrom et al., “Antibodies For Drug Delivery”, in CONTROLLED DRUG DELIVERY (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in MONOCLONAL ANTIBODIES '84: BIOLOGICAL AND CLINICAL APPLICATIONS, Pinchera et al. (eds.), 1985, pp. 475-506); “Analysis, Results, And Future Prospective Of The Therapeutic Use Of Radiolabeled Antibody In Cancer Therapy”, in MONOCLONAL ANTIBODIES FOR CANCER DETECTION AND THERAPY, Baldwin et al. (eds.), 1985, pp. 303-16, Academic Press; and Thorpe et al. (1982) “The Preparation And Cytotoxic Properties Of Antibody-Toxin Conjugates,” Immunol. Rev. 62:119-158.

In one embodiment, the anti-LANCL1 antibodies or fusion molecules include an Fc portion. The Fc portion of such molecules can be varied by isotype or subclass, can be a chimeric or hybrid, and/or can be modified, for example to improve effector functions, control of half-life, tissue accessibility, augment biophysical characteristics such as stability, and improve efficiency of production (and less costly). Many modifications useful in construction of disclosed fusion proteins and methods for making them are known in the art, see for example Mueller, J. P. et al. (1997) “Humanized Porcine VCAM-Specific Monoclonal Antibodies With Chimeric IgG2/G4 Constant Regions Block Human Leukocyte Binding To Porcine Endothelial Cells,” Mol. Immun. 34(6):441-452, Swann, P. G. (2008) “Considerations For The Development Of Therapeutic Monoclonal Antibodies,” Curr. Opin. Immun. 20:493-499 (2008), and Presta, L. G. (2008) “Molecular Engineering And Design Of Therapeutic Antibodies,” Curr. Opin. Immun. 20:460-470. In some embodiments the Fc region is the native IgG1, IgG2, or IgG4 Fc region. In some embodiments the Fc region is a hybrid, for example a chimeric having IgG2/IgG4 Fc constant regions. Modifications to the Fc region include, but are not limited to, IgG4 modified to prevent binding to Fc gamma receptors and complement, IgG1 modified to improve binding to one or more Fc gamma receptors, IgG1 modified to minimize effector function (amino acid changes), IgG1 with altered/no glycan (typically by changing expression host), and IgG1 with altered pH-dependent binding to FcRn, and IgG4 with serine at amino acid resident #228 in the hinge region changed to proline (S228P) to enhance stability. The Fc region can include the entire hinge region, or less than the entire hinge region.

For example, the therapeutic outcome in patients treated with rituximab (a chimeric mouse/human IgG1 monoclonal antibody against CD20) for non-Hodgkin's lymphoma or Waldenstrom's macroglobulinemia correlated with the individual's expression of allelic variants of Fcγ receptors with distinct intrinsic affinities for the Fc domain of human IgG1. In another example, patients with high affinity alleles of the low affinity activating Fc receptor CD16A (FcγRIIIA) showed higher response rates and, in the cases of non-Hodgkin's lymphoma, improved progression-free survival. In another example, the Fc domain can contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA).

Another embodiment includes IgG₂₋₄ hybrids and IgG₄ mutants that have reduce binding to FcR which increases their half-life. Representative IG₂₋₄ hybrids and IgG4 mutants are described in Angal, S. et al. (1993) “A Single Amino Acid Substitution Abolishes the Heterogeneity Of Chimeric Mouse/Human (Igg4) Antibody,” Molec. Immunol. 30(1):105-108; Mueller, J. P. et al. (1997) “Humanized Porcine VCAM-Specific Monoclonal Antibodies with Chimeric Igg2/G4 Constant Regions Block Human Leukocyte Binding To Porcine Endothelial Cells,” Mol. Immun. 34(6):441-452; and U.S. Pat. No. 6,982,323. In some embodiments the IgG₁ and/or IgG₂ domain is deleted for example, Angal, s. et al. describe IgG₁ and IgG₂ having serine 241 replaced with a proline.

Substitutions, additions or deletions in the derivatized antibodies can be in the Fc region of the antibody and can thereby serve to modify the binding affinity of the antibody to one or more FcγR. Methods for modifying antibodies with modified binding to one or more FcγR are known in the art, see, e.g., PCT Publication Nos. WO 04/029207, WO 04/029092, WO 04/028564, WO 99/58572, WO 99/51642, WO 98/23289, WO 89/07142, WO 88/07089, and U.S. Pat. Nos. 5,843,597 and 5,642,821. In one particular embodiment, the modification of the Fc region results in an antibody with an altered antibody-mediated effector function, an altered binding to other Fc receptors (e.g., Fc activation receptors), an altered antibody-dependent cell-mediated cytotoxicity (ADCC) activity, an altered C1q binding activity, an altered complement-dependent cytotoxicity activity (CDC), a phagocytic activity, or any combination thereof.

In some forms, the disclosure encompasses antibodies whose Fc region will have been modified so that the molecule will exhibit altered Fc receptor (FcR) binding activity, for example to exhibit decreased activity toward activating receptors such as FcγRIIA or FcγRIIIA, or increased activity toward inhibitory receptors such as FcγRIIB. Preferably, such antibodies will exhibit decreased antibody-dependent cell-mediated cytotoxicity (ADCC) or complement dependent cytotoxicity (CDC) activities (relative to a wild-type Fc receptor).

Modifications that affect Fc-mediated effector function are well known in the art (see U.S. Pat. No. 6,194,551, and WO 00/42072; Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fegamma Receptors,” Cancer Res. 57(18):8882-8890; Shields, R. L. et al. (2001) “High Resolution Mapping of the Binding Site on Human IgG1 for FcγRI, FcγRII, FcγRIII, and FcRn and Design of IgG1 Variants with Improved Binding to the FcγR,” J. Biol. Chem. 276(9):6591-6604). Exemplary variants of human IgG1 Fc domains with reduced binding to FcγRIIA or FcγRIIIA, but unchanged or enhanced binding to FcγRIIB, include S239A, H268A, S267G, E269A, E293A, E293D, Y296F, R301A, V303A, A327G, K322A, E333A, K334A, K338A, A339A, D376A.

In some embodiments, the disclosure encompasses antibodies whose Fc region will have been deleted (for example, a Fab or F(ab)₂, etc.).

Any of the molecules of the present disclosure can be fused to marker sequences, such as a peptide, to facilitate purification. In preferred embodiments, the marker amino acid sequence is a hexa-histidine peptide, the hemagglutinin “HA” tag, which corresponds to an epitope derived from the influenza hemagglutinin protein (Wilson, I. A. et al. (1984) “The Structure Of An Antigenic Determinant In A Protein,” Cell, 37:767-778) and the “flag” tag (Knappik, A. et al. (1994) “An Improved Affinity Tag Based On The FLAG Peptide For The Detection And Purification Of Recombinant Antibody Fragments,” Biotechniques 17(4):754-761).

The present disclosure also encompasses antibodies or their antigen-binding fragments that are conjugated to a diagnostic or therapeutic agent or any other molecule for which serum half-life is desired to be increased. The antibodies can be used diagnostically (in vivo, in situ or in vitro) to, for example, monitor the development or progression of a disease, disorder or infection as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance can be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present disclosure. Such diagnosis and detection can be accomplished by coupling the antibody to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

The molecules of the present disclosure can be conjugated to a second antibody to form an antibody heteroconjugate as described by Segal in U.S. Pat. No. 4,676,980. Such heteroconjugate antibodies may additionally bind to haptens (such as fluorescein, etc.), or to cellular markers, or to cytokines, or chemokines (e.g., CCL21), etc.

The molecules of the present disclosed can be attached to solid supports, which are particularly useful for immunoassays or purification of the target antigen or of other molecules that are capable of binding to target antigen that has been immobilized to the support via binding to an antibody or antigen-binding fragment of the present disclosure. Such solid supports include, but are not limited to, glass, cellulose, polyacrylamide, nylon, polystyrene, polyvinyl chloride or polypropylene.

### D. CAR Proteins

Chimeric Antigen Receptor (CAR) proteins including the disclosed anti-LANCL1 proteins as an antigen binding domain, and cells expressing the same are also provided. Typically, CARs also include a transmembrane domain and one or more intracellular/cytoplasmic domains.

CARs are engineered receptors that possess both antigen-binding and T-cell-activating functions. Immunotherapy using T cells genetically engineered to express a CAR is rapidly emerging as a promising new treatment for hematological and non-hematological malignancies. Based on the location of the CAR in the membrane of the cell, the CAR can be divided into three main distinct domains, including an extracellular antigen-binding domain, followed by a space region, a transmembrane domain, and the intracellular signaling domain. The antigen-binding domain, most commonly derived from variable regions of immunoglobulins, typically contains VH and VL chains that are joined up by a linker to form the so-called “scFv.” The segment interposing between the antigen-binding domain (e.g., scFv) and the transmembrane domain is a “spacer domain.” The spacer domain can include the constant IgG1 hinge-CH2-CH3 Fc domain. In some cases, the spacer domain and the transmembrane domain are derived from CD8. The intracellular signaling domains mediating T cell activation can include a CD35 co-receptor signaling domain derived from C-region of the TCR a and B chains and one or more costimulatory domains.

In the disclosed CARs, the antigen binding domain is typically a disclosed anti-HERV-K Env binding protein. In some forms, the antigen-binding domain is derived from an antibody, e.g., a disclosed Kenv antibody. As introduced above, the term antibody herein refers to natural or synthetic polypeptides that bind a target antigen, and such antibodies can form part or all of the antigen binding domain of the CAR. The term includes polyclonal and monoclonal antibodies, including intact antibodies and functional (e.g., antigen-binding) antibody fragments, including Fab fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. The term also encompasses intact or full-length antibodies, including antibodies of any class or subclass, including IgG and sub-classes thereof, IgM, IgE, IgA, and IgD. The antigen-binding domain of a CAR can contain complementary determining regions (CDR) of an antibody, variable regions of an antibody, and/or antigen binding fragments thereof. For example, the antigen-binding domain for an HERV-K Env CAR can be derived from a disclosed Kenv antibody as described above. In some forms, the antigen-binding domain can include an F(ab′)2, Fab′, Fab, Fv or scFv.

In some forms, the CAR includes one or more spacer domain(s) (also referred to as hinge domain) that is located between the extracellular antigen-binding domain and the transmembrane domain. A spacer domain is an amino acid segment that is generally found between two domains of a protein and may allow for flexibility of the protein and movement of one or both of the domains relative to one another. Any amino acid sequence that provides such flexibility and movement of the extracellular antigen-binding domain relative to the transmembrane domain can be used. The spacer domain can be a spacer or hinge domain of a naturally occurring protein. In some forms, the hinge domain is derived from CD8a, such as, a portion of the hinge domain of CD8a, e.g., a fragment containing at least 5 (e.g., 5, 10, 15, 20, 25, 30, 35, or 40) consecutive amino acids of the hinge domain of CD8a. Hinge domains of antibodies, such as an IgG, IgA, IgM, IgE, or IgD antibodies can also be used. In some forms, the hinge domain is the hinge domain that joins the constant CH1 and CH2 domains of an antibody. Non-naturally occurring peptides may also be used as spacer domains. For example, the spacer domain can be a peptide linker, such as a (G×S)n linker, wherein x and n, independently can be an integer of 3 or more, including 3, 4, 5, 6, 7, 8, 9, 10, 11, 12, or more.

In some forms, the CAR includes a transmembrane domain that can be directly or indirectly fused to the antigen-binding domain. The transmembrane domain may be derived either from a natural or a synthetic source. In some forms, the transmembrane domain of the CAR includes a transmembrane domain of an alpha, beta or zeta chain of a T-cell receptor, CD8, CD4, CD28, CD137, CD80, CD86, CD152 (CTLA-4) or PD1, or a portion thereof. Transmembrane domains can also contain at least a portion of a synthetic, non-naturally occurring protein segment. In some forms, the transmembrane domain is a synthetic, non-naturally occurring alpha helix or beta sheet. In some forms, the protein segment is at least about 15 amino acids, e.g., at least 15, 16, 17, 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776.

The intracellular signaling domain is responsible for activation of at least one of the normal effector functions of the immune effector cell expressing the CAR. The term effector function refers to a specialized function of a cell. Effector function of a T cell, for example, may be cytolytic activity or helper activity including the secretion of cytokines. In some forms, an intracellular signaling domain includes the zeta chain of the T cell receptor or any of its homologs (e.g., eta, delta, gamma or epsilon), MBI chain, B29, Fc RIII, Fc RI and combinations of signaling molecules such as CD3ζ and CD28, 4-1BB, OX40 and combination thereof, as well as other similar molecules and fragments. Intracellular signaling portions of other members of the families of activating proteins can be used, such as FcγRIII and FcεRI.

Many immune effector cells require co-stimulation, in addition to stimulation of an antigen-specific signal, to promote cell proliferation, differentiation and survival, as well as to activate effector functions of the cell. Therefore, in some forms, the CAR includes at least one co-stimulatory signaling domain. The term co-stimulatory signaling domain, refers to at least a portion of a protein that mediates signal transduction within a cell to induce an immune response such as an effector function. The co-stimulatory signaling domain can be a cytoplasmic signaling domain from a co-stimulatory protein, which transduces a signal and modulates responses mediated by immune cells, such as T cells, NK cells, macrophages, neutrophils, or eosinophils. In some forms, the co-stimulatory signaling domain is derived from a co-stimulatory molecule selected from CD27, CD28, CD137, 0X40, CD30, CD40, CD3, LFA-1, ICOS, CD2, CD7, LIGHT, NKG2C, B7-H3, ligands of CD83 and combinations thereof.

CARs can be used in order to generate immuno-responsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and PCT Publication WO 9215322, each of which is specifically incorporated by reference herein in its entirety). Alternative CAR constructs can be characterized as belonging to successive generations. First-generation CARs typically include a single-chain variable fragment of an antibody specific for an antigen, for example including a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD35 or scFv-FcRγ; see U.S. Pat. Nos. 7,741,465; 5,912,172; 5,906,936, each of which is specifically incorporated by reference herein in its entirety). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761, each of which is specifically incorporated by reference herein in its entirety). Third-generation CARs include a combination of costimulatory endodomains, such a CD3ζ-chain, CD97, GDI 1a-CD18, CD2, ICOS, CD27, CD154, CDS, OX40, 4-1BB, or CD28 signaling domains (for example scFv-CD28-4-1BB-CD3ζ or scFv-CD28-OX40-CD32; see U.S. Pat. Nos. 8,906,682; 8,399,645; 5,686,281; PCT Publication No. WO2014134165; PCT Publication No. WO2012079000, each of which is specifically incorporated by reference herein in its entirety). Alternatively, co-stimulation can be orchestrated by expressing CARs in antigen-specific T cells, chosen so as to be activated and expanded following engagement of their native αβTCR, for example by antigen on professional antigen-presenting cells, with attendant co-stimulation. Any of the first, second, or third generation CARs described above can be used in accordance with the disclosed compositions and methods.

E. Nucleic Acids Encoding anti-LANCL1 Antibodies and Antigen-Binding Fragments

Isolated nucleic acids and vectors encoding or expressing anti-LANCL1 monoclonal antibodies are also provided. As used herein, “isolated nucleic acid” refers to a nucleic acid that is separated from other nucleic acid molecules that are present in a mammalian genome, including nucleic acids that normally flank one or both sides of the nucleic acid in a mammalian genome.

An isolated nucleic acid can be, for example, a DNA molecule, provided one of the nucleic acid sequences normally found immediately flanking that DNA molecule in a naturally occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be single strand or double stranded, and can be in sense or antisense orientation, or can be complementary to a reference sequence. Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

### 1. Nucleic Acid Sequences Encoding the VH

A nucleic acid sequence encoding the heavy chain variable region (VH) of the anti-LANCL1 monoclonal antibody is:

(SEQ ID NO: 38)
ATGGGATGGAGCTGGATCTTTCTCTTTCTCCTGTCAGGAACTGCAGGTGTCCTCTCTGA
GGTCCAGTTGCAACAATCTGGACCTGAGCTGGTGAAGCCTGGGGCTTCAGTGAAGATAT
CCTGTAAGGCTTCTGGATACACGTTCACTGACTACTACATGAACTGGGTGAAGCAGAGC
CATGGAAAGAGCCTTGAGTGGATTGGAGATATTAATCCTAACAATGGTGGTGCTAGCTA
CAACCAGAAGTTCAAGGGCAAGGCCACATTGACTGTAGACAAGTCCTCCAGCACAGCCT
ACATGGAGCTCCGCAGCCTGACATCTGAGGACTCTGCAGTCTATTACTGTGTAAGATCG
GGCGATGGTTACTACTTTGCTTCCTGGGGCCAAGGGACTCTGCTCACTGTCTCTGCAGC
CAAAACAACACCCCCATCAGTCTATCCACTGGCCCCTGGGTGTGGAGATACAACTGGTT
CCTCTGTGACTCTGGGATGCCTGGTCAAGGGCTACTTCCCTGAGTCAGTGACTGTGACT
TGGAACTCTGGATCCCTGTCCAGCAGTGTGCACACCTTCCCAGCTCTCCTGCAGTCTGG
ACTCTACACTATGAGCAGCTCAGTGACTGTCCCCTCCAGCACCTGGCCAAGTCAGACCG
TCACCTGCAGCGTTGCTCACCCAGCCAGCAGCACCACGGTGGACAAAAAACTTGAGCCC
AGCGGGCCCATTTCAACAATCAACCCCTGTCCTCCATGCAAGGAGTGTCACAAATGCCC
AGCTCCTAACCTCGAGGGTGGACCATCCGTCTTCATCTTCCCTCCAAATATCAAGGATG
TACTCATGATCTCCCTGACACCCAAGGTCACGTGTGTGGTGGTGGATGTGAGCGAGGAT
GACCCAGACGTCCGGATCAGCTGGTTTGTGAACAACGTGGAAGTACACACAGCTCAGAC
ACAAACCCATAGAGAGGATTACAACAGTACTATCCGGGTGGTCAGTGCCCTCCCCATCC
AGCACCAGGACTGGATGAGTGGCAAGGAGTTCAAATGCAAGGTCAACAACAAAGACCTC
CCATCACCCATCGAGAGAACCATCTCAAAAATTAAAGGGCTAGTCAGAGCTCCACAAGT
ATACATCTTGCCGCCACCAGCAGAGCAGTTGTCCAGGAAAGATGTCAGTCTCACTTGCC
TGGTCGTGGGCTTCAACCCTGGAGACATCAGTGTGGAGTGGACCAGCAATGGGCATACA
GAGGAGAACTACAAGGACACCGCACCAGTCCTGGACTCTGACGGTTCTTACTTCATATA
CAGCAAGCTCGATATAAAAACAAGCAAGTGGGAGAAAACAGATTCCTTCTCATGCAACG
TGAGACACGAGGGTCTGAAAAATTACTACCTGAAGAAGACCATCTCCCGGTCTCCGGGT
AAATGA. TGA represents the stop codon.

A second nucleic acid sequence encoding the VH of the anti-LANCL1 monoclonal antibody is:

(SEQ ID NO: 39)
ATGGGATGGAGCTGGATCTTTCTCTTCCTCCTGTCAGGAACTGCAGGTGTCCACTCTGA
GGTCCAGCTGCAACAGTCTGGACCTGTGCTGGTGAAGCCTGGGGCTTCAGTGAAGATGT
CCTGTAAGGCTTCTGGATACACATTCACTGACTACTATATGAACTGGGTGAAGCAGAGC
CATGGAAAGAGCCTTGAGTGGATTGGAGTTATTAATCCTTACAACGGTCATACTAACTA
CAACCAGAAGTTCAAGGGCAAGGCCACATTGACTGTTGACAAGTCCTCCAACACAGCCT
ACATGGAGCTCAACAGTCTGACATCTGAGGACTCTGCAGTCTATTACTGTGCAAGATTC
CCTTACTACGGTAGTAGCTATAGGGTTGACTACTGGGGCCAAGGCACCACTCTCACAGT
CTCCTCAGCCAAAACAACACCCCCATCAGTCTATCCACTGGCCCCTGGGTGTGGAGATA
CAACTGGTTCCTCTGTGACTCTGGGATGCCTGGTCAAGGGCTACTTCCCTGAGTCAGTG
ACTGTGACTTGGAACTCTGGATCCCTGTCCAGCAGTGTGCACACCTTCCCAGCTCTCCT
GCAGTCTGGACTCTACACTATGAGCAGCTCAGTGACTGTCCCCTCCAGCACCTGGCCAA
GTCAGACCGTCACCTGCAGCGTTGCTCACCCAGCCAGCAGCACCACGGTGGACAAAAAA
CTTGAGCCCAGCGGGCCCATTTCAACAATCAACCCCTGTCCTCCATGCAAGGAGTGTCA
CAAATGCCCAGCTCCTAACCTCGAGGGTGGACCATCCGTCTTCATCTTCCCTCCAAATA
TCAAGGATGTACTCATGATCTCCCTGACACCCAAGGTCACGTGTGTGGTGGTGGATGTG
AGCGAGGATGACCCAGACGTCCGGATCAGCTGGTTTGTGAACAACGTGGAAGTACACAC
AGCTCAGACACAAACCCATAGAGAGGATTACAACAGTACTATCCGGGTGGTCAGTGCCC
TCCCCATCCAGCACCAGGACTGGATGAGTGGCAAGGAGTTCAAATGCAAGGTCAACAAC
AAAGACCTCCCATCACCCATCGAGAGAACCATCTCAAAAATTAAAGGGCTAGTCAGAGC
TCCACAAGTATACATCTTGCCGCCACCAGCAGAGCAGTTGTCCAGGAAAGATGTCAGTC
TCACTTGCCTGGTCGTGGGCTTCAACCCTGGAGACATCAGTGTGGAGTGGACCAGCAAT
GGGCATACAGAGGAGAACTACAAGGACACCGCACCAGTCCTGGACTCTGACGGTTCTTA
CTTCATATACAGCAAGCTCGATATAAAAACAAGCAAGTGGGAGAAAACAGATTCCTTCT
CATGCAACGTGAGACACGAGGGTCTGAAAAATTACTACCTGAAGAAGACCATCTCCCGG
TCTCCGGGTAAATGA. TGA represents the stop codon.

The complementarity determining regions (CDRs) are shown in bold italic in SEQ ID NO:38 and SEQ ID NO:39, including a first, second, and third CDR regions, CDR-1, CDR-2, and CDR-3 respectively. Exemplary nucleic acid sequences for CDR-1 of the heavy chain variable region include GACTACTACATGAAC (SEQ ID NO:40) and GACTACTATATGAAC (SEQ ID NO:41). Exemplary nucleic acid sequences for CDR-2 of the heavy chain variable region include GATATTAATCCTAACAATGGTGGTGCTAGCTACAACCAGAAGTTCAAGGGC (SEQ ID NO:42), and GTTATTAATCCTTACAACGGTCATACTAACTACAACCAGAAGTTCAAGGGC (SEQ ID NO:43). Exemplary nucleic acid sequences for CDR-3 of the heavy chain variable region include TCGGGCGATGGTTACTACTTTGCTTCC (SEQ ID NO:44) and TTCCCTTACTACGGTAGTAGCTATAGGGTTGACTAC (SEQ ID NO:45). In some forms, the first, second, and third CDR regions of the heavy chain variable region include variant nucleic acid sequences having about 70%, about 80%, about 90%, about 95%, about 96%, about 97%, about 98%, about 99% sequence identity to SEQ ID NO:40, SEQ ID NO:41, SEQ ID NO:42, SEQ ID NO:43, SEQ ID NO:44 or SEQ ID NO:45.

The framework region (FR) regions are shown in underlining in SEQ ID NO:38 and SEQ ID NO:39, including a first, second, third, and fourth FR regions, FR1, FR2, FR3, and FR4 respectively. Exemplary nucleic acid sequences for FR1 of the heavy chain variable region include GAGGTCCAGTTGCAACAATCTGGACCTGAGCTGGTGAAGCCTGGGGCTTCAGTGAAGAT ATCCTGTAAGGCTTCTGGATACACGTTCACT (SEQ ID NO:46) and GAGGTCCAGCTGCAACAGTCTGGACCTGTGCTGGTGAAGCCTGGGGCTTCAGTGAAGAT GTCCTGTAAGGCTTCTGGATACACATTCACT (SEQ ID NO:47). An exemplary sequence for FR2 of the heavy chain variable region is TGGGTGAAGCAGAGCCATGGAAAGAGCCTTGAGTGGATTGGA (SEQ ID NO:48). Exemplary nucleic acid sequences for FR3 of the heavy chain variable region include AAGGCCACATTGACTGTAGACAAGTCCTCCAGCACAGCCTACATGGAGCTCCGCAGCCT GACATCTGAGGACTCTGCAGTCTATTACTGTGTAAGA (SEQ ID NO:49) and AAGGCCACATTGACTGTTGACAAGTCCTCCAACACAGCCTACATGGAGCTCAACAGTCT GACATCTGAGGACTCTGCAGTCTATTACTGTGCAAGA (SEQ ID NO:50). Exemplary sequences for FR4 of the heavy chain variable region include TGGGGCCAAGGGACTCTGCTCACTGTCTCTGCA (SEQ ID NO:51) and TGGGGCCAAGGCACCACTCTCACAGTCTCCTCA (SEQ ID NO:52). In some forms, the first, second, third, and fourth FR regions of the heavy chain variable region include variant nucleic acid sequences having about 70%, about 80%, about 90%, about 95%, about 96%, about 97%, about 98%, about 99% sequence identity to SEQ ID NO:46, SEQ ID NO:47, SEQ ID NO:48, SEQ ID NO:49, SEQ ID NO:50, SEQ ID NO:51, and/or SEQ ID NO:52.

The nucleic acid sequences encoding the VH signal peptides are shown in italic in SEQ ID NO:38 and SEQ ID NO:39. Exemplary nucleic acid sequences encoding the VH signal peptide include ATGGGATGGAGCTGGATCTTTCTCTTTCTCCTGTCAGGAACTGCAGGTGTCCTCTCT (SEQ ID NO:53) and ATGGGATGGAGCTGGATCTTTCTCTTCCTCCTGTCAGGAACTGCAGGTGTCCACTCT (SEQ ID NO:54). In some forms, the nucleic acid sequence encoding the VH signal peptide includes variant nucleic acid sequences having about 70%, about 80%, about 90%, about 95%, about 96%, about 97%, about 98%, about 99% sequence identity to SEQ ID NO:53 or SEQ ID NO:54.

### 2. Nucleic Acids Encoding the VL

An nucleic acid sequence encoding the light chain variable region (VL) of the anti-LANCL1 monoclonal antibody is:

(SEQ ID NO: 55)
ATGGGTACTGCTGCTCTGGGTTCCAGGTTCCACTGGTGACATTGTGCTGACACAGTCTC
CTGCTTCCTTAGCTGTATCTCTGGGGCAGAGGGCCACCATCTCATACAGGGCCAGCAAA
AGTGTCAGTACATCTGGCTATAGTTATATGCACTGGAACCAACAGAAACCAGGACAGCC
ACCCAGACTCCTCATCTATCTTGTATCCAACCTAGAATCTGGGGTCCCTGCCAGGTTCA
GTGGCAGTGGGTCTGGGACAGACTTCACCCTCAACATCCATCCTGTGGAGGAGGAGGAT
GCTGCAACCTATTACTGTCAGCACATTAGGGAGCTTACACGTTCGGAGGGGGGACCAAG
CTGGAAATAAAACGGGCTGATGCTGCACCAACTGTATCCATCTTCCCACCATCCAGTGA
GCAGTTAACATCTGGAGGTGCCTCAGTCGTGTGCTTCTTGAACAACTTCTACCCCAAAG
ACATCAATGTCAAGTGGAAGATTGATGGCAGTGAACGACAAAATGGCGTCCTGAACAGT
TGGACTGATCAGGACAGCAAAGACAGCACCTACAGCATGAGCAGCACCCTCACGTTGAC
CAAGGACGAGTATGAACGACATAACAGCTATACCTGTGAGGCCACTCACAAGACATCAA
CTTCACCCATTGTCAAGAGCTTCAACAGGAATGAGTGTTAG. TAG
represents the stop codon.

A second nucleic acid sequence encoding light chain variable region (VL) of the anti-LANCL1 monoclonal antibody is:

(SEQ ID NO: 56)
ATGGTTTTCACACCTCAGATACTTGGACTTATGCTTTTTTGGATTTCAGCCTCCAGAGG
TGATATTGTGCTAACTCAGTCTCCAGCCACCCTGTCTGTGACTCCAGGAGATAGCGTCA
GTCTTTCCTGCAGGGCCAGCCAAAGTATTAGCAACAACCTACACTGGTATCAACAAAAA
TCACATGAGTCTCCAAGGCTTCTCATCAAGTATGCTTCCCAGTCCATCTCTGGGATCCC
CTCCAGGTTCAGTGGCAGTGGATCAGGGACAGATTTCACTCTCAGTATCAACAGTGTGG
AGACTGAAGATTTTGGAATGTATTTCTGTCAACAAATTAACAGCTGGCCTCTCACGTTC
GGTGCTGGGACCAAGCTGGAGCTGAAACGGGCTGATGCTGCACCAACTGTATCCATCTT
CCCACCATCCAGTGAGCAGTTAACATCTGGAGGTGCCTCAGTCGTGTGCTTCTTGAACA
ACTTCTACCCCAAAGACATCAATGTCAAGTGGAAGATTGATGGCAGTGAACGACAAAAT
GGCGTCCTGAACAGTTGGACTGATCAGGACAGCAAAGACAGCACCTACAGCATGAGCAG
CACCCTCACGTTGACCAAGGACGAGTATGAACGACATAACAGCTATACCTGTGAGGCCA
CTCACAAGACATCAACTTCACCCATTGTCAAGAGCTTCAACAGGAATGAGTGTTAG.
TAG represents the stop codon.

The complementarity determining regions (CDRs) are shown in bold italic in SEQ ID NO:55 and SEQ ID NO:56, including a first, second, and third CDR regions, CDR-1, CDR-2, and CDR-3 respectively. Exemplary nucleic acid sequences for CDR-1 of the light chain variable region include: AGGGCCAGCAAAAGTGTCAGTACATCTGGCTATAGTTATATGCAC (SEQ ID NO:57) and AGGGCCAGCCAAAGTATTAGCAACAACCTACA (SEQ ID NO:58). Exemplary nucleic acid sequences for CDR-2 of the light chain variable region include CTTGTATCCAACCTAGAATCT (SEQ ID NO:59), and TATGCTTCCCAGTCCATCTCT (SEQ ID NO:60). Exemplary nucleic acid sequences for CDR-3 of the light chain variable region include CAGCACATTAGGGAGCTTACACG (SEQ ID NO:61) and CAACAAATTAACAGCTGGCCTCTCACG (SEQ ID NO:62). In some forms, the first, second, and third CDR regions of the light chain variable region include variant nucleic acid sequences having about 70%, about 80%, about 90%, about 95%, about 96%, about 97%, about 98%, about 99% sequence identity to SEQ ID NOs: 57-62.

The framework region (FR) regions are shown in underlining in SEQ ID NO:55 and SEQ ID NO:56, including a first, second, third, and fourth FR regions, FR1, FR2, FR3, and FR4 respectively. Exemplary nucleic acid sequences for FR1 of the light chain variable region include GACATTGTGCTGACACAGTCTCCTGCTTCCTTAGCTGTATCTCTGGGGCAGAGGGCCAC CATCTCATAC (SEQ ID NO:63) and GATATTGTGCTAACTCAGTCTCCAGCCACCCTGTCTGTGACTCCAGGAGATAGCGTCAG TCTTTCCTGC (SEQ ID NO:64). An exemplary sequence for FR2 of the light chain variable region is TGGAACCAACAGAAACCAGGACAGCCACCCAGACTCCTCATCTAT (SEQ ID NO:65) and CTGGTATCAACAAAAATCACATGAGTCTCCAAGGCTTCTCATCAAG (SEQ ID NO:66). Exemplary nucleic acid sequences for FR3 of the light chain variable region include GGGGTCCCTGCCAGGTTCAGTGGCAGTGGGTCTGGGACAGACTTCACCCTCAACATCCA TCCTGTGGAGGAGGAGGATGCTGCAACCTATTACTGT (SEQ ID NO:67) and GGGATCCCCTCCAGGTTCAGTGGCAGTGGATCAGGGACAGATTTCACTCTCAGTATCAA CAGTGTGGAGACTGAAGATTTTGGAATGTATTTCTGT (SEQ ID NO:68). Exemplary sequences for FR4 of the light chain variable region include TTCGGAGGGGGGACCAAGCTGGAAATAAAA (SEQ ID NO:69) and TTCGGTGCTGGGACCAAGCTGGAGCTGAAA (SEQ ID NO:70). In some forms, the first, second, third, and fourth FR regions of the light chain variable region include variant nucleic acid sequences having about 70%, about 80%, about 90%, about 95%, about 96%, about 97%, about 98%, about 99% sequence identity to SEQ ID NOS: 63-70.

The nucleic acid sequences encoding the VL signal peptides are shown in italic in SEQ ID NO:55 and SEQ ID NO:56. Exemplary nucleic acid sequences encoding the VH signal peptide include ATGGGTACTGCTGCTCTGGGTTCCAGGTTCCACTGGT (SEQ ID NO:71) and ATGGTTTTCACACCTCAGATACTTGGACTTATGCTTTTTTGGATTTCAGCCTCCAGAGG T (SEQ ID NO:72). In some forms, the nucleic acid sequence encoding the VL signal peptide includes variant nucleic acid sequences having about 70%, about 80%, about 90%, about 95%, about 96%, about 97%, about 98%, about 99% sequence identity to SEQ ID NO:71 or SEQ ID NO:72.

Nucleic acids, such as those described above, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, CT), maltose E binding protein and protein A. In one embodiment, the variant PD-L2 fusion protein is present in a vector containing nucleic acids that encode one or more domains of an Ig heavy chain constant region, preferably having an amino acid sequence corresponding to the hinge, C_{H2} and C_{H3} regions of a human immunoglobulin Cγ1 chain.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the anti-LANCL1 polypeptides described herein.

### F. Formulations

The disclosed compositions can be formulated as part of a pharmaceutical formulation for administration to a subject in vivo. The disclosed compositions containing the anti-LANCL1 antibodies and/or antigen-binding fragments can be formulated as part of a pharmaceutical formulation for administration to a subject in need thereof. Typically, the pharmaceutical formulation contains the anti-LANCL1 antibodies or antigen-binding fragments thereof, a pharmaceutically acceptable excipient, and/or a pharmaceutically acceptable carrier suitable for administration into the body of a subject e.g., human patient.

In some forms, the disclosed formulations can used in a mono or combination therapy. The combination therapies can include administration of an effective amount of the anti-LANCL1 antibodies together in the same admixture, or in separate formulations. In some embodiments, the pharmaceutical formulations can include one or more additional active agents. Therefore, in some embodiments, the pharmaceutical formulations include two, three, or more active agents.

The pharmaceutical formulations can be formulated as a pharmaceutical dosage unit, referred to as a unit dosage form. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rats, rabbits, dogs, or pigs. The animal model is also used to achieve a desirable concentration range and route of administration. Such information should then be useful to determine useful doses and routes for administration in humans.

### 1. Delivery Vehicles

Appropriate delivery vehicles for the disclosed anti-LANCL1 antibodies are known in the art. For example, in some embodiments, the active agent(s) is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof.

### 2. Exemplary Formulations

Pharmaceutical compositions can be for administration by parenteral (intramuscular, intraperitoneal, intravenous (IV) or subcutaneous injection), enteral, or transmucosal routes of administration or using bio erodible inserts and can be formulated in dosage forms appropriate for each route of administration.

In certain embodiments, the compositions are administered locally, for example, by injection directly into a site to be treated (e.g., into a tumor). In some embodiments, the compositions are injected or otherwise administered directly into the vasculature onto vascular tissue at or adjacent to the intended site of treatment (e.g., adjacent to a tumor). Typically, local administration causes an increased localized concentration of the compositions which is greater than that which can be achieved by systemic administration. Targeting of the molecules or formulation can be used to achieve more selective delivery.

a. Formulations for Parenteral Administration

The anti-LANCL1 antibodies and antigen-binding fragments can be formulated for parenteral administration. For example, parenteral administration may include administration to a patient intravenously, intradermally, intraperitoneally, intramuscularly, subcutaneously, by injection, by infusion, etc.

In some forms, the anti-LANCL1 antibodies and/or antigen-binding fragments can be administered in an aqueous solution, by parenteral injection. The formulation may also be in the form of a suspension or emulsion. In general, pharmaceutical compositions include effective amounts of the anti-LANCL1 antibodies and/or antigen-binding fragments and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), antioxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacterium retaining filter, by incorporating sterilizing agents into the compositions, by irradiating the compositions, or by heating the compositions.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

The parenteral formulations described herein can be formulated for controlled release including immediate release, delayed release, extended release, pulsatile release, and combinations thereof.

b. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants. Delayed release dosage formulations may be prepared as described in standard references. These references provide information on carriers, materials, equipment, and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone® XL from GAF Chemical Corp).

Stabilizers are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Oral dosage forms, such as capsules, tablets, solutions, and suspensions, can be formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation into the finished dosage form.

In another embodiment, one or more compounds and optionally, one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more antibodies or antigen binding fragments thereof, and optional one or more additional active agents are formulated into a sold oral dosage form, such as a tablet or capsule, and the solid dosage form is coated with one or more controlled release coatings, such as a delayed release coatings or extended-release coatings. The coating or coatings may also contain antibodies or antigen binding fragments thereof and/or additional active agents.

The extended-release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and ® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

Alternatively, extended-release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form including single or multiple units. Examples of multiple units include, but are not limited to, multilayer tablets and capsules containing tablets, beads, or granules. An immediate release portion can be added to the extended-release system by means of either applying an immediate release layer on top of the extended-release core using a coating or compression process or in a multiple unit system such as a capsule containing extended and immediate release beads.

Extended-release tablets containing hydrophilic polymers are prepared by techniques commonly known in the art, such as direct compression, wet granulation, or dry granulation processes. Their formulations usually incorporate polymers, diluents, binders, and lubricants as well as the active pharmaceutical ingredients. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bio erodible, gradually hydrolysable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxy methylcellulose, hydroxypropyl methylcellulose, hydroxypropyl methylcellulose acetate succinate, hydroxypropyl methylcellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinyl acetate phthalate, vinyl acetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 weight % to 100 weight % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e., non-aqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the composition. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

### III. Methods of Use

Methods of treatment are also provided and can be used alone or in combination with other methods disclosed herein such as the disclosed methods of detection, diagnosis, prognosis, and treatment monitoring. The methods typically include administering a subject in need thereof an effective amount of a disclosed composition, e.g., a humanized anti-LANCL1 monoclonal antibody, to treat the subject. This is particularly true where disease or condition is characterized by increased expression or presence of LANCL1 protein.

### A. Effective Amounts

The effective amount or therapeutically effective amount of a pharmaceutical compositions including for example anti-LANCL1 antibodies, anti-LANCL1 antigen-binding fragments or cells thereof, such as therapeutic T cells, can be a dosage sufficient to treat, inhibit, or alleviate one or more symptoms of a disease or disorder, such as hepatocellular cancer, or to otherwise provide a desired pharmacologic and/or physiologic effect, for example, reducing, inhibiting, or reversing one or more of the underlying pathophysiological mechanisms underlying a disease or disorder, such as cancer or autoimmune disease Any liver cancer that is LANCL1-positive can be treated using the disclosed compositions and formulations thereof. Exemplary cancers that can be treated include but are not limited to fibrolamellar hepatocellular carcinoma, bile duct cancer and cholangiocarcinoma.

In some forms, when administrating the pharmaceutical composition, the amount administered can be expressed as the amount effective to achieve a desired anti-cancer effect in the recipient. For example, in some forms, the amount of the pharmaceutical composition is effective to inhibit the viability or proliferation of hepatocellular cancer cells in the recipient. In some forms, the amount of the pharmaceutical composition including anti-LANCL1 antibodies and modified cells thereof, such as therapeutic T cells, is effective to reduce the tumor burden in the recipient, or reduce the total number of cancer cells, and combinations thereof. In other forms, the amount of the pharmaceutical compositions including anti-LANCL1 antibodies is effective to reduce one or more symptoms or signs of HCC in a cancer patient, or signs of liver tumors in a patient having liver cancer.

The effective amount of the pharmaceutical composition will vary based on the active agent and from subject to subject, depending on the species, age, weight and general condition of the subject, the severity of the disorder being treated, and its mode of administration. Thus, it is not possible to specify an exact amount for every pharmaceutical composition. However, an appropriate amount can be determined by one of ordinary skill in the art using only routine experimentation given the teachings herein. For example, effective dosages and schedules for administering the pharmaceutical composition can be determined empirically. In some forms, the dosage ranges for the administration of the composition are those large enough to effect reduction in cancer cell proliferation or viability, or to reduce tumor burden for example.

Preferably, the dosage is not so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the composition can increase or decrease over the course of a particular treatment. Changes in dosage can result and become apparent from the results of diagnostic assays.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

For example, some embodiments, antibodies are packaged in a hermetically sealed container, such as an ampoule or sachets, indicating the quantity of antibody. In some embodiments, the antibodies are supplied as a dry sterilized lyophilized powder or water free concentrate in a hermetically sealed container and can be reconstituted, e.g., with water or saline to the appropriate concentration for administration to a subject. Preferably, the antibodies of the invention are supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, or at least 75 mg. The lyophilized antibodies can be stored at between 2 and 8° C. in their original container and the antibodies can be administered within 12 hours, preferably within 6 hours, within 5 hours, within 3 hours, or within 1 hour after being reconstituted. In an alternative embodiment, antibodies can be supplied in liquid form in a hermetically sealed container indicating the quantity and concentration of the antibody, fusion protein, or conjugated molecule. Preferably, the liquid form of the antibodies is supplied in a hermetically sealed container at least 1 mg/ml, more preferably at least 2.5 mg/ml, at least 5 mg/ml, at least 8 mg/ml, at least 10 mg/ml, at least 15 mg/ml, at least 25 mg/ml, at least 50 mg/ml, at least 100 mg/ml, at least 150 mg/ml, at least 200 mg/ml of the antibodies.

In some forms, the dosage of the anti-LANCL1 antibodies administered to a patient range from about 0.01 mg/kg to 100 mg/kg of the patient's body weight. Preferably, the dosage administered to a patient is between 0.01 mg/kg and 20 mg/kg, 0.01 mg/kg and 10 mg/kg, 0.01 mg/kg and 5 mg/kg, 0.01 and 2 mg/kg, 0.01 and 1 mg/kg, 0.01 mg/kg and 0.75 mg/kg, 0.01 mg/kg and 0.5 mg/kg, 0.01 mg/kg to 0.25 mg/kg, 0.01 to 0.15 mg/kg, 0.01 to 0.10 mg/kg, 0.01 to 0.05 mg/kg, or 0.01 to 0.025 mg/kg of the patient's body weight. In particular, the invention contemplates that the dosage administered to a patient is 0.2 mg/kg, 0.3 mg/kg, 1 mg/kg, 3 mg/kg, 6 mg/kg or 10 mg/kg. A dose as low as 0.01 mg/kg may show appreciable pharmacodynamic effects. Dose levels of 0.10-1 mg/kg are predicted to be most appropriate. Higher doses (e.g., 1 mg/kg to 60 mg/kg) are also contemplated. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies may be reduced by enhancing uptake and tissue penetration of the antibodies by modifications such as, for example, lipidation.

It can generally be stated that a pharmaceutical composition containing CAR cells described herein can be administered at a dosage of 10⁴ to 10⁹ cells/kg body weight, preferably 10⁵ to 10⁷ cells/kg body weight, including all integer values within those ranges. In some forms, patients can be treated by infusing a disclosed pharmaceutical composition containing CAR expressing cells (e.g., T cells) in the range of about 10⁴ to 10¹² or more cells per square meter of body surface (cells/m).

Injections and infusion of the disclosed compositions can be repeated as often and as many times as the patient can tolerate until the desired response is achieved. Thus, antibodies and CAR cell compositions can also be administered once or multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can be determined by one skilled in the art of medicine by monitoring the patient for signs of disease and adjusting the treatment accordingly. In some forms, the unit dosage is in a unit dosage form for intravenous injection. In some forms, the unit dosage is in a unit dosage form for oral administration. In some forms, the unit dosage is in a unit dosage form for inhalation. In some forms, the unit dosage is in a unit dosage form for intra-tumoral injection.

Treatment can be continued for an amount of time sufficient to achieve one or more desired therapeutic goals, for example, a reduction of the amount of cancer cells relative to the start of treatment, or complete absence of cancer cells in the recipient. Treatment can be continued for a desired period of time, and the progression of treatment can be monitored using any means known for monitoring the progression of anti-cancer treatment in a patient. In some forms, administration is carried out every day of treatment, or every week, or every fraction of a week. In some forms, treatment regimens are carried out over the course of up to two, three, four or five days, weeks, or months, or for up to 6 months, or for more than 6 months, for example, up to one year, two years, three years, or up to five years.

The efficacy of administration of a particular dose of the pharmaceutical compositions including modified cells, such as therapeutic T cells, according to the methods described herein can be determined by evaluating the aspects of the medical history, signs, symptoms, and objective laboratory tests that are known to be useful in evaluating the status of a subject in need for the treatment of cancer or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious. In some forms, efficacy is assessed as a measure of the reduction in tumor volume and/or tumor mass at a specific time point (e.g., 1-5 days, weeks, or months) following treatment.

### B. Modes of Administration

Any of the disclosed compositions can be used therapeutically in combination with a pharmaceutically acceptable carrier. The compositions described herein can be conveniently formulated into pharmaceutical compositions composed of one or more of the compounds in association with a pharmaceutically acceptable carrier. See, e.g., Remington's Pharmaceutical Sciences, latest edition, by E. W. Martin Mack Pub. Co., Easton, PA, which discloses typical carriers and conventional methods of preparing pharmaceutical compositions that can be used in conjunction with the preparation of formulations of the therapeutics described herein and which is incorporated by reference herein. These most typically would be standard carriers for administration of compositions to humans. In one aspect, for humans and non-humans, these include solutions such as sterile water, saline, and buffered solutions at physiological pH. Other therapeutics can be administered according to standard procedures used by those skilled in the art.

The pharmaceutical compositions including antibodies or modified cells, such as therapeutic T cells, described herein can include, but are not limited to, carriers, thickeners, diluents, buffers, preservatives, surface active agents and the like in addition to the therapeutic(s) of choice.

Pharmaceutical compositions containing antibodies or modified cells, such as therapeutic T cells, and optionally one or more additional therapeutic agents can be administered to the subject in a number of ways depending on whether local or systemic treatment is desired, and on the area to be treated. Thus, for example, a pharmaceutical composition can be administered as an intravenous infusion, or directly injected into a specific site, for example, into or surrounding a tumor. Moreover, a pharmaceutical composition can be administered to a subject orally or parenterally, for example, by subcutaneous, intramuscular, intraperitoneal, intraarterial such as by hepatic arterial infusion or transarterial chemoembolization, intralymphatic, intravenous, intrathecal routes. In some forms, the compositions are administered directly into a tumor or tissue, e.g., stereotactically.

In some forms, a pharmaceutical composition containing LANCL1-specific antibodies can contact healthy cells of subject and/or tumor cells or cancerous cells. In certain embodiments, a LANCL1-specific antibody can contact cells in the liver, including cancerous liver cells. In certain embodiments, a humanized LANCL1-specific antibody can inhibit hepatocellular carcinoma and other solid tumors. The LANCL1-specific antibody can bind to protein on the surface of cells of a subject, including, for example, cancerous cells. In certain embodiments, the LANCL1-specific antibody can suppress the liver tumor initiating cell (LTIC) properties, namely and not limited to, in vitro sphere formation, in vivo tumorigenicity, chemoresistance to chemo-drugs including but not limited to cisplatin, 5-fluorouracil, etc., and expression of cancer stemness-related genes. In certain embodiments, the LANCL1-specific antibody can inhibit the growth of tumor cells. The LANCL1-specific antibody can inhibit the growth of tumor cells by modulating the LANCL1 expression and/or the function of LANCL1. In certain embodiments, the LANCL1-specific antibody can modify the intracellular ROS levels in cells, specifically in HCC cells. For example, cell surface LANCL1 can suppress intracellular ROS levels; therefore, a LANCL1-specific antibody can block such function, leading to an increase in intracellular ROS levels that can be lethal or damaging to the HCC cells.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant including a porous, non-porous, or gelatinous material).

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Administration of the pharmaceutical compositions containing antibodies or genetically modified cells (e.g., CAR cells) can be localized (i.e., to a particular region, physiological system, tissue, organ, or cell type) or systemic.

### C. Combination Therapies

HCC can be treated curatively by local ablation, surgical resection, or liver transplantation. Treatment selection depends on tumor characteristics, the severity of underlying liver dysfunction, age, other medical comorbidities, and available medical resources and local expertise. Catheter-based locoregional treatment is used in patients with intermediate-stage cancer. Kinase and immune checkpoint inhibitors have been shown to be effective treatment options in patients with advanced-stage HCC.

The disclosed compositions can be administered to a subject in need thereof, alone or in combination with one or more adjunct therapies or procedures or can be an adjunct therapy to one or more primary therapies or producers. The additional therapy or procedure can be simultaneous or sequential with the combination therapy. In some forms, additional therapy is performed between drug cycles or during a drug holiday that is part of the combination therapy dosage regime. In preferred forms, the additional therapy is a conventional treatment for cancer, more preferably a conventional treatment for liver cancer. For example, in some forms, the additional therapy or procedure is surgery, transplant surgery, radiation therapy, or chemotherapy.

Exemplary additional therapeutic agents include conventional cancer therapeutics such as chemotherapeutic agents, cytokines, chemokines, and radiation therapy. Most chemotherapeutic drugs can be divided into alkylating agents, antimetabolites, anthracyclines, plant alkaloids, topoisomerase inhibitors, and other antitumor agents. These drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors). In a particular form, combination therapies used simultaneously or sequentially with a regime of a chemotherapeutic agent, e.g., Gemcitabine (Gemzar), Oxaliplatin (Eloxatin), Cisplatin, Doxorubicin (pegylated liposomal doxorubicin), Capecitabine (Xeloda), Mitoxantrone (Novantrone), docetaxel or cabazitaxel. In some forms, the adjunct or additional therapy is part of the combination therapy.

Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

Representative anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds including aflibercept (EYLEA®); MACUGEN® (pegaptanim sodium, anti-VEGF aptamer or EYE001) (Eyetech Pharmaceuticals); pigment epithelium derived factor(s) (PEDF); COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®); squalamine; endostatin; angiostatin; ribozyme inhibitors such as ANGIOZYME® (Sirna Therapeutics); multifunctional antiangiogenic agents such as NEOVASTAT® (AE-941) (Aeterna Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®), as well as other anti-angiogenesis agents known in the art.

In some forms, the compositions and methods are used prior to or in conjunction with surgical removal of tumors, for example, in preventing primary tumor metastasis. In some forms, the compositions and methods are used to enhance body's own anti-tumor immune functions. In some forms, the additional therapy or procedure can be simultaneous or sequential with the therapy or combination therapy. In some forms the additional therapy is performed between drug cycles or during a drug holiday that is part of a dosage regime. For example, in some forms, the additional therapy or procedure is surgery.

### IV. Kits

The compositions, reagents, and other materials for the disclosed compounds and cells can be packaged together in any suitable combination as a kit useful for performing, or aiding in the performance of, the methods. It is useful if the components in a given kit are designed and adapted for use together in the method. For example, kits with one or more compositions for administration to a subject, may include a pre-measured dosage of the composition in a sterile needle, ampule, tube, container, or other suitable vessel. The kits may include instructions for dosages and dosing regimens.

The kits can include printed instructions for administering the compound in a use as described above. The instructional material can include a publication, a recording, a diagram, or any other medium of expression which can be used to communicate the usefulness of the compositions and methods of the kit. Compositions can include antibodies in a solid (i.e., dry powder or lyophilized) form, or as a solution, such as an aqueous solution.

In one exemplary form, the kit contains:
(a) one or more single unit doses of a composition comprising the disclosed LANCL1 antibodies and/or antigen binding fragments thereof, pharmaceutical formulations thereof, and
(b) instructions on how the dose is to be administered for reducing one or more symptoms of HCC and/or reducing the proliferation of HCC in a subject e.g., a human patient.

The disclosed compositions and methods can be further understood through the following numbered paragraphs:

1. An antibody or antigen-binding fragment thereof, comprising a heavy chain variable region comprising three complementarity determining regions (CDRs) and a light chain variable region comprising three CDRs, wherein:
(i) the three heavy chain variable region CDRs comprise amino acid sequences selected from the group consisting of DYYMN (SEQ ID NO:5), DINPNNGGASYNQKFKG (SEQ ID NO:6), VINPYNGHTNYNQKFKG (SEQ ID NO:7), SGDGYYFAS (SEQ ID NO:8), FPYYGSSYRVDY (SEQ ID NO:9) respectively, or a functional variant thereof;
(ii) the three light chain variable region CDRs comprise amino acids sequences selected from the group consisting of RASKSVSTSGYSYMH (SEQ ID NO:21), RASQSISNNLH (SEQ ID NO:22), LVSNLES (SEQ ID NO:23), QHIRELT (SEQ ID NO:24), QQINSWPLT (SEQ ID NO:25), YASQSIS (SEQ ID NO:73), respectively, or a functional variant thereof; and
wherein the antibody or antigen binding fragment thereof binds to LanC Like Glutathione S-Transferase 1 (LANCL1) protein.
   2. The antibody or antigen-binding fragment thereof of paragraph 1, wherein:
(i) the three heavy chain variable region CDRs comprise the amino acids sequences DYYMN (SEQ ID NO:5), DINPNNGGASYNQKFKG (SEQ ID NO:6), SGDGYYFAS (SEQ ID NO:8), respectively, or DYYMN (SEQ ID NO:5), VINPYNGHTNYNQKFKG (SEQ ID NO:7), FPYYGSSYRVDY (SEQ ID NO:9) respectively, or a functional variant thereof; and
(ii) the three light chain variable region CDRs comprise the amino acids RASKSVSTSGYSYMH (SEQ ID NO:21), LVSNLES (SEQ ID NO:23), QHIRELT (SEQ ID NO:24) respectively, or RASQSISNNLH (SEQ ID NO:22), YASQSIS (SEQ ID NO:73), QQINSWPLT (SEQ ID NO:25), respectively, or a functional variant thereof;
   3. The antibody or antigen-binding fragment thereof of paragraphs 1 or 2, wherein:
(i) the three heavy chain variable region CDRs comprise the amino acid sequences: DYYMN (SEQ ID NO:5), DINPNNGGASYNQKFKG (SEQ ID NO:6), SGDGYYFAS (SEQ ID NO:8), respectively, or a functional variant thereof, and the three light chain variable region CDRs comprise the amino acid sequences: RASKSVSTSGYSYMH (SEQ ID NO:21), LVSNLES (SEQ ID NO:23), QHIRELT (SEQ ID NO:24) respectively, or a functional variant thereof; or
(ii) the three heavy chain variable region CDRs comprise the amino acid sequences: DYYMN (SEQ ID NO:5), VINPYNGHTNYNQKFKG (SEQ ID NO:7). FPYYGSSYRVDY (SEQ ID NO:9) respectively, or a functional variant thereof, and the three light chain variable region CDRs comprise amino acid sequences: RASQSISNNLH (SEQ ID NO:22), YASQSIS (SEQ ID NO:73), QQINSWPLT (SEQ ID NO:25), respectively, or a functional variant thereof.
   4. The antibody or antigen-binding fragment of any one of paragraphs 1-3, wherein the three heavy chain variable region CDRs comprise amino acids SEQ ID NO:5, SEQ ID NO:6, SEQ ID NO:8, respectively or a functional variant thereof, and the three light chain variable region CDRs comprise amino acids SEQ ID NO:21, SEQ ID NO:23, SEQ ID NO:24 respectively, or a functional variant thereof.
   5. The antibody or antigen-binding fragment of any one of paragraphs 1-3, wherein the three heavy chain variable region CDRs comprise amino acids SEQ ID NO:5, SEQ ID NO:7, SEQ ID NO:9 respectively, or a functional variant thereof, and the three light chain variable region CDRs comprise amino acids SEQ ID NO:22, SEQ ID NO:73, SEQ ID NO:25, respectively, or a functional variant thereof.
   6. The antibody or antigen-binding fragment of any one of paragraphs 1-3, wherein the three heavy chain variable region CDRs comprise amino acids SEQ ID NO:5, SEQ ID NO:7, SEQ ID NO:9 respectively, or a functional variant thereof, and the three light chain variable region CDRs comprise amino acids SEQ ID NO:21, SEQ ID NO:23, SEQ ID NO:24 respectively, or a functional variant thereof.
   7. The antibody or antigen-binding fragment of any one of paragraphs 1-3, wherein the three heavy chain variable region CDRs comprise amino acids SEQ ID NO:5, SEQ ID NO:6, SEQ ID NO:8 respectively, or a functional variant thereof, and the three light chain variable region CDRs comprise amino acids SEQ ID NO:22, SEQ ID NO:73, SEQ ID NO:25, respectively, or a functional variant thereof.
   8. The antibody or antigen-binding fragment of any one of paragraphs 1-7, wherein the functional variant has about 70%, about 75%, about 80%, about 85%, about 90%, about 95% or about 99% sequence identity to SEQ ID NO:5, SEQ ID NO:6, SEQ ID NO:8, SEQ ID NO:7, SEQ ID NO:9, SEQ ID NO:21, SEQ ID NO:23, SEQ ID NO:24, SEQ ID NO:22, SEQ ID NO:73 or SEQ ID NO:25.
   9. The antibody or antigen-binding fragment of any one of paragraphs 1-8, comprising a light chain variable region comprising the amino acid sequence of SEQ ID NO:19 and a heavy chain variable region comprising the amino acid sequence of SEQ ID NO:3.
   10. The antibody or antigen-binding fragment of any one of paragraphs 1-8, comprising a light chain variable region comprising the amino acid sequence of SEQ ID NO:20 and a heavy chain variable region comprising the amino acid sequence of SEQ ID NO:4.
   11. The antibody or antigen-binding fragment of any one of paragraphs 1-8, comprising a light chain variable region comprising the amino acid sequence of SEQ ID NO:19 and a heavy chain variable region comprising the amino acid sequence of SEQ ID NO:4.
   12. The antibody or antigen-binding fragment of any one of paragraphs 1-8, comprising a light chain variable region comprising the amino acid sequence of SEQ ID NO:20 and a heavy chain variable region comprising the amino acid sequence of SEQ ID NO:3.
   13. The antibody or antigen binding fragment thereof of any one of paragraphs 1-12, comprising one or more constant domains from an immunoglobulin constant region (Fc).
   14. The antibody or antigen binding fragment thereof of paragraph 13, wherein the constant domains are human constant domains selected from the group consisting of IgA, IgD, IgE, IgG, or IgM.
   15. The antibody or antigen binding fragment thereof of paragraph 14, wherein human IgG constant domain is selected from the group consisting of IgG1, IgG2, IgG3, or IgG4.
   16. The antibody or antigen-binding fragment of any one of paragraphs 1-15, wherein the antibody is a monoclonal antibody, a human antibody, a chimeric antibody, or a humanized antibody.
   17. A humanized antibody comprising one or more human IgG constant domains, a heavy chain variable region comprising three complementarity determining regions (CDRs) and a light chain variable region comprising three CDRs, wherein:
(i) the three heavy chain variable region CDRs comprising amino acid sequences selected from the group consisting of: DYYMN (SEQ ID NO:5), DINPNNGGASYNQKFKG (SEQ ID NO:6), VINPYNGHTNYNQKFKG (SEQ ID NO:7), SGDGYYFAS (SEQ ID NO:8), FPYYGSSYRVDY (SEQ ID NO:9) or a functional variant thereof;
(ii) the three light chain variable region CDRs comprise amino acid sequences selected from the group consisting of: RASKSVSTSGYSYMH (SEQ ID NO:21), RASQSISNNLH (SEQ ID NO:22), LVSNLES (SEQ ID NO:23), QHIRELT (SEQ ID NO:24), YASQSIS (SEQ ID NO:73), QQINSWPLT (SEQ ID NO:25), or a functional variant thereof; and
wherein the humanized antibody or antigen binding fragment thereof binds to human LanC Like Glutathione S-Transferase 1 (LANCL1) protein.
   18. The humanized antibody of paragraph 17, wherein:
(i) the three heavy chain variable region CDRs comprise amino acids DYYMN (SEQ ID NO:5), DINPNNGGASYNQKFKG (SEQ ID NO:6), SGDGYYFAS (SEQ ID NO:8), respectively, or DYYMN (SEQ ID NO:5), VINPYNGHTNYNQKFKG (SEQ ID NO:7), FPYYGSSYRVDY (SEQ ID NO:9) respectively, or a functional variant thereof; and
(ii) the three light chain variable region CDRs comprise amino acid sequences: RASKSVSTSGYSYMH (SEQ ID NO:21), LVSNLES (SEQ ID NO:23), QHIRELT (SEQ ID NO:24) respectively, or RASQSISNNLH (SEQ ID NO:22), YASQSIS (SEQ ID NO:73), QQINSWPLT (SEQ ID NO:25), respectively, or a functional variant thereof.
   9. The humanized antibody of paragraphs 17 or 18, wherein:
(i) the three heavy chain variable region CDRs comprise the amino acid sequences: DYYMN (SEQ ID NO:5), DINPNNGGASYNQKFKG (SEQ ID NO:6), SGDGYYFAS (SEQ ID NO:8), respectively, or a functional variant thereof, and the three light chain variable region CDRs comprise the amino acid sequences: RASKSVSTSGYSYMH (SEQ ID NO:21), LVSNLES (SEQ ID NO:23), QHIRELT (SEQ ID NO:24) respectively, or a functional variant thereof; or
(ii) the three heavy chain variable region CDRs comprise the amino acid sequences: DYYMN (SEQ ID NO:5), VINPYNGHTNYNQKFKG (SEQ ID NO:7), FPYYGSSYRVDY (SEQ ID NO:9) respectively, or a functional variant thereof, and the three light chain variable region CDRs comprise amino acid sequences: RASQSISNNLH (SEQ ID NO:22), YASQSIS (SEQ ID NO:73), QQINSWPLT (SEQ ID NO:25), respectively, or a functional variant thereof.
   20. The humanized antibody of any one of paragraphs 17-19, wherein the three heavy chain variable region CDRs comprise SEQ ID NO:5, SEQ ID NO:6, SEQ ID NO:8, respectively or a functional fragment thereof, and the three light chain variable region CDRs comprise SEQ ID NO:21, SEQ ID NO:23, SEQ ID NO:24 respectively, or a functional fragment thereof.
   21. The humanized antibody of any one of paragraphs 17-19, wherein the three heavy chain variable region CDRs comprise SEQ ID NO:5, SEQ ID NO:7, SEQ ID NO:9 respectively, or a functional fragment thereof, and the three light chain variable region CDRs comprise SEQ ID NO:22, SEQ ID NO:73, SEQ ID NO:25, respectively, or a functional fragment thereof.
   22. The humanized antibody of any one of paragraphs 17-19, wherein the three heavy chain variable region CDRs comprise SEQ ID NO:5, SEQ ID NO:6, SEQ ID NO:8 respectively, or a functional fragment thereof, and the three light chain variable region CDRs comprise SEQ ID NO:22, SEQ ID NO:73, SEQ ID NO:25, respectively, or a functional fragment thereof.
   23. The humanized antibody of any one of paragraphs 17-19, wherein the three heavy chain variable region CDRs comprise SEQ ID NO:5, SEQ ID NO:7, SEQ ID NO:9 respectively, or a functional fragment thereof, and the three light chain variable region CDRs comprise SEQ ID NO:21, SEQ ID NO:23, SEQ ID NO:24, respectively, or a functional fragment thereof.
   24. A nucleic acid encoding the antibody or antigen-binding fragment of any one of claims 1-16 or the humanized antibody of any one of paragraphs 17-23.
   25. A pharmaceutical composition comprising the antibody or antigen-binding fragment of any one of claims 1-16 or the humanized antibody of any one of paragraphs 17-23, and one or more pharmaceutically acceptable carriers and/or excipients.
   26. The pharmaceutical composition of paragraph 25, for use in a method of treating or preventing liver cancer in a subject.
   27. The pharmaceutical composition of paragraphs 25 or 26, wherein the subject has hepatocellular carcinoma.
   28. The pharmaceutical composition of any one of paragraphs 25-27, wherein the subject is at risk of developing hepatocellular carcinoma.
   29. A method of treating a subject in need thereof, comprising administering to the subject, an effective amount of the pharmaceutical composition of any one of paragraphs 25-28.
   30. The method of paragraph 29, wherein the subject has a disease or disorder caused by or characterized by increased presence of LanC Like Glutathione S-Transferase 1 (LANCL1) protein or a fragment thereof.
   31. The method of paragraphs 29 or 30, wherein the subject has liver cancer or is at risk of developing liver cancer.
   32. The method of any one of paragraphs 29-31, wherein the liver cancer is hepatocellular carcinoma.
   33. The method of any one of paragraphs 29-32, wherein the pharmaceutical composition is delivered via injection or infusion.
   34. The method of any one of paragraphs 29-33, wherein the pharmaceutical composition is effective to reduce the proliferation of tumor-initiating cells, sphere formation of hepatocellular carcinoma cells, and/or blocking or reducing the activity of LANCL1.
   35. The method of any one of paragraphs 29-34, wherein the administration is repeated every 4 hours, every 6 hours, every 12 hours, every day, optionally for a time period from 1 day to 1 month, from 1 day to 2 weeks, from 1 day to 1 week, or from 1 day to 3 days.
   36. The method of any one of paragraphs 29-35, further comprising administering one or more chemotherapeutic agent(s) to the subject.

### EXAMPLES

### Example 1. Generation and Screening of LANCL1 Monoclonal Antibodies

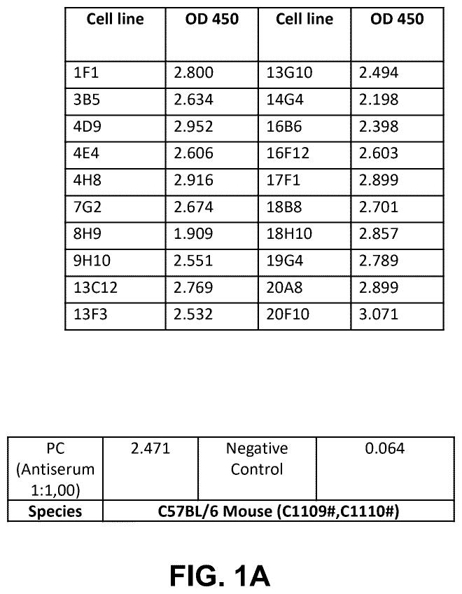

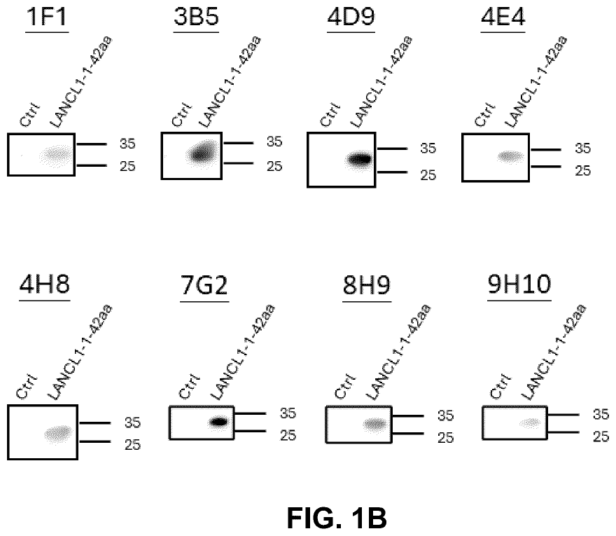

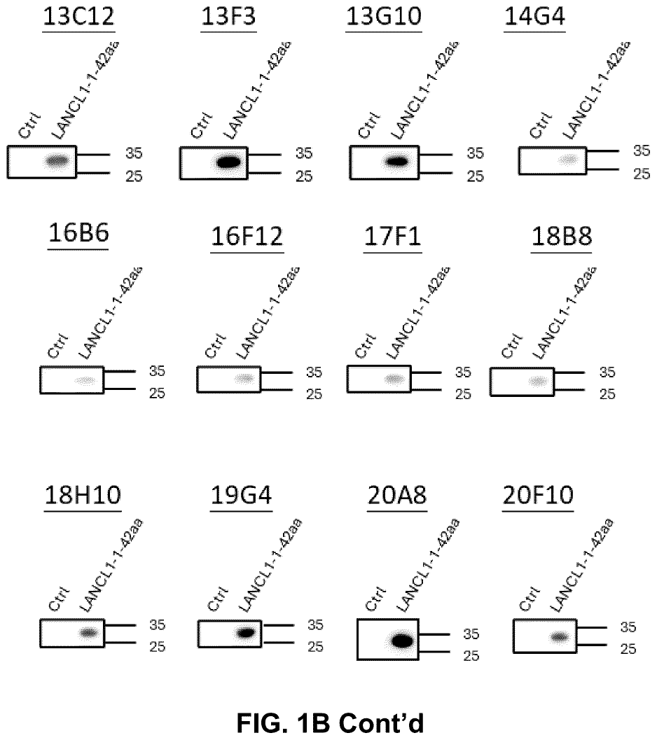

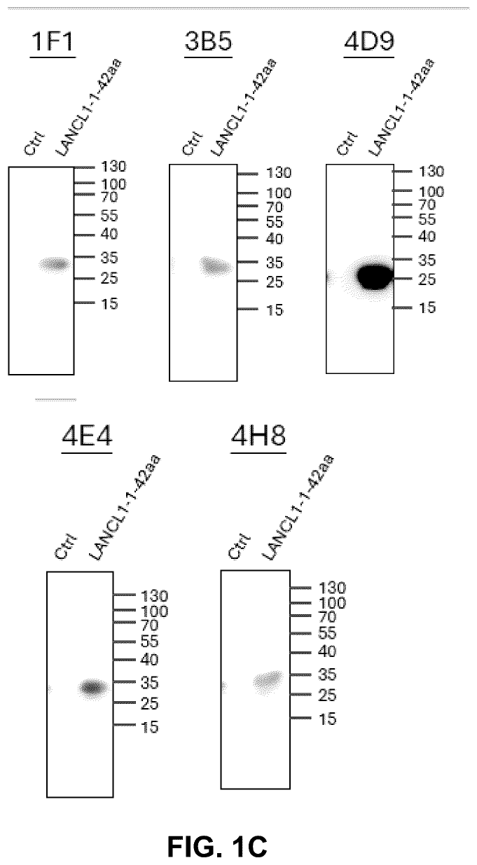

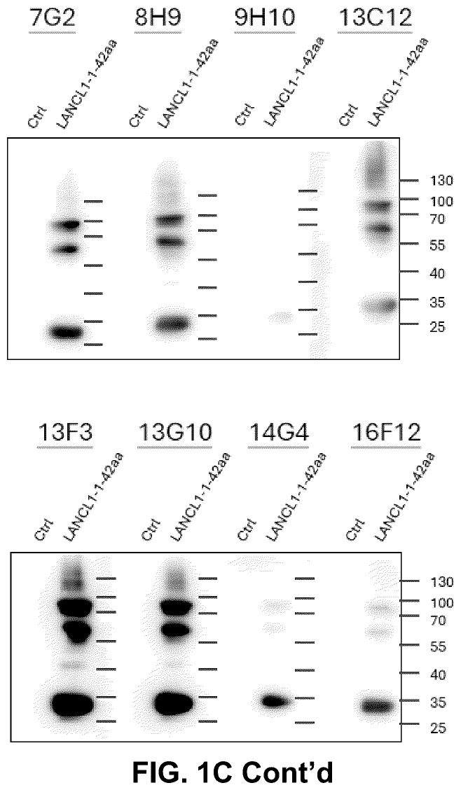

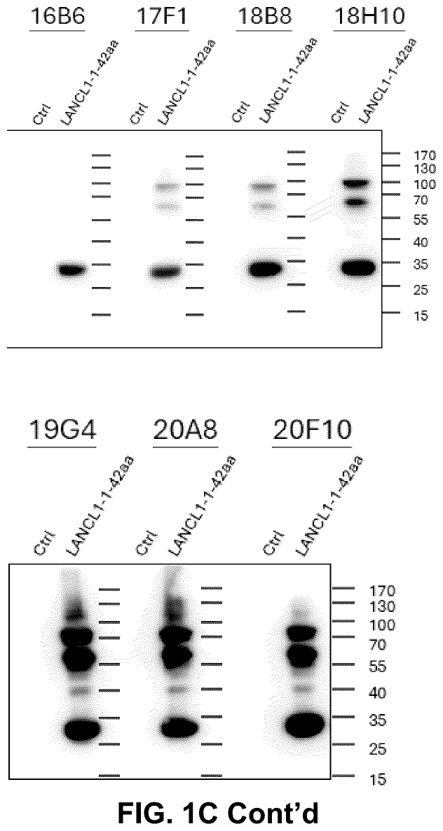

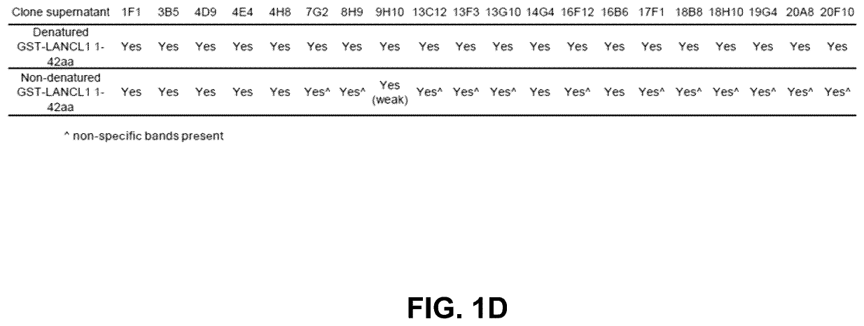

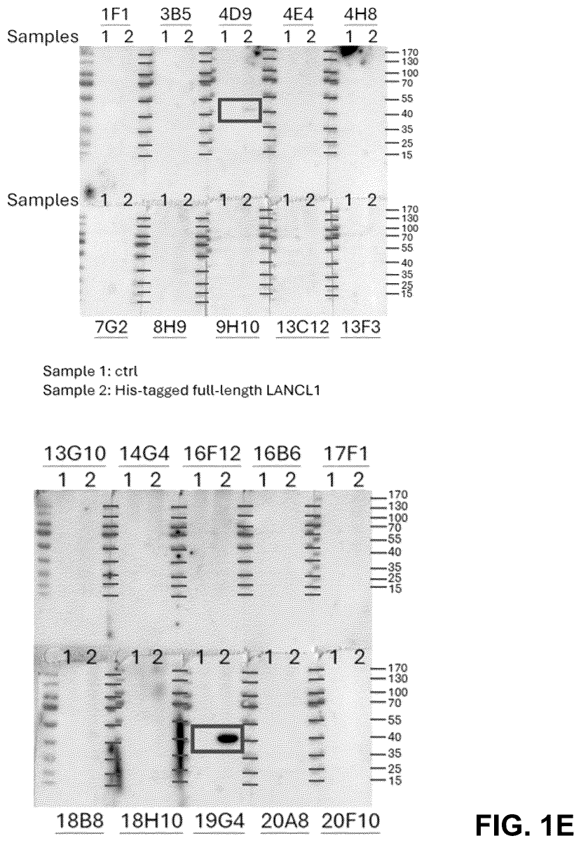

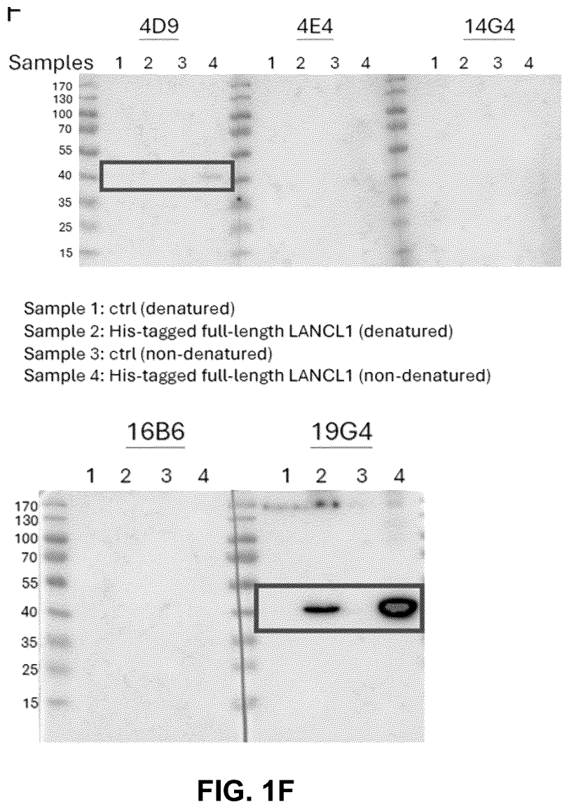

To generate specific antibodies targeting the N-terminal extracellular region of human LANCL1, the specific peptide corresponding to the N-terminal of human LANCL1 is used as immunogen. Since human LANCL1 and LANCL2 proteins share high homology in the middle region, the 1-42aa region which is specific to the LANCL1 protein was focused on. KLH was conjugated via a cysteine residue at the C terminal of the 1-42 aa peptide (MAQRAFPNPYADYNKSLAEGYFDAAGRLTPEFSQRL-TNKIREC) (SEQ ID NO:74). The peptide was synthesized by GenScript (Piscataway, NJ) and QC-checked by the company using MS and HPLC to have purity of at least 85%. It was used to immunize three C57BL/6 mice by MonoBoost immunization strategy by the GenScript company. Successful immunization was evaluated by indirect ELISA. Next, fresh splenocytes from successfully immunized mice were subjected to cell fusion to obtain hybridoma clonal cells. The supernatants from the hybridoma clonal cells were subjected to indirect ELISA to screen for potential positive clones. 20 positive clones (1F1, 3B5, 4D9, 4E4, 4H8, 7G2, 8H9, 9H10, 13C12, 13F3, 13G10, 14G4, 16B6, 16F12, 17F1, 18B8, 18H10, 19G4, 20A8, 20F10) were identified (FIG. 1A). All the clone supernatants can detect denatured and non-denatured GST-tagged human LANCL1 1-42aa peptide overexpressed in E. coli B21 lysate by Western blots (FIGS. 1B and 1C) as summarized in FIG. 1D.

To further screen for target monoclonal antibodies, they were tested on non-denatured human full-length His-tagged LANCL1 protein overexpressed in PLC/PRE/5 lysate by Western blots (FIG. 1E). It was found that both 4D9 and 19G4 were able to detect non-denatured full-length human LANCL1 protein in Western blots, with the latter showing stronger intensity of the detected bands (FIG. 1E). It was also found that 4D9 was only able to weakly detect non-denatured human LANCL1 protein but unable to detect denatured human LANCL1 protein; while 19G4 could strongly detect both non-denatured and denatured human LANCL1 protein (FIG. 1F). Based on this, the 4D9 and 19G4 hybridoma cells were subjected to further subcloning and expansion to purify the corresponding 4D9 and 19G4 monoclonal antibodies by the GenScript company. To this end, 4D9 and 19G4 were identified as target monoclonal antibodies for further functional validation experiments.

### Example 2. Functional Validation of LANCL1 Monoclonal Antibodies 4D9 and 1964

LANCL1 monoclonal antibodies 4D9 and 19G4 were tested on sphere formation assays, which are gold standard in vitro tests for the functionality of liver tumor-initiating cells (LTICs). Both 4D9 and 1964 significantly suppressed the sphere forming ability of PLC/PRF/5 cells at 3200 ng/100 μl dosage in 96-well seeded with 700 PLC/PRF/5 cells (FIG. 2). PLC/PRF/5 is an Alexander cell line that was isolated from the liver of a donor with hepatitis B virus-positive hepatoma. Higher dosage of 6400 ng and 12800 ng/100 μl resulted in more severe suppression on sphere formation as compared to the corresponding IgG controls.

The suppressive effects of 4D9 and 19G4 on tumor growth in vivo was tested. PLC/PRF/5 HCC cells in 0.5× growth factor-reduced Matrigel were subcutaneously injected at 5e5 cells per site on a single flank of each NOD-SCID mouse. When palpable tumors were observed on day 9 after the PLC/PRF/5 cell injection, the first doses of the monoclonal antibodies and the corresponding IgG control (ctrl) were injected intraperitoneally at 12 mg/kg to respective groups of mice. Two doses were administered per week for two weeks. The mouse weights were not affected by the administration of the 4D9 and 19G4 monoclonal antibodies as compared to the IgG control (ctrl) (FIG. 3A). 19G4 significantly suppressed the tumor size as compared to the IgG ctrl as observed on Day 19 after the PLC/PRF/5 cell injection, while 4D9 also showed suppressive trend but without sufficient statistical significance (FIG. 3B). Tumors collected at endpoint (Day 21 after PLC/PRF/5 cell injection) showed reduced tumor sizes for 4D9 and 19G4 monoclonal antibody treatment as compared to the IgG control (ctrl) (FIG. 3C). Overall, 4D9 and 19G4 monoclonal antibodies suppressed pro-tumorigenic features of HCC cells with the latter 19G4 showing more pronounced effects.

### Example 3. Hybridoma Sequencing to Obtain Amino Acid Sequences for LANCL1 Monoclonal Antibodies 4D9 and 19G4

To obtain the DNA sequences encoding the corresponding amino acid sequences for the respective human LANCL monoclonal antibodies 4D9 and 19G4, the protocol by Meyer et al. (5) and the following methods as adopted by the GenScript company were used. In brief, total RNA was isolated from the hybridoma clonal cells following the technical manual of RNA Isolation Kit (Vazyme, Catalog No. RC112-01). Total RNA was then reverse-transcribed into cDNA using isotype-specific anti-sense primers or universal primers following the technical manual of SMARTScribe Reverse Transcriptase (TaKaRa, Catalog No. 639536). The antibody fragments of VH and VL were amplified according to the standard operating procedure (SOP) of rapid amplification of cDNA ends (RACE) of GenScript. Amplified antibody fragments were cloned into a standard cloning vector separately. Colony PCR was performed to screen for clones with inserts of correct sizes. No less than five colonies with inserts of correct sizes were sequenced for each fragment. The sequences of different clones were aligned and the consensus sequences of these clones are provided as SEQ ID NO:3, 4, 19, and 20.

It was found that the heavy (H) chains of 4D9 and 19G4 mainly differ in CDR2 and CDR3 regions, with some minor difference in the FR3 and FR4 regions (see SEQ ID NOs: 3 and 4). For the light (L) chains of 4D9 and 19G4, there was difference in the signal peptide, CDR2, FR3, and CDR3 regions between them; while there was minor difference in the FR1, CDR1, FR2 and FR4 regions between them (see SEQ ID NOs: 19 and 20).

### 4. LANCL1 Monoclonal Antibody 19G4 Suppressed Orthotopic HCC Growth and Lung Metastasis in Mouse HCC Model.

Further in vivo functional tests in a mouse orthotopic HCC model were conducted using the LANCL1 monoclonal antibody 19G4 and on HCC cells in vitro, using the LANCL1 monoclonal antibody 19G4.

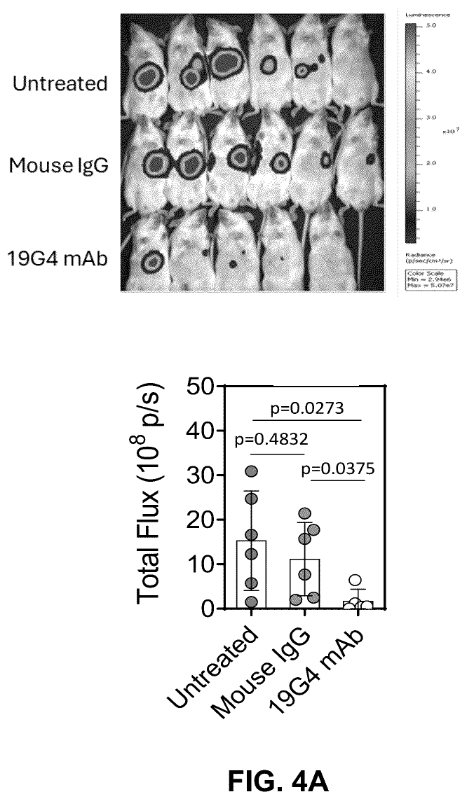

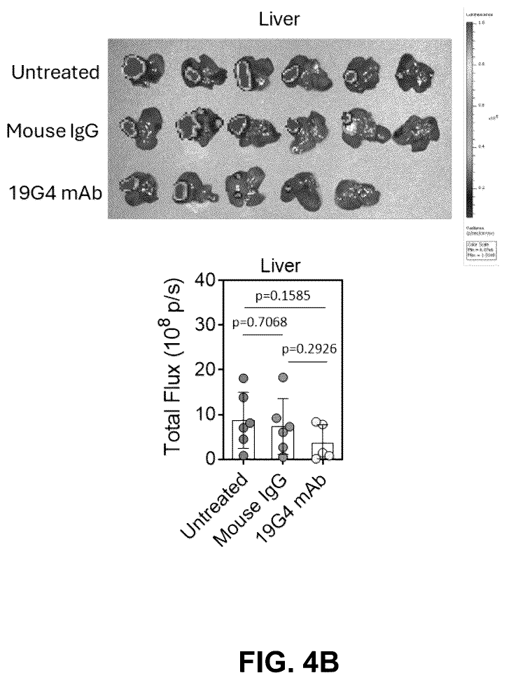

For the orthotopic experiment, 1×10⁶ luciferase-labelled PLC/PRF/5 HCC cells in 1× Matrigel were injected into the left lobe of the liver of each NOD-SCID mouse. When tumor formation was observed (by luciferase signal imaging at 2 weeks after the orthotopic injection), the mice were randomized into respective groups to receive the 19G4 monoclonal antibody, the corresponding mouse IgG control or no treatment. The antibodies were injected intraperitoneally at 60 mg/kg. Two doses were administered per week for two weeks. Liver tumor size was indicated by luciferase signal imaging of the mice and the extracted livers respectively at end point. Lung metastasis of the PLC/PRF/5 HCC cells was assessed by the luciferase signal in the extracted lung tissues. These studies showed that the 19G4 monoclonal antibody significantly suppressed liver tumor growth and lung metastasis as compared to the IgG ctrl (FIG. 4A-4C). 5. LANCL1 monoclonal antibody 19G4 promoted the cytotoxicity of HCC cells and inhibited the migration and invasion of HCC cells in vitro.

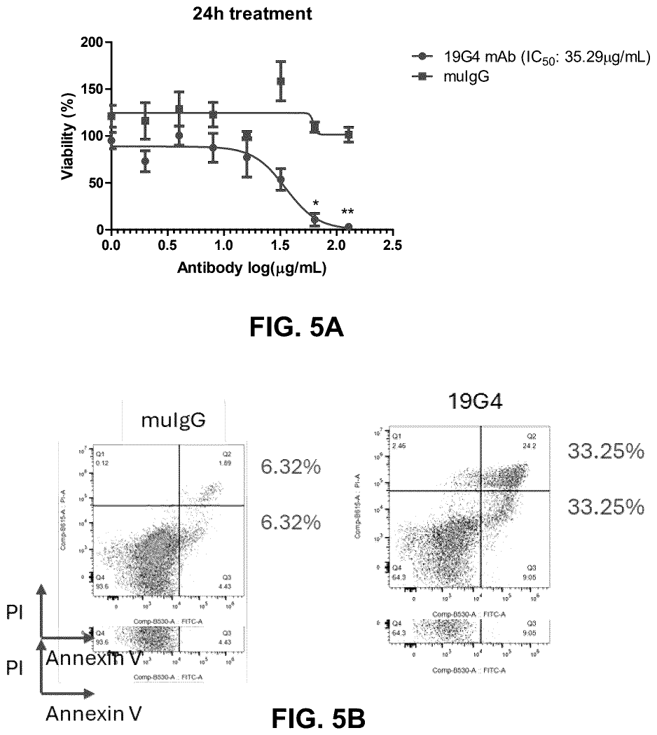

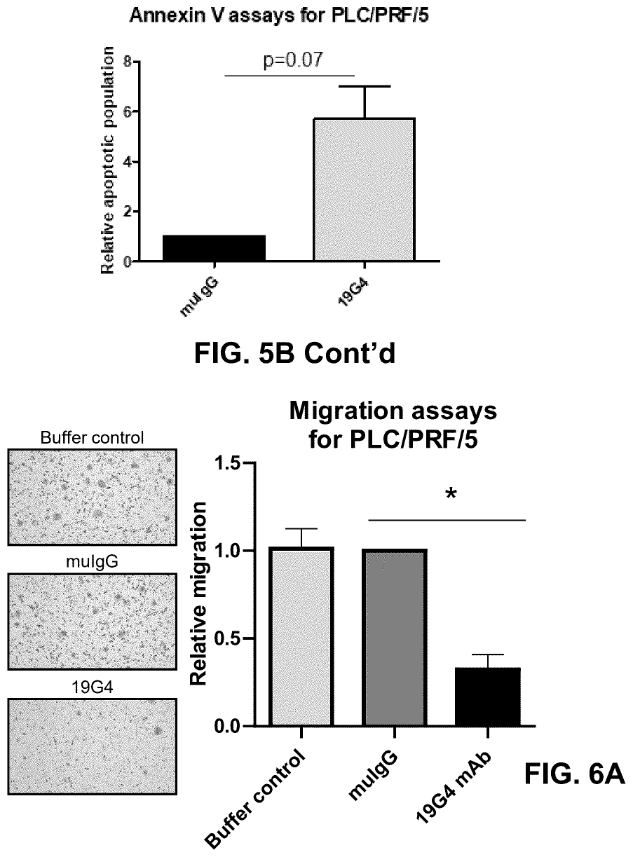

Cytotoxic activity of 19G4 monoclonal antibody on liver cancer cells was assessed using cytotoxicity assays to determine its IC50 relative to the IgG control and the Annexin V assays by flow cytometry to assess the effects of the antibody on the apoptosis of the HCC cells. For the cytotoxicity assays, 2,000 PLC/PRF/5 cells were seeded per well on a 96-well plate and the different wells of PLC/PRF/5 cells were treated with eight different indicated concentrations of the 19G4 monoclonal antibody or the respective IgG control. After 24 hours, cell viability was assessed using DAPI staining and subsequent cell counting. The IC50 value of 19G4 monoclonal antibody was determined to be 35.29 μg/ml, while the IgG control failed to inhibit the growth of HCC cells (FIG. 5A). For Annexin V/PI staining assays, cells were seeded at 10,000 cells per well on a 24-well plate with four wells for each condition, and immediately after cell seeding, the wells of cells were treated with 128 μg/ml of 19G4 monoclonal antibody or the IgG control respectively. A total of 1×10⁵ PLC/PRF/5 cells were harvested for each condition for flow cytometry, with 10,000 events analyzed per sample. A 48-hour treatment of the 19G4 monoclonal antibody resulted increased apoptosis which was not statistically significant, when compared to the IgG control (FIG. 5B).

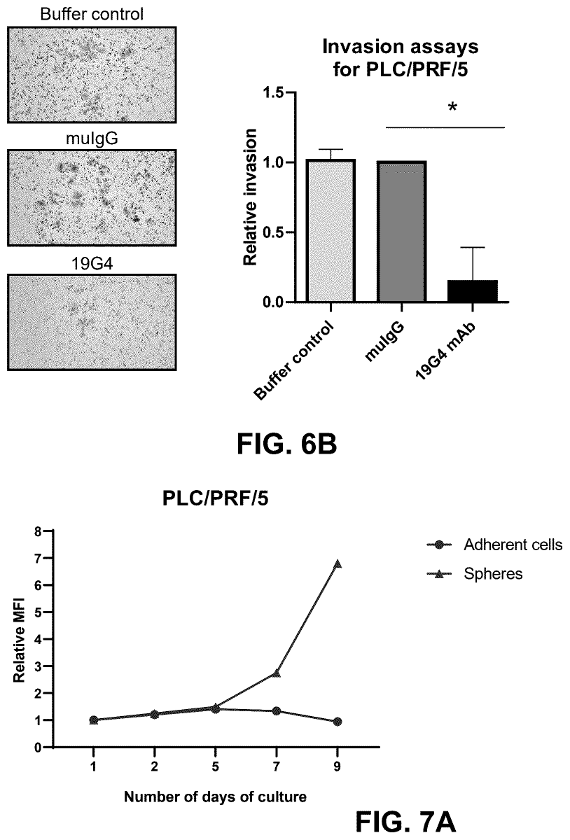

Besides cytotoxicity, the effects of the LANCL1 19G4 antibody on the migratory and invasive ability of the HCC cells were also investigated, by using transwell migration and invasion assays respectively. In the transwell migration assays, 1×10⁵ PLC/PRF/5 cells were seeded in the upper chamber, with 128 μg/mL of the 19G4 monoclonal antibody applied to both the upper and lower chambers. The lower chamber contained 10% FBS as a chemoattractant whereas the upper chamber had serum-free medium. After 6 hours of incubation at 37° C., the number of cells that had migrated across the membrane to the bottom surface of the membrane in the transwell were stained and counted in random views. 19G4 antibody significantly reduced HCC cell migration as compared to the IgG control (FIG. 6A). In the transwell invasion assays, the upper chamber was pre-coated with 3 mg/ml Matrigel before seeding of 5×10⁴ PLC/PRF/5 cells. All other conditions were the same as the migration assays, except for an extended incubation period of 72 hours. The 19G4 antibody significantly decreased HCC cell invasion as compared to the IgG control (FIG. 6B). Three independent trials were performed for all the assays.

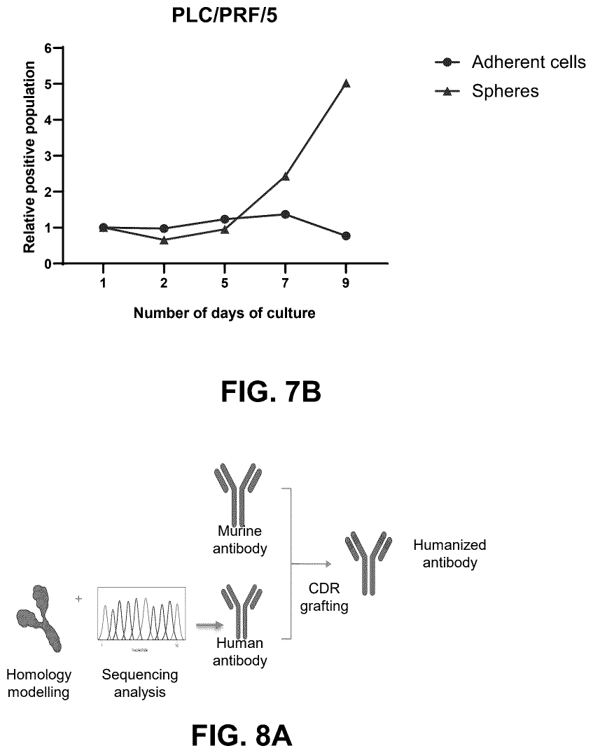

By adopting a revised staining protocol in which the cells were incubated with the LANCL1 19G4 antibody at 4° C. for 30 min before fixation by 4% paraformaldehyde for 10 min and subsequent incubation with the secondary antibody at 4° C. for 30 min, we were able to use the LANCL1 19G4 antibody for flow cytometry analysis for the cell surface expression of LANC1. Interestingly, we found that there was increase in the mean fluorescence intensity (MFI) of the total cell population (FIG. 7A) and the percentages of the cells positive for the expression of LANCL1 (FIG. 7B) for the spheroid culture but not the 2-dimensional adherent culture of PLC/PRF/5 cells over a period of 9 days. These may implicate an enrichment of LANCL1-positive cells in spheroid culture.

With the above solid functional data for the suppressive effects of the LANCL1 19G4 monoclonal antibody on HCC, subsequent studies proceeded to the humanization process of this antibody.

### 6. Humanization of the LANCL1 Monoclonal Antibody 19G4.

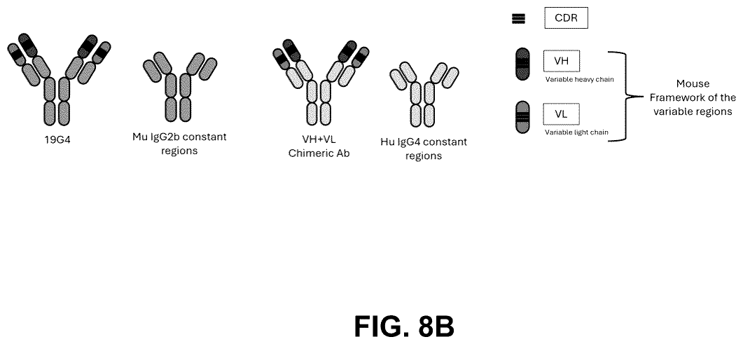

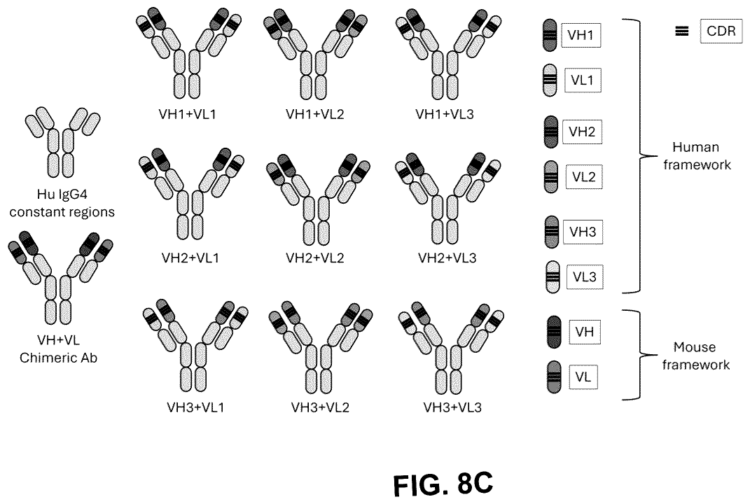

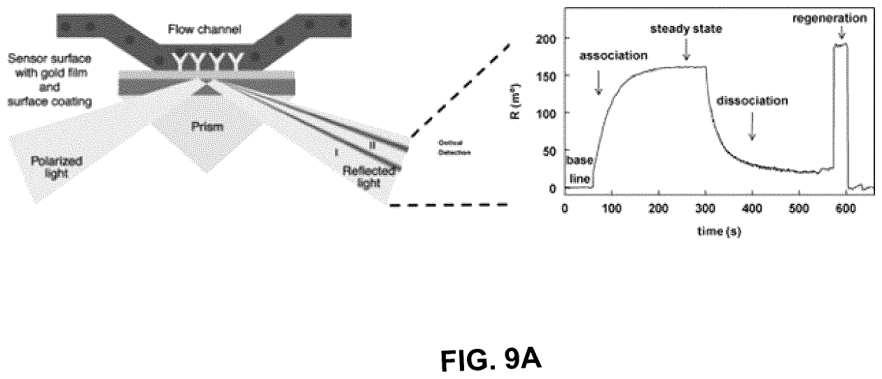

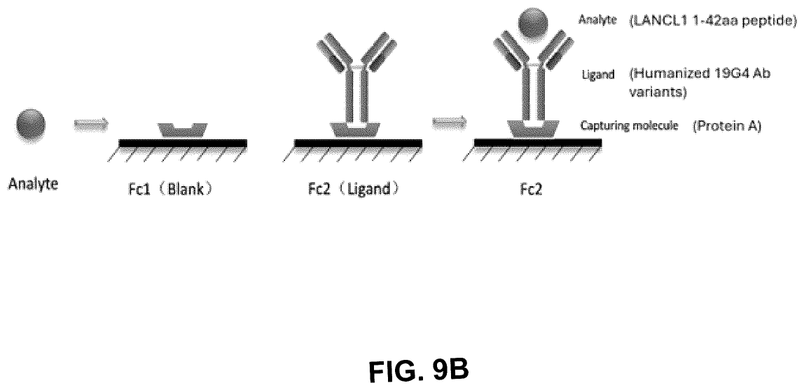

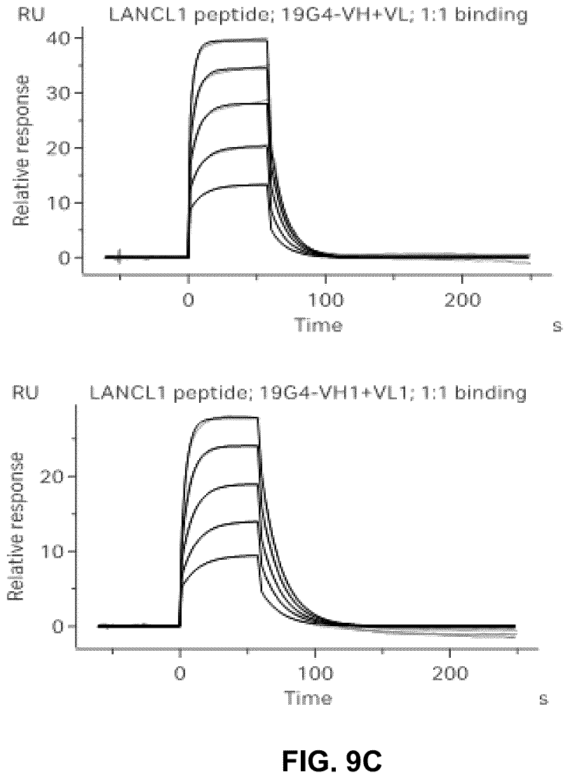

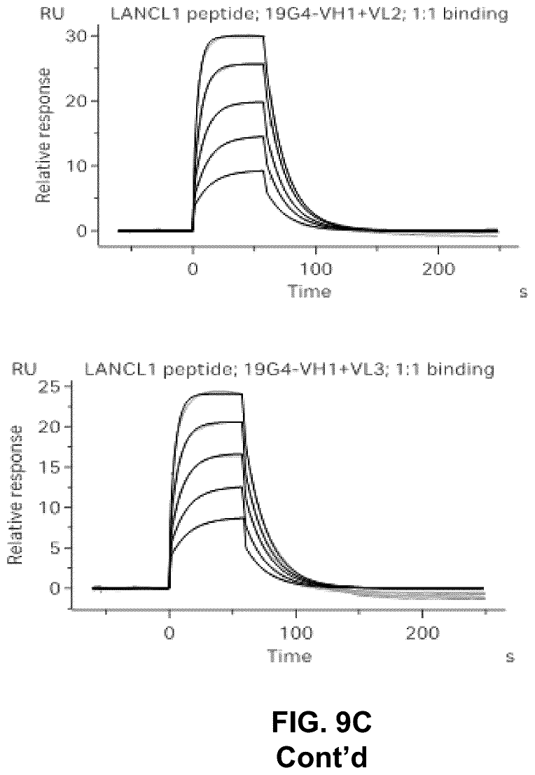

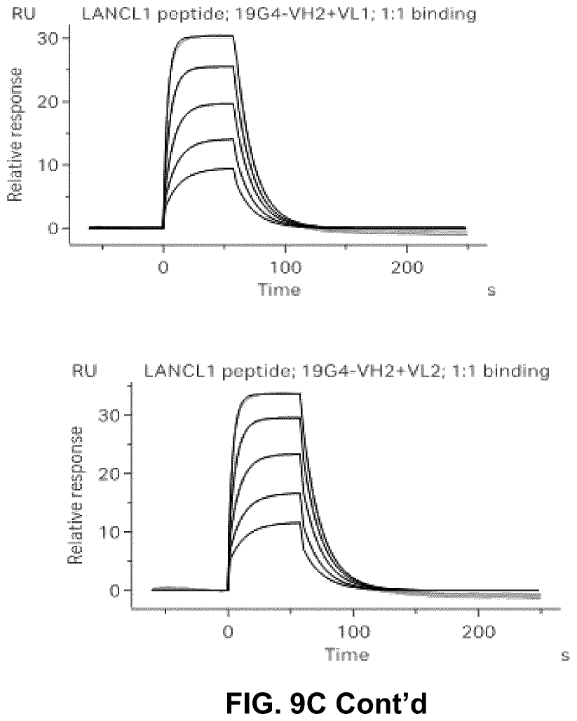

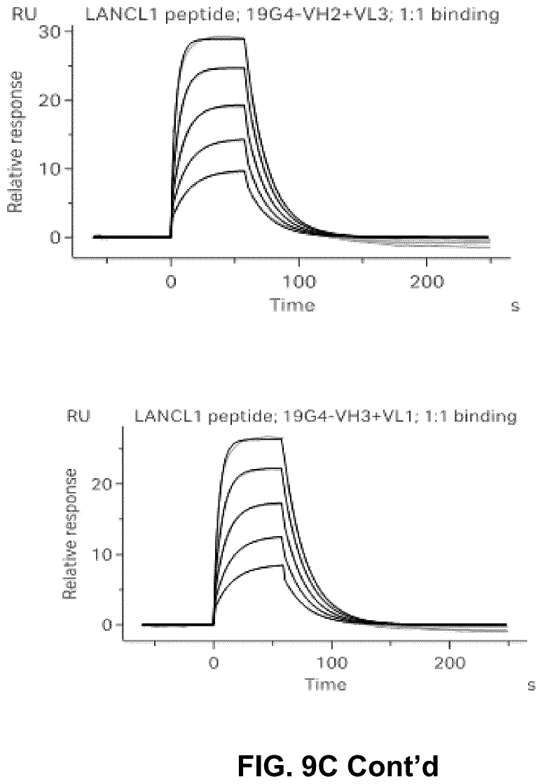

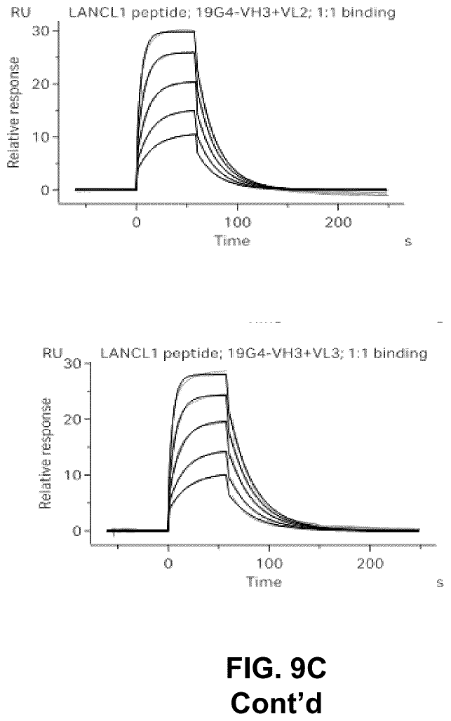

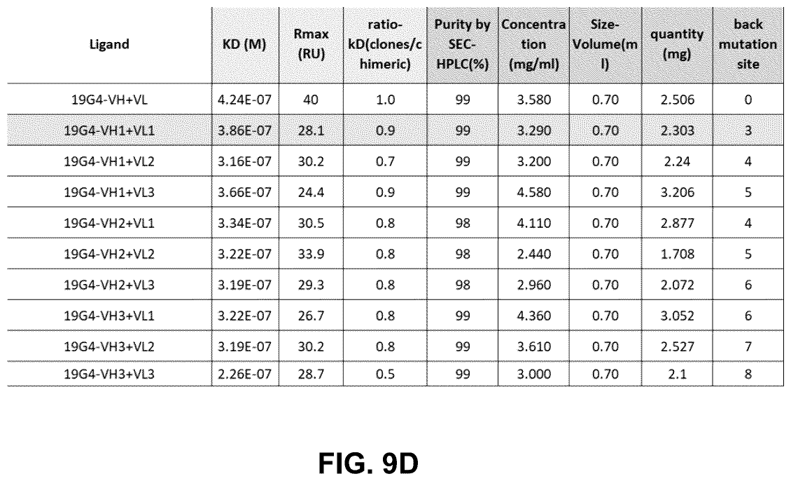

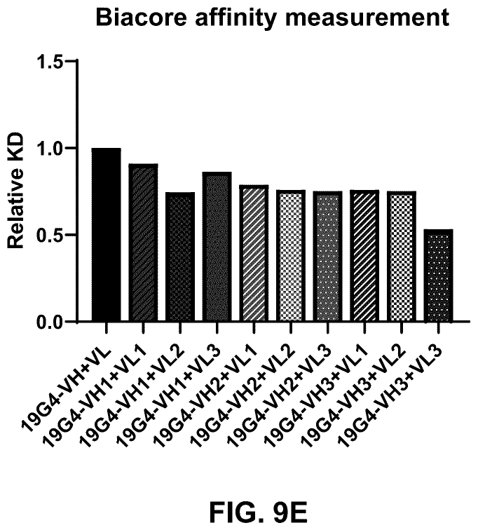

Humanized antibodies were designed using CDR grafting to human IgG4 (FIG. 8A). In brief, homology modeling of parental 19G4 antibody Fv fragments was carried out by searching the sequence against IgBLAST database to identify the best templates that share the highest sequence identities to the parental 19G4 antibody for Fv fragments for building the domain interface. The identified homology antibody sequence was selected as the human acceptor for grafting the Fv fragment of the parental 19G4 antibody (FIG. 8B). Next, by comparing the sequences of the grafted antibody and the parental antibody in the proximity of CDRs, canonical residues, loop interaction, and foundation core, key residues that are different in grafted and parental antibody sequences were identified as the putative back mutation sites so that through stepwise incorporation of one or more back mutations in the grafted antibody sequence, variant of the humanized antibody can be designed. In this way, three different variants for each of the heavy chain and light chain were obtained. These heavy and light chain variants when combined lead to 9 different combinations (i.e. variants) of the humanized Ab (FIG. 8C). These humanized antibody variants were synthesized and purified and subjected to affinity measurement by Biacore (FIG. 9A), in which the analyte, ligand and the capturing molecule used in the Biacore measurement were the LANCL1 1-42aa peptide, humanized 19G4 antibody variants and the protein A respectively (FIG. 9B). All the data were processed using the Biacore 8K Evaluation Software version 4.0. The binding sensor-grams were shown in FIG. 9C and the binding measurement data was shown in FIG. 9D. All of the antibody variants have affinity within the range of 0.5 to 1.0 relative to that of the chimeric humanized 19G4 antibody VH+VL, with the lowest being VH3+VL3 (FIG. 9E). The amino acid sequences of the variable regions of both the heavy and light chains for the chimeric humanized 19G4 antibody 9 and the various humanized antibody variants 9 are provided here (below).

H chain variable region of the chimeric humanized antibody:

(SEQ ID NO: 75)
EVQLQQSGPVLVKPGASVKMSCKASGYTFTDYYMNWVKQSHGKSLEWI
GVINPYNGHTNYNQKFKGKATLTVDKSSNTAYMELNSLTSEDSAVYYC
ARFPYYGSSYRVDYWGQGTTLTVSS;

L chain variable region of the chimeric humanized antibody:

(SEQ ID NO: 76)
DIVLTQSPATLSVTPGDSVSLSCRASQSISNNLHWYQQKSHESPRLLI
KYASQSISGIPSRFSGSGSGTDFTLSINSVETEDFGMYFCQQINSWPL
TFGAGTKLELK;

H chain variable regions of the humanized antibody variants:

VH1:
(SEQ ID NO: 77)
QVQLVQSGAEVKKPGASVKVSCKASGYTFTDYYMNWVRQAPGQGLEWI
GVINPYNGHTNYNQKFKGRVTMTVDTSTSTVYMELSSLRSEDTAVYYC
ARFPYYGSSYRVDYWGQGTTVTVSS;
VH2:
(SEQ ID NO: 78)
QVQLVQSGAEVKKPGASVKVSCKASGYTFTDYYMNWVRQAPGQGLEWI
GVINPYNGHTNYNQKFKGRATMTVDTSTSTVYMELSSLRSEDTAVYYC
ARFPYYGSSYRVDYWGQGTTVTVSS;
VH3:
(SEQ ID NO: 79)
QVQLVQSGAEVKKPGASVKVSCKASGYTFTDYYMNWVKQAPGQGLEWI
GVINPYNGHTNYNQKFKGRATMTVDKSTSTVYMELSSLRSEDTAVYYC
ARFPYYGSSYRVDYWGQGTTVTVSS.

L chain variable regions of the humanized antibody variants:

VL1:
(SEQ ID NO: 80)
EIVMTQSPATLSVSPGERATLSCRASQSISNNLHWYQQKPGQAPRLLI
KYASQSISGIPARFSGSGSGTEFTLTISSLQSEDFAVYYCQQINSWPL
TFGGGTKLEIK;
VL2:
(SEQ ID NO: 81)
EIVMTQSPATLSVSPGERATLSCRASQSISNNLHWYQQKPGQAPRLLI
KYASQSISGIPSRFSGSGSGTEFTLTISSLQSEDFAVYYCQQINSWPL
TFGGGTKLEIK;
VL3:
(SEQ ID NO: 82)
EIVMTQSPATLSVSPGERATLSCRASQSISNNLHWYQQKPGQAPRLLI
KYASQSISGIPSRFSGSGSGTEFTLTISSLQSEDFAVYFCQQINSWPL
TFGGGTKLEIK.

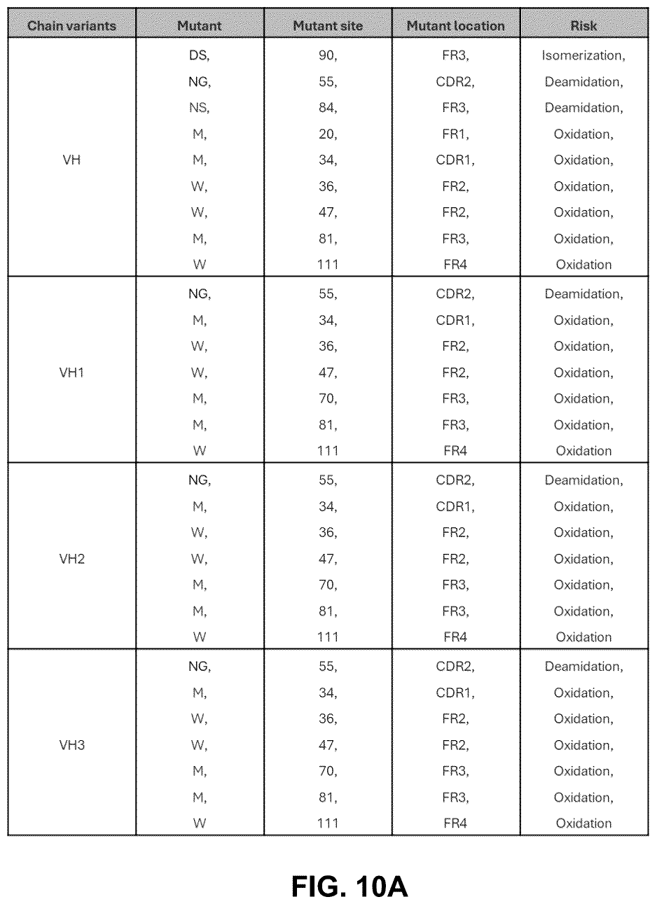

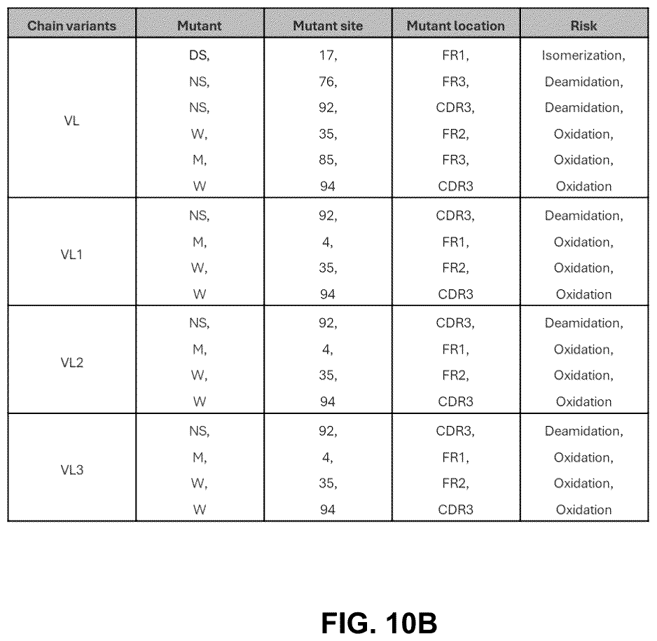

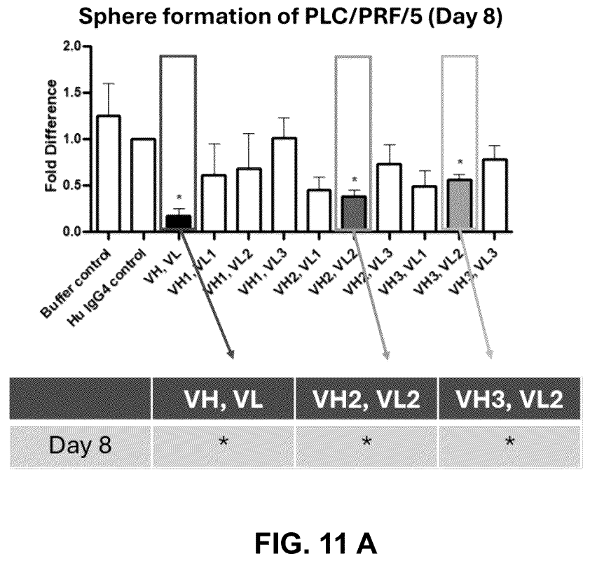

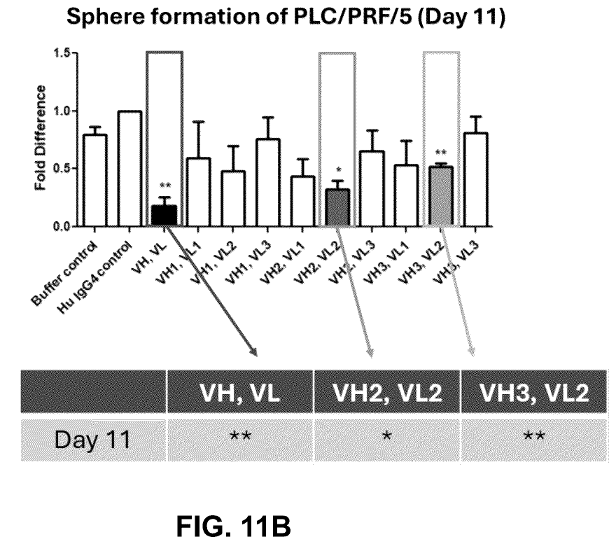

The risk of post-translational modifications (PTM) was determined for the variable regions of both the heavy (FIG. 10A) and light (FIG. 10B) chains for the chimeric humanized 19G4 antibody and the various humanized antibody variants. Most of the sequences have low PTM risk except a moderate risk, which is still acceptable, for the “NG” residues on the heavy chains of the humanized antibody variants. With the above in silico analysis data, studies further tested the functional effects of the monoclonal antibody variants on HCC cells. The data showed that the humanized antibody variants VH2+VL2 and VH3+VL2 significantly suppressed sphere formation, similar to the chimeric humanized antibody VH+VL, as indicated by the reduced number of spheres formed on Day 8 (FIG. 11A) and Day 11 (FIG. 11B) as compared to the IgG control.

### SUMMARY/CONCLUSIONS

LANCL1 monoclonal antibodies 4D9 and 19G4 were identified as having suppressive effects on HCC cells and the DNA sequences encoding the amino acid sequences of the respective monoclonal antibodies from the corresponding hybridoma clones were identified. The DNA and amino acid sequences for the monoclonal antibodies forms the basis for further engineering and improvement of the antibodies for translational applications to target LANCL1 in liver tumor-initiating cells as a means of treating hepatocellular carcinoma (HCC).

Studies herein further functionally validated the 19G4 monoclonal antibody significantly suppressed orthotopic HCC growth and lung metastasis, promoted the cytotoxicity and apoptosis of HCC cells and inhibited the migration and invasion of HCC cells. By using the antibody, enrichment of LANCL1-positive cells in HCC spheroid culture was observed as compared to 2-dimensional adherent culture. The 19G4 antibody was then subjected to humanization process and 9 variants of humanized antibodies were designed, among which, the VH2+VL2 and VH3+VL2 show significant suppressive effects on HCC cell sphere formation.

### REFERENCES

1. Yi S Y, Hao Y B, Nan K J, and Fan T L. Cancer stem cells niche: a target for novel cancer therapeutics. Cancer Treat Rev. 2013; 39:290-296.
2. Visvader J E, and Lindeman G J. Cancer stem cells in solid tumours: accumulating evidence and unresolved questions. Nat Rev Cancer. 2008; 8:755-768.
3. Tirino V, Desiderio V, Paino F, De Rosa A, Papaccio F, La Noce M, Laino L, et al. Cancer stem cells in solid tumors: an overview and new approaches for their isolation and characterization. FASEB J. 2013; 27:13-24.
4. Huang H Y*, Tsui Y M*, Ho D W, Chung C Y, Sze K M, Lee E, Cheung G C, Zhang V X, Wang X, Lyu X Y, Ng I O. LANCL1, a cell-surface protein, promotes liver tumor initiation via FAM49B-Rac1 axis to suppress oxidative stress. Hepatol. 2023; 79(2):323-340.
5. Meyer L, López T, Espinosa R, Arias C F, Vollmers C, DuBois R M. A simplified workflow for monoclonal antibody sequencing. PLOS One. 2019; 14 (6): e0218717.

It is understood that the disclosed method and compositions are not limited to the particular methodology, protocols, and reagents described as these can vary. It is also to be understood that the terminology used herein is for the purpose of describing particular embodiments only and is not intended to limit the scope of the present invention which will be limited only by the appended claims.

Those skilled in the art will recognize, or be able to ascertain, using no more than routine experimentation, many equivalents to the specific embodiments of the method and compositions described herein. Such equivalents are intended to be encompassed by the following claims.

We claim:

1. An antibody or antigen-binding fragment thereof, comprising a heavy chain variable region comprising three complementarity determining regions (CDRs) and a light chain variable region comprising three CDRs, wherein:
(i) the three heavy chain variable region CDRs comprise amino acid sequences selected from the group consisting of DYYMN (SEQ ID NO:5), DINPNNGGASYNQKFKG (SEQ ID NO:6), VINPYNGHTNYNQKFKG (SEQ ID NO:7), SGDGYYFAS (SEQ ID NO:8), FPYYGSSYRVDY (SEQ ID NO:9) respectively, or a functional variant thereof;
(ii) the three light chain variable region CDRs comprise amino acids sequences selected from the group consisting of RASKSVSTSGYSYMH (SEQ ID NO:21), RASQSISNNLH (SEQ ID NO:22), LVSNLES (SEQ ID NO:23), QHIRELT (SEQ ID NO:24), QQINSWPLT (SEQ ID NO:25), YASQSIS (SEQ ID NO:73), respectively, or a functional variant thereof; and
wherein the antibody or antigen binding fragment thereof binds to LanC Like Glutathione S-Transferase 1 (LANCL1) protein.

2. The antibody or antigen-binding fragment thereof of claim 1, wherein:
(i) the three heavy chain variable region CDRs comprise the amino acid sequences: DYYMN (SEQ ID NO:5), DINPNNGGASYNQKFKG (SEQ ID NO:6), SGDGYYFAS (SEQ ID NO:8), respectively, or a functional variant thereof, and the three light chain variable region CDRs comprise the amino acid sequences: RASKSVSTSGYSYMH (SEQ ID NO:21), LVSNLES (SEQ ID NO:23), QHIRELT (SEQ ID NO:24) respectively, or a functional variant thereof; or
(ii) the three heavy chain variable region CDRs comprise the amino acid sequences: DYYMN (SEQ ID NO:5), VINPYNGHTNYNQKFKG (SEQ ID NO:7), FPYYGSSYRVDY (SEQ ID NO:9) respectively, or a functional variant thereof, and the three light chain variable region CDRs comprise amino acid sequences: RASQSISNNLH (SEQ ID NO:22), YASQSIS (SEQ ID NO:73), QQINSWPLT (SEQ ID NO:25), respectively, or a functional variant thereof.

3. The antibody or antigen-binding fragment of claim 1, wherein:
(a) the three heavy chain variable region CDRs comprise amino acids SEQ ID NO:5, SEQ ID NO:6, SEQ ID NO:8, respectively or a functional variant thereof, and the three light chain variable region CDRs comprise amino acids SEQ ID NO:21, SEQ ID NO:23, SEQ ID NO:24 respectively, or a functional variant thereof;
(b) the three heavy chain variable region CDRs comprise amino acids SEQ ID NO:5, SEQ ID NO:7, SEQ ID NO:9 respectively, or a functional variant thereof, and the three light chain variable region CDRs comprise amino acids SEQ ID NO:22, SEQ ID NO:73, SEQ ID NO:25, respectively, or a functional variant thereof;
(c) the three heavy chain variable region CDRs comprise amino acids SEQ ID NO:5, SEQ ID NO:7, SEQ ID NO:9 respectively, or a functional variant thereof, and the three light chain variable region CDRs comprise amino acids SEQ ID NO:21, SEQ ID NO:23, SEQ ID NO:24 respectively, or a functional variant thereof; or
(d) the three heavy chain variable region CDRs comprise amino acids SEQ ID NO:5, SEQ ID NO:6, SEQ ID NO:8 respectively, or a functional variant thereof, and the three light chain variable region CDRs comprise amino acids SEQ ID NO:22, SEQ ID NO:73, SEQ ID NO:25, respectively, or a functional variant thereof.

4. The antibody or antigen-binding fragment of claim 1, wherein the functional variant has about 70%, about 75%, about 80%, about 85%, about 90%, about 95% or about 99% sequence identity to SEQ ID NO:5, SEQ ID NO:6, SEQ ID NO:8, SEQ ID NO:7, SEQ ID NO:9, SEQ ID NO:21, SEQ ID NO:23, SEQ ID NO:24, SEQ ID NO:22, SEQ ID NO:73 or SEQ ID NO:25.

5. The antibody or antigen-binding fragment of claim 1, comprising a light chain variable region comprising the amino acid sequence of SEQ ID NO:19 and a heavy chain variable region comprising the amino acid sequence of SEQ ID NO:3.

6. The antibody or antigen-binding fragment of claim 1, comprising (a) a light chain variable region comprising the amino acid sequence of SEQ ID NO:20 and a heavy chain variable region comprising the amino acid sequence of SEQ ID NO:4; b) a light chain variable region comprising the amino acid sequence of SEQ ID NO:19 and a heavy chain variable region comprising the amino acid sequence of SEQ ID NO:4 or (c) a light chain variable region comprising the amino acid sequence of SEQ ID NO:20 and a heavy chain variable region comprising the amino acid sequence of SEQ ID NO:3.

7. The antibody or antigen-binding fragment of claim 1, comprising a humanized light chain variable region comprising the amino acid sequence of SEQ ID NO:76 or a functional variant thereof and a humanized heavy chain variable region comprising the amino acid sequence of SEQ ID NO:75 or a functional variant thereof.

8. The antibody or antigen-binding fragment of claim 11, wherein the chain variable region variant comprises: (a) the amino acid sequence of SEQ ID NO:80, 81 or 82 and/or (b) the heavy chain variable region variant comprises the amino acid sequence of SEQ ID NO:77, 78 or 79.

9. The antibody or antigen binding fragment thereof of claim 1, comprising one or more constant domains from an immunoglobulin constant region (Fc), wherein the constant domains are human constant domains selected from the group consisting of IgA, IgD, IgE, IgG, or IgM, optionally, wherein human IgG constant domain is selected from the group consisting of IgG1, IgG2, IgG3, or IgG4.

10. The antibody or antigen-binding fragment of claim 1, wherein the antibody is a monoclonal antibody, a human antibody, a chimeric antibody, or a humanized antibody.

11. A humanized antibody comprising one or more human IgG constant domains, a heavy chain variable region comprising three complementarity determining regions (CDRs) and a light chain variable region comprising three CDRs,
comprising (a) a humanized light chain variable region comprising the an amino acid sequence selected from the group consisting of SEQ ID NO:76, 80, 81 or 82 and a humanized heavy chain variable region comprising the amino acid sequence of SEQ ID NO:75, 77, 78 or 79,
wherein:
(i) each heavy chain variable region comprises the three heavy chain variable region CDRs comprising amino acid sequences selected from the group consisting of: DYYMN (SEQ ID NO:5), DINPNNGGASYNQKFKG (SEQ ID NO:6), VINPYNGHTNYNQKFKG (SEQ ID NO:7), SGDGYYFAS (SEQ ID NO:8), FPYYGSSYRVDY (SEQ ID NO:9) or a functional variant thereof;
(ii) each light chain variable region comprises the three light chain variable region CDRs comprise amino acid sequences selected from the group consisting of: RASKSVSTSGYSYMH (SEQ ID NO:21), RASQSISNNLH (SEQ ID NO:22), LVSNLES (SEQ ID NO:23), QHIRELT (SEQ ID NO:24), YASQSIS (SEQ ID NO:73), QQINSWPLT (SEQ ID NO:25), or a functional variant thereof; and
wherein the humanized antibody or antigen binding fragment thereof binds to human LanC Like Glutathione S-Transferase 1 (LANCL1) protein.

12. The humanized antibody of claim 11, wherein:
(i) the three heavy chain variable region CDRs comprise amino acids DYYMN (SEQ ID NO:5), DINPNNGGASYNQKFKG (SEQ ID NO:6), SGDGYYFAS (SEQ ID NO:8), respectively, or DYYMN (SEQ ID NO:5), VINPYNGHTNYNQKFKG (SEQ ID NO:7), FPYYGSSYRVDY (SEQ ID NO:9) respectively, or a functional variant thereof; and
(ii) the three light chain variable region CDRs comprise amino acid sequences: RASKSVSTSGYSYMH (SEQ ID NO:21), LVSNLES (SEQ ID NO:23), QHIRELT (SEQ ID NO:24) respectively, or RASQSISNNLH (SEQ ID NO:22), YASQSIS (SEQ ID NO:73), QQINSWPLT (SEQ ID NO:25), respectively, or a functional variant thereof.

13. The humanized antibody of claim 12, wherein:
(i) the three heavy chain variable region CDRs comprise the amino acid sequences: DYYMN (SEQ ID NO:5), DINPNNGGASYNQKFKG (SEQ ID NO:6), SGDGYYFAS (SEQ ID NO:8), respectively, or a functional variant thereof, and the three light chain variable region CDRs comprise the amino acid sequences: RASKSVSTSGYSYMH (SEQ ID NO:21), LVSNLES (SEQ ID NO:23), QHIRELT (SEQ ID NO:24) respectively, or a functional variant thereof; or
(ii) the three heavy chain variable region CDRs comprise the amino acid sequences: DYYMN (SEQ ID NO:5), VINPYNGHTNYNQKFKG (SEQ ID NO:7), FPYYGSSYRVDY (SEQ ID NO:9) respectively, or a functional variant thereof, and the three light chain variable region CDRs comprise amino acid sequences: RASQSISNNLH (SEQ ID NO:22), YASQSIS (SEQ ID NO:73), QQINSWPLT (SEQ ID NO:25), respectively, or a functional variant thereof.

14. The humanized antibody of 13, wherein:
(a) the three heavy chain variable region CDRs comprise SEQ ID NO:5, SEQ ID NO:6, SEQ ID NO:8, respectively or a functional fragment thereof, and the three light chain variable region CDRs comprise SEQ ID NO:21, SEQ ID NO:23, SEQ ID NO:24 respectively, or a functional fragment thereof;
(b) the three heavy chain variable region CDRs comprise SEQ ID NO:5, SEQ ID NO:7, SEQ ID NO:9 respectively, or a functional fragment thereof, and the three light chain variable region CDRs comprise SEQ ID NO:22, SEQ ID NO:73, SEQ ID NO:25, respectively, or a functional fragment thereof; or
(c) the three heavy chain variable region CDRs comprise SEQ ID NO:5, SEQ ID NO:6, SEQ ID NO:8 respectively, or a functional fragment thereof, and the three light chain variable region CDRs comprise SEQ ID NO:22, SEQ ID NO:73, SEQ ID NO:25, respectively, or a functional fragment thereof.

15. A nucleic acid encoding the antibody or antigen-binding fragment of claim 14.

16. A pharmaceutical composition comprising the antibody or antigen-binding fragment of claim 1, optionally wherein the antibody or fragment thereof is a humanized antibody, and one or more pharmaceutically acceptable carriers and/or excipients.

17. A method of treating a subject in need thereof, comprising administering to the subject, an effective amount of the pharmaceutical composition of claim 25, optionally, the subject has a disease or disorder caused by or characterized by increased presence of LanC Like Glutathione S-Transferase 1 (LANCL1) protein or a fragment thereof, wherein the pharmaceutical composition is effective to reduce the proliferation of tumor-initiating cells, sphere formation of hepatocellular carcinoma cells, and/or blocking or reducing the activity of LANCL1.

18. The method of claim 16, wherein the subject has liver cancer or is at risk of developing liver cancer.

19. The method of claim 16, wherein the liver cancer is hepatocellular carcinoma.

20. The method of claim 16, wherein the pharmaceutical composition is delivered via injection or infusion.


SEQUENCE LISTING (82 sequences)

SEQ ID NO: 1 - protein, 399 aa, Homo sapiens
MAQRAFPNPYADYNKSLAEGYFDAAGRLTPEFSQRLTNKIRELLQQMERGLKSADPRDGTGYTGWAGIAVLYLHLYDVFGDPAYLQLAHGYVKQSLNCLTKRSITFLCGDAGPLAVAAVLYHKMNNEKQAEDCITRLIHLNKIDPHAPNEMLYGRIGYIYALLFVNKNFGVEKIPQSHIQQICETILTSGENLARKRNFTAKSPLMYEWYQEYYVGAAHGLAGIYYYLMQPSLQVSQGKLHSLVKPSVDYVCQLKFPSGNYPPCIGDNRDLLVHWCHGAPGVIYMLIQAYKVFREEKYLCDAYQCADVIWQYGLLKKGYGLCHGSAGNAYAFLTLYNLTQDMKYLYRACKFAEWCLEYGEHGCRTPDTPFSLFEGMAGTIYFLADLLVPTKARFPAFEL

SEQ ID NO: 2 - protein, 42 aa, synthetic construct
MAQRAFPNPYADYNKSLAEGYFDAAGRLTPEFSQRLTNKIRE

SEQ ID NO: 3 - protein, 473 aa, synthetic construct
MGWSWIFLFLLSGTAGVLSEVQLQQSGPELVKPGASVKISCKASGYTFTDYYMNWVKQSHGKSLEWIGDINPNNGGASYNQKFKGKATLTVDKSSSTAYMELRSLTSEDSAVYYCVRSGDGYYFASWGQGTLLTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 4 - protein, 476 aa, synthetic construct
MGWSWIFLFLLSGTAGVHSEVQLQQSGPVLVKPGASVKMSCKASGYTFTDYYMNWVKQSHGKSLEWIGVINPYNGHTNYNQKFKGKATLTVDKSSNTAYMELNSLTSEDSAVYYCARFPYYGSSYRVDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 5 - protein, 5 aa, synthetic construct
DYYMN

SEQ ID NO: 6 - protein, 17 aa, synthetic construct
DINPNNGGASYNQKFKG

SEQ ID NO: 7 - protein, 17 aa, synthetic construct
VINPYNGHTNYNQKFKG

SEQ ID NO: 8 - protein, 9 aa, synthetic construct
SGDGYYFAS

SEQ ID NO: 9 - protein, 12 aa, synthetic construct
FPYYGSSYRVDY

SEQ ID NO: 10 - protein, 30 aa, synthetic construct
EVQLQQSGPELVKPGASVKISCKASGYTFT

SEQ ID NO: 11 - protein, 30 aa, synthetic construct
EVQLQQSGPVLVKPGASVKMSCKASGYTFT

SEQ ID NO: 12 - protein, 14 aa, synthetic construct
WVKQSHGKSLEWIG

SEQ ID NO: 13 - protein, 32 aa, synthetic construct
KATLTVDKSSSTAYMELRSLTSEDSAVYYCVR

SEQ ID NO: 14 - protein, 32 aa, synthetic construct
KATLTVDKSSNTAYMELNSLTSEDSAVYYCAR

SEQ ID NO: 15 - protein, 11 aa, synthetic construct
WGQGTLLTVSA

SEQ ID NO: 16 - protein, 11 aa, synthetic construct
WGQGTTLTVSS

SEQ ID NO: 17 - protein, 19 aa, synthetic construct
MGWSWIFLFLLSGTAGVLS

SEQ ID NO: 18 - protein, 19 aa, synthetic construct
MGWSWIFLFLLSGTAGVHS

SEQ ID NO: 19 - protein, 228 aa, synthetic construct
MGTAALGSRFHWDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 234 aa, synthetic construct
MVFTPQILGLMLFWISASRGDIVLTQSPATLSVTPGDSVSLSCRASQSISNNLHWYQQKSHESPRLLIKYASQSISGIPSRFSGSGSGTDFTLSINSVETEDFGMYFCQQINSWPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 15 aa, synthetic construct
RASKSVSTSGYSYMH

SEQ ID NO: 22 - protein, 11 aa, synthetic construct
RASQSISNNLH

SEQ ID NO: 23 - protein, 7 aa, synthetic construct
LVSNLES

SEQ ID NO: 24 - protein, 7 aa, synthetic construct
QHIRELT

SEQ ID NO: 25 - protein, 9 aa, synthetic construct
QQINSWPLT

SEQ ID NO: 26 - protein, 23 aa, synthetic construct
DIVLTQSPASLAVSLGQRATISY

SEQ ID NO: 27 - protein, 23 aa, synthetic construct
DIVLTQSPATLSVTPGDSVSLSC

SEQ ID NO: 28 - protein, 15 aa, synthetic construct
WNQQKPGQPPRLLIY

SEQ ID NO: 29 - protein, 15 aa, synthetic construct
WYQQKSHESPRLLIK

SEQ ID NO: 30 - protein, 32 aa, synthetic construct
GVPARFSGSGSGTDFTLNIHPVEEEDAATYYC

SEQ ID NO: 31 - protein, 32 aa, synthetic construct
GIPSRFSGSGSGTDFTLSINSVETEDFGMYFC

SEQ ID NO: 32 - protein, 10 aa, synthetic construct
FGGGTKLEIK

SEQ ID NO: 33 - protein, 10 aa, synthetic construct
FGAGTKLELK

SEQ ID NO: 34 - protein, 12 aa, synthetic construct
MGTAALGSRFHW

SEQ ID NO: 35 - protein, 20 aa, synthetic construct
MVFTPQILGLMLFWISASRG

SEQ ID NO: 36 - protein, 336 aa, synthetic construct
AKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 37 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 38 - DNA, 1422 bp, synthetic construct
  source 1..1422: mol_type = other DNA
atgggatggagctggatctttctctttctcctgtcaggaactgcaggtgtcctctctgaggtccagttgcaacaatctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtgctagctacaaccagaagttcaagggcaaggccacattgactgtagacaagtcctccagcacagcctacatggagctccgcagcctgacatctgaggactctgcagtctattactgtgtaagatcgggcgatggttactactttgcttcctggggccaagggactctgctcactgtctctgcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 39 - DNA, 1431 bp, synthetic construct
  source 1..1431: mol_type = other DNA
atgggatggagctggatctttctcttcctcctgtcaggaactgcaggtgtccactctgaggtccagctgcaacagtctggacctgtgctggtgaagcctggggcttcagtgaagatgtcctgtaaggcttctggatacacattcactgactactatatgaactgggtgaagcagagccatggaaagagccttgagtggattggagttattaatccttacaacggtcatactaactacaaccagaagttcaagggcaaggccacattgactgttgacaagtcctccaacacagcctacatggagctcaacagtctgacatctgaggactctgcagtctattactgtgcaagattcccttactacggtagtagctatagggttgactactggggccaaggcaccactctcacagtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 40 - DNA, 15 bp, synthetic construct
  source 1..15: mol_type = other DNA
gactactacatgaac

SEQ ID NO: 41 - DNA, 15 bp, synthetic construct
  source 1..15: mol_type = other DNA
gactactatatgaac

SEQ ID NO: 42 - DNA, 51 bp, synthetic construct
  source 1..51: mol_type = other DNA
gatattaatcctaacaatggtggtgctagctacaaccagaagttcaagggc

SEQ ID NO: 43 - DNA, 51 bp, synthetic construct
  source 1..51: mol_type = other DNA
gttattaatccttacaacggtcatactaactacaaccagaagttcaagggc

SEQ ID NO: 44 - DNA, 27 bp, synthetic construct
  source 1..27: mol_type = other DNA
tcgggcgatggttactactttgcttcc

SEQ ID NO: 45 - DNA, 36 bp, synthetic construct
  source 1..36: mol_type = other DNA
ttcccttactacggtagtagctatagggttgactac

SEQ ID NO: 46 - DNA, 90 bp, synthetic construct
  source 1..90: mol_type = other DNA
gaggtccagttgcaacaatctggacctgagctggtgaagcctggggcttcagtgaagatatcctgtaaggcttctggatacacgttcact

SEQ ID NO: 47 - DNA, 90 bp, synthetic construct
  source 1..90: mol_type = other DNA
gaggtccagctgcaacagtctggacctgtgctggtgaagcctggggcttcagtgaagatgtcctgtaaggcttctggatacacattcact

SEQ ID NO: 48 - DNA, 42 bp, synthetic construct
  source 1..42: mol_type = other DNA
tgggtgaagcagagccatggaaagagccttgagtggattgga

SEQ ID NO: 49 - DNA, 96 bp, synthetic construct
  source 1..96: mol_type = other DNA
aaggccacattgactgtagacaagtcctccagcacagcctacatggagctccgcagcctgacatctgaggactctgcagtctattactgtgtaaga

SEQ ID NO: 50 - DNA, 96 bp, synthetic construct
  source 1..96: mol_type = other DNA
aaggccacattgactgttgacaagtcctccaacacagcctacatggagctcaacagtctgacatctgaggactctgcagtctattactgtgcaaga

SEQ ID NO: 51 - DNA, 33 bp, synthetic construct
  source 1..33: mol_type = other DNA
tggggccaagggactctgctcactgtctctgca

SEQ ID NO: 52 - DNA, 33 bp, synthetic construct
  source 1..33: mol_type = other DNA
tggggccaaggcaccactctcacagtctcctca

SEQ ID NO: 53 - DNA, 57 bp, synthetic construct
  source 1..57: mol_type = other DNA
atgggatggagctggatctttctctttctcctgtcaggaactgcaggtgtcctctct

SEQ ID NO: 54 - DNA, 57 bp, synthetic construct
  source 1..57: mol_type = other DNA
atgggatggagctggatctttctcttcctcctgtcaggaactgcaggtgtccactct

SEQ ID NO: 55 - DNA, 690 bp, synthetic construct
  source 1..690: mol_type = other DNA
atgggtactgctgctctgggttccaggttccactggtgacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 56 - DNA, 705 bp, synthetic construct
  source 1..705: mol_type = other DNA
atggttttcacacctcagatacttggacttatgcttttttggatttcagcctccagaggtgatattgtgctaactcagtctccagccaccctgtctgtgactccaggagatagcgtcagtctttcctgcagggccagccaaagtattagcaacaacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggaatgtatttctgtcaacaaattaacagctggcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 57 - DNA, 45 bp, synthetic construct
  source 1..45: mol_type = other DNA
agggccagcaaaagtgtcagtacatctggctatagttatatgcac

SEQ ID NO: 58 - DNA, 32 bp, synthetic construct
  source 1..32: mol_type = other DNA
agggccagccaaagtattagcaacaacctaca

SEQ ID NO: 59 - DNA, 21 bp, synthetic construct
  source 1..21: mol_type = other DNA
cttgtatccaacctagaatct

SEQ ID NO: 60 - DNA, 21 bp, synthetic construct
  source 1..21: mol_type = other DNA
tatgcttcccagtccatctct

SEQ ID NO: 61 - DNA, 23 bp, synthetic construct
  source 1..23: mol_type = other DNA
cagcacattagggagcttacacg

SEQ ID NO: 62 - DNA, 27 bp, synthetic construct
  source 1..27: mol_type = other DNA
caacaaattaacagctggcctctcacg

SEQ ID NO: 63 - DNA, 69 bp, synthetic construct
  source 1..69: mol_type = other DNA
gacattgtgctgacacagtctcctgcttccttagctgtatctctggggcagagggccaccatctcatac

SEQ ID NO: 64 - DNA, 69 bp, synthetic construct
  source 1..69: mol_type = other DNA
gatattgtgctaactcagtctccagccaccctgtctgtgactccaggagatagcgtcagtctttcctgc

SEQ ID NO: 65 - DNA, 45 bp, synthetic construct
  source 1..45: mol_type = other DNA
tggaaccaacagaaaccaggacagccacccagactcctcatctat

SEQ ID NO: 66 - DNA, 46 bp, synthetic construct
  source 1..46: mol_type = other DNA
ctggtatcaacaaaaatcacatgagtctccaaggcttctcatcaag

SEQ ID NO: 67 - DNA, 96 bp, synthetic construct
  source 1..96: mol_type = other DNA
ggggtccctgccaggttcagtggcagtgggtctgggacagacttcaccctcaacatccatcctgtggaggaggaggatgctgcaacctattactgt

SEQ ID NO: 68 - DNA, 96 bp, synthetic construct
  source 1..96: mol_type = other DNA
gggatcccctccaggttcagtggcagtggatcagggacagatttcactctcagtatcaacagtgtggagactgaagattttggaatgtatttctgt

SEQ ID NO: 69 - DNA, 30 bp, synthetic construct
  source 1..30: mol_type = other DNA
ttcggaggggggaccaagctggaaataaaa

SEQ ID NO: 70 - DNA, 30 bp, synthetic construct
  source 1..30: mol_type = other DNA
ttcggtgctgggaccaagctggagctgaaa

SEQ ID NO: 71 - DNA, 37 bp, synthetic construct
  source 1..37: mol_type = other DNA
atgggtactgctgctctgggttccaggttccactggt

SEQ ID NO: 72 - DNA, 60 bp, synthetic construct
  source 1..60: mol_type = other DNA
atggttttcacacctcagatacttggacttatgcttttttggatttcagcctccagaggt

SEQ ID NO: 73 - protein, 7 aa, synthetic construct
YASQSIS

SEQ ID NO: 74 - protein, 43 aa, synthetic construct
MAQRAFPNPYADYNKSLAEGYFDAAGRLTPEFSQRLTNKIREC

SEQ ID NO: 75 - protein, 121 aa, synthetic construct
EVQLQQSGPVLVKPGASVKMSCKASGYTFTDYYMNWVKQSHGKSLEWIGVINPYNGHTNYNQKFKGKATLTVDKSSNTAYMELNSLTSEDSAVYYCARFPYYGSSYRVDYWGQGTTLTVSS

SEQ ID NO: 76 - protein, 107 aa, synthetic construct
DIVLTQSPATLSVTPGDSVSLSCRASQSISNNLHWYQQKSHESPRLLIKYASQSISGIPSRFSGSGSGTDFTLSINSVETEDFGMYFCQQINSWPLTFGAGTKLELK

SEQ ID NO: 77 - protein, 121 aa, synthetic construct
QVQLVQSGAEVKKPGASVKVSCKASGYTFTDYYMNWVRQAPGQGLEWIGVINPYNGHTNYNQKFKGRVTMTVDTSTSTVYMELSSLRSEDTAVYYCARFPYYGSSYRVDYWGQGTTVTVSS

SEQ ID NO: 78 - protein, 121 aa, synthetic construct
QVQLVQSGAEVKKPGASVKVSCKASGYTFTDYYMNWVRQAPGQGLEWIGVINPYNGHTNYNQKFKGRATMTVDTSTSTVYMELSSLRSEDTAVYYCARFPYYGSSYRVDYWGQGTTVTVSS

SEQ ID NO: 79 - protein, 121 aa, synthetic construct
QVQLVQSGAEVKKPGASVKVSCKASGYTFTDYYMNWVKQAPGQGLEWIGVINPYNGHTNYNQKFKGRATMTVDKSTSTVYMELSSLRSEDTAVYYCARFPYYGSSYRVDYWGQGTTVTVSS

SEQ ID NO: 80 - protein, 107 aa, synthetic construct
EIVMTQSPATLSVSPGERATLSCRASQSISNNLHWYQQKPGQAPRLLIKYASQSISGIPARFSGSGSGTEFTLTISSLQSEDFAVYYCQQINSWPLTFGGGTKLEIK

SEQ ID NO: 81 - protein, 107 aa, synthetic construct
EIVMTQSPATLSVSPGERATLSCRASQSISNNLHWYQQKPGQAPRLLIKYASQSISGIPSRFSGSGSGTEFTLTISSLQSEDFAVYYCQQINSWPLTFGGGTKLEIK

SEQ ID NO: 82 - protein, 107 aa, synthetic construct
EIVMTQSPATLSVSPGERATLSCRASQSISNNLHWYQQKPGQAPRLLIKYASQSISGIPSRFSGSGSGTEFTLTISSLQSEDFAVYFCQQINSWPLTFGGGTKLEIK